(19) United States
(12) Patent Application Publication
(10) Pub. No.: US 2023/0018670 A1
(43) Pub. Date: Jan. 19, 2023

(54) Bispecific GD2 and B7H3 Binding Molecules and Methods of Use

(71) Applicants: WIN THERAPEUTICS, INC., Madison, WI (US); INVENRA INC., Madison, WI (US); WISCONSIN ALUMNI RESEARCH FOUNDATION, Madison, WI (US)

(72) Inventors: Paul SONDEL, Madison, WI (US); Daniel Justin GERHARDT, Madison, WI (US)

(21) Appl. No.: 17/904,706

(22) PCT Filed: Feb. 20, 2021
(86) PCT No.: PCT/US2021/018939

§ 371 Date: Aug. 19, 2022

Related U.S. Application Data
(60) Provisional application No. 62/979,245, filed on Feb. 20, 2020.

(51) Int. Cl. C07K 16/30; C07K 16/28; A61K 47/68; A61P 35/00

(52) U.S. Cl. CPC C07K 16/3084; C07K 16/2827; A61K 47/6803; A61K 47/6879; A61K 47/6851; A61P 35/00; C07K 2317/31; C07K 2317/92; C07K 2317/732; A61K 2039/505

(57) ABSTRACT

Disclosed herein are multi-specific antibody constructs that simultaneously bind two tumor cell surface antigens, GD2 and B7H3. The monovalent arms targeting GD2 and B7H3 are each low to moderate affinity for their respective antigens. The multi-specific antibodies disclosed herein bind to target tumor cells when both arms of the antibody bind to their antigens, resulting in high specificity of the antibody for GD2 and B7H3 expressing tumor cells.

### 1. CROSS-REFERENCE TO RELATED APPLICATIONS

This application claims priority to and the benefit of U.S. Provisional Application No. 62/979,245, filed on Feb. 20, 2020, which application is hereby incorporated by reference in its entirety.

### 2. SEQUENCE LISTING

The instant application contains a Sequence Listing which has been submitted via EFS-Web and is hereby incorporated herein by reference in its entirety. Said ASCII copy, created on Month XX, 2021, is named XXXXXUS_sequencelisting.txt, and is X,XXX,XXX bytes in size

### 3. BACKGROUND

Diasialoganglioside (GD2) is expressed on many solid tumors as well as in the peripheral nervous system. Treatment with the anti-GD2 monoclonal antibody dinutuximab is currently standard of care for patients with neuroblastoma. However, dinutuximab causes extreme pain in treated patients, severely limiting its efficacy.

B7 Homolog 3 (B7H3), also known as Cluster of Differentiation 276 (CD276), is highly expressed on many solid tumors but is not expressed on nerve cells. B7H3 expression has been linked to poor prognosis in human patients and to invasive and metastatic potential of tumors in in vitro models.

There exists a need for cancer therapeutics with high specificity for tumor cells expressing GD2 and B7H3.

### 4. SUMMARY

The present disclosure provides various antibody constructs, pharmaceutical compositions, and methods of treatment.

In a first aspect, the disclosure herein provides a multi-specific antibody construct comprising a first antigen binding site (ABS) specific for a first tumor cell surface antigen, wherein the first tumor cell antigen is B7 Homolog 3 (B7H3) and further comprising a second ABS specific for a second tumor cell surface antigen, wherein the second tumor cell antigen is di sialoganglioside (GD2).

In some embodiments, the first ABS binds human B7H3 with an equilibrium dissociation constant (K_{D}) that is greater than 10 nM, the second ABS binds human GD2 with a K_{D} that is greater than 10 nM, and the antibody construct binds to a tumor cell expressing B7H3 and GD2 with a K_{D} that is less than 100 nM.

In some embodiments, the antibody construct exhibits lower binding to cells that express GD2 but not B7H3 compared to cells that express both GD2 and B7H3.

In preferred embodiments, the antibody construct binds less to nerve cells as compared to dinutuximab at comparable concentrations.

In some embodiments, the first ABS of the antibody construct comprises a first heavy chain variable region (VH) CDR1, a first VH CDR2, a first VH CDR3, a first light chain variable region (VL) CDR1, a first VL CDR2, and a first VL CDR3. In particular embodiments, the first ABS comprises a first VH CDR1 with the amino acid sequence of SEQ ID NO:22, a first VH CDR2 with the amino acid sequence of SEQ ID NO:27, a first VH CDR3 with the amino acid sequence of SEQ ID NO:32, a first VL CDR1 with the amino acid sequence of SEQ ID NO:7, a first VL CDR2 with the amino acid sequence of SEQ ID NO:12, and a first VL CDR3 with the amino acid sequence of SEQ ID NO:17. In preferred embodiments, the first ABS comprises a first heavy chain variable region (VH) with the amino acid sequence of SEQ ID NO: 2 and a first light chain variable region (VL) with the amino acid sequence of SEQ ID NO: 1.

In some embodiments, the second ABS of the antibody construct comprises a second heavy chain variable region (VH) CDR1, a second VH CDR2, a second VH CDR3, a second light chain variable region (VL) CDR1, a second VL CDR2, and a second VL CDR3. In particular embodiments, the second ABS comprises a second VH CDR1 with the amino acid sequence of SEQ ID NO:57, a second VH CDR2 with the amino acid sequence of SEQ ID NO:62, a second VH CDR3 with the amino acid sequence of SEQ ID NO:67, a second VL CDR1 with the amino acid sequence of SEQ ID NO:42, a second VL CDR2 with the amino acid sequence of SEQ ID NO:47, and a second VL CDR3 with the amino acid sequence of SEQ ID NO:52. In preferred embodiments, the second ABS comprises a second heavy chain variable region (VH) with the amino acid sequence of SEQ ID NO:4 and a second light chain variable region (VL) with the amino acid sequence of SEQ ID NO:3.

In some embodiments, the antibody construct comprises a first, second, third, and fourth polypeptide chain, wherein: the first polypeptide chain comprises a domain A, a domain B, a domain D, and a domain E, wherein the domains are arranged, from N-terminus to C-terminus, in a A-B-D-E orientation, wherein domain A comprises a variable region domain amino acid sequence, and wherein domain B, domain D, and domain E each comprise a constant region domain amino acid sequence; the second polypeptide chain comprises a domain F and a domain G, wherein the domains are arranged, from N-terminus to C-terminus, in a F-G orientation, and wherein domain F has a variable region domain amino acid sequence and domain G comprises a constant region domain amino acid sequence; the third polypeptide chain comprises a domain H, a domain I, a domain J, and a domain K, wherein the domains are arranged, from N-terminus to C-terminus, in a H-I-J-K orientation, wherein domain H has a variable region domain amino acid sequence, and wherein domain I, domain J, and domain K each have a constant region amino acid sequence; the fourth polypeptide chain comprises a domain L and a domain M, wherein the domains are arranged, from N-terminus to C-terminus, in a L-M orientation, and wherein domain L has a variable region domain amino acid sequence and domain M comprises a constant region amino acid sequence, or portion thereof; the first and second polypeptides are associated through an interaction between the A and F domains and an interaction between the B and G domains; the third and fourth polypeptides are associated through an interaction between the H and L domains and an interaction between the I and M domains; and the first and third polypeptides are associated through an interaction between the D and J domains and an interaction between the E and K domains. In some embodiments, domain A is a V_{L} domain; domain B comprises a CH3 domain; domain D is a CH2 domain; domain E is a CH3 domain; domain F is a V_{H} domain; domain G comprises a CH3 domain; domain H is a V_{L} domain; domain I is a C_{L} domain; domain J is a CH2 domain; domain K is a CH3 domain; domain L is a V_{H} domain; and domain M is a CH1 domain. In some embodiments, domains D and J have the amino acid sequence of human IgG1 CH2 domain; domain I has the amino acid sequence of human C kappa light chain; and domain M has the amino acid sequence of human IgG1 CH1 region.

In some embodiments, domain B has a CH3 amino acid sequence with a T366K mutation and a C-terminal extension incorporating a KSC tripeptide sequence followed by the DKTHT motif of an IgG1 hinge region; domain E has a CH3 amino acid sequence with a S354C and a T366W mutation; domain G has a CH3 amino acid sequence with a L351D mutation and a C-terminal extension incorporating a GEC amino acid disulfide motif; and domain K has a CH3 amino acid sequence with a Y349C, a T366S, a L368A, a Y407V mutation, and optionally a D356E and a L358M mutation

In some embodiments, domain B has a CH3 amino acid sequence and a C-terminal extension incorporating a KSC tripeptide sequence followed by the DKTHT motif of an IgG1 hinge region; domain E has a CH3 amino acid sequence with a S354C and a T366W mutation; domain G has a CH3 amino acid sequence and a C-terminal extension incorporating a GEC amino acid disulfide motif; and domain K has a CH3 amino acid sequence with a Y349C, a T366S, a L368A, a Y407V mutation, and optionally a D356E and a L358M mutation.

In some embodiments, domain B has a CH3 amino acid sequence with a Y349C mutation and a C-terminal extension incorporating a PGK tripeptide sequence that is followed by the DKTHT motif of an IgG1 hinge region; domain E has a CH3 amino acid sequence with a S354C and a T366W mutation; domain G has a CH3 amino acid sequence with a S354C mutation and a C-terminal extension incorporating a PGK tripeptide sequence; and domain K has a CH3 amino acid sequence with a Y349C, a T366S, a L368A, a Y407V mutation, and optionally a D356E and a L358M mutation.

In some embodiments, domain B has a CH3 amino acid sequence with a P343 V mutation, a Y349C mutation, and a C-terminal extension incorporating a PGK tripeptide sequence that is followed by the DKTHT motif of an IgG1 hinge region; domain E has a CH3 amino acid sequence with a S354C and a T366W mutation; domain G has a CH3 amino acid sequence with a S354C mutation and a C-terminal extension incorporating a PGK tripeptide sequence; and domain K has a CH3 amino acid sequence with a Y349C, a T366S, a L368A, a Y407V mutation, and optionally a D356E and a L358M mutation.

In some embodiments, the first ABS is formed by domains A and F and the second ABS is formed by domains H and L.

In some embodiments, the antibody construct is conjugated to a therapeutic agent.

In another aspect, disclosed herein is a pharmaceutical composition comprising an effective amount of a multi-specific antibody construct described herein and a pharmaceutically acceptable carrier.

In another aspect, disclosed herein is a method of treating a proliferative disease in a human subject, comprising administering to the human subject a pharmaceutical composition comprising an effective amount of an antibody construct described herein. In some embodiments, the proliferative disease is cancer. In some embodiments, the cancer is neuroblastoma, glioblastoma, small cell lung cancer, or sarcoma.

In some embodiments, administering the pharmaceutical composition results in decreased pain compared to treatment with an anti-GD2 monoclonal antibody. In some embodiments, the anti-GD2 monoclonal antibody is dinutuximab or hu14.18

In another aspect, disclosed herein is a method of selectively targeting a tumor cell in a subject, comprising administering to the subject a pharmaceutical composition comprising an effective amount of an antibody construct described herein.

### 5. BRIEF DESCRIPTION OF THE DRAWINGS

These and other features, aspects, and advantages of the present invention will become better understood with regard to the following description, and accompanying drawings, where:

FIG. 1 is a slide outlining the need for the antibody constructs disclosed herein and the approach to solving that need that is met by the antibody constructs disclosed herein.

FIG. 2 identifies the goal of creating the antibody constructs disclosed herein.

FIG. 3A, FIG. 3B, FIG. 3C, and FIG. 3D are schematics comparing anti-GD2 antibody which binds to GD2 antigen (FIG. 3A) on neuroblastoma and peripheral nerve cells (FIG. 3B) with anti-GD2×B7H3 SNIPER™ antibody (INV721) that binds B7H3 antigen (FIG. 3C) on neuroblastoma cells but not nerve cells (FIG. 3D).

FIG. 4 outlines the project workflow for generating the antibody constructs disclosed herein.

FIG. 5 identifies desired antibody-dependent cellular cytotoxicity (ADCC) criteria for the antibody constructs disclosed herein.

FIG. 6 provides the amino acid sequences of two GD2 antigen binding molecules.

FIG. 7 outlines the protocol for a GD2 binding assay to assess binding of potential GD2 binding molecules to GD2 expressing tumor cells.

FIG. 8 provides results of an assay evaluating binding of four anti-GD2 antibodies to M21 melanoma cells that express GD2 and B7H3.

FIG. 9 provides results of an assay evaluating binding of four anti-GD2 antibodies to M21 and B78 melanoma cells that express GD2 and B7H3.

FIG. 10 identifies the number of B7H3 binding molecules evaluated and the number of B7H3 binding molecules that remained candidates for the antibody constructs disclosed herein.

FIG. 11 outlines the protocol for a B7H3 binding assay to assess binding of potential B7H3 binding molecules to B7H3 expressing tumor cells.

FIG. 12 provides results of assays evaluating binding of four anti-B7H3 antibodies to M21 melanoma cells and B16 cells that express GD2 and B7H3.

FIG. 13 provides results of assays evaluating binding of four anti-B7H3 antibodies and three anti-GD2 antibodies to B16 and B78 cells.

FIG. 14 summarizes the GD2 and B7H3 binding molecules evaluated.

FIG. 15 provides results of assays evaluating binding of six GD2×B7H3 bispecific antibodies and the anti-GD2 monoclonal antibody dinutuximab to M21 melanoma cells.

FIG. 16 provides results of assays evaluating binding of four GD2×B7H3 bispecific antibodies to B16 cells.

FIG. 17 summarizes initial results of binding studies of GD2×B7H3 bispecific antibody candidates.

FIG. 18 provides the amino acid sequences of two GD2 antigen binding molecules.

FIG. 19 provides the results of epitope binning of various candidate GD2×B7H3 bispecific antibodies.

FIG. 20 summarizes features and characteristics of candidate GD2×B7H3 bispecific antibodies.

FIG. 21 outlines studies for assessing binding of various candidate GD2×B7H3 bispecific antibodies.

FIG. 22 identifies chromatography assays for use in assessing candidate GD2×B7H3 bispecific antibodies.

FIG. 23 shows results of binding and chromatography assays of GD2×B7H3 bispecific antibody candidate I7-01×GD2-5.

FIG. 24 shows results of binding and chromatography assays of GD2×B7H3 bispecific antibody candidate I7-01×GD2-6.

FIG. 25 shows results of binding and chromatography assays of GD2×B7H3 bispecific antibody candidate I7-09×GD2-5.

FIG. 26 shows results of binding and chromatography assays of GD2×B7H3 bispecific antibody candidate I7-09×GD2-6.

FIG. 27 shows results of binding and chromatography assays of GD2×B7H3 bispecific antibody candidate I7-10×GD2-5.

FIG. 28 shows results of binding and chromatography assays of GD2×B7H3 bispecific antibody candidate I7-10×GD2-6.

FIG. 29 shows results of binding and chromatography assays of GD2×B7H3 bispecific antibody candidate I7-19×GD2-5.

FIG. 30 shows results of binding and chromatography assays of GD2×B7H3 bispecific antibody candidate I7-19×GD2-6.

FIG. 31 shows results of binding and chromatography assays of GD2×B7H3 bispecific antibody candidate I7-20×GD2-6.

FIG. 32 shows results of binding and chromatography assays of GD2×B7H3 bispecific antibody candidate I7-33×GD2-5.

FIG. 33 shows results of binding and chromatography assays of GD2×B7H3 bispecific antibody candidate I7-33×GD2-6.

FIG. 34 shows results of binding and chromatography assays of GD2×B7H3 bispecific antibody candidate I7-34×GD2-5.

FIG. 35 shows results of binding and chromatography assays of GD2×B7H3 bispecific antibody candidate I7-34×GD2-6.

FIG. 36 shows results of binding and chromatography assays of GD2×B7H3 bispecific antibody candidate I7-36×GD2-5.

FIG. 37 shows results of binding and chromatography assays of GD2×B7H3 bispecific antibody candidate I7-36×GD2-6.

FIG. 38 identifies candidate GD2×B7H3 bispecific antibodies for testing by antibody-directed cellular cytotoxicity (ADCC) assay.

FIG. 39 summarizes the evaluation of candidate GD2×B7H3 bispecific antibodies.

FIG. 40 outlines the protocol for antibody-directed cellular cytotoxicity (ADCC) assays to assess efficacy of candidate GD2×B7H3 bispecific antibodies.

FIG. 41 shows ADCC assay results of various candidate GD2×B7H3 bispecific antibodies.

FIG. 42 shows results of repeated ADCC assays of various candidate GD2×B7H3 bispecific antibodies.

FIG. 43 is a schematic overview of the protocol for a model system for evaluating pain.

FIG. 44 shows results of a pilot study evaluating pain following treatment with the anti-GD2 monoclonal antibody dinutuximab.

FIG. 45 summarizes the results of a pilot study evaluating pain following dinutuximab treatment and identifies candidate GD2×B7H3 bispecific antibodies for further study.

FIG. 46 identifies potential therapeutic indications for GD2×B7H3 SNIPER™ antibodies.

FIG. 47 identifies uses potential uses of modified GD2×B7H3 SNIPER™ antibodies.

FIG. 48 summarizes studies performed to evaluate candidate GD2×B7H3 bispecific antibodies.

FIG. 49 shows results of binding assays comparing various GD2×B7H3 bispecific antibody candidates and dinutuximab in B78 cells that express GD2 and B7H3 (A), GD2 only (B), or B7H3 only (C). Positron emission topography (PET) images of mice bearing GD2 and B7H3 expressing B78 tumors are shown in (D) following treatment with a radiolabeled GD2×B7H3 antibody candidate (I7-33×GD2-2) (top panel) or a radiolabeled non-specific control antibody (BBody). Biodistribution of radiolabeled I7-33×GD2-2 was assessed and compared to biodistribution of radiolabeled dinutuximab; results shown in (E).

FIG. 50 outlines experiments performed to evaluate the anti-GD2, anti-B7H3 (GD2×B7H3) bispecific SNIPER™ antibody INV721.

FIG. 51 presents a schematic architecture, with respective naming conventions, for various bivalent antibody constructs described herein.

FIG. 52A and FIG. 52B show results of binding assays of INV721 in B78 cells expressing the tumor antigens GD2 and B7H3 (FIG. 52A) and in B78 cells expressing GD2 but not B7H3 (FIG. 52B).

FIG. 53 is a table showing relative expression of the tumor cell surface antigens GD2 and B7H3 in various pediatric cancer cell lines.

FIG. 54 is a table showing relative expression of the tumor cell surface antigens GD2 and B7H3 in various melanoma cell lines.

FIG. 55A, FIG. 55B, and FIG. 55C show results of studies evaluating internalization of INV721 and anti-GD2 and anti-B7H3 monoclonal antibodies in various pediatric neuroblastoma cell lines: CHLA20 (FIG. 55A), LAN-1 (FIG. 55B), and NGP (FIG. 55C). Antibody internalization results in an increase in total red object integrated intensity.

FIG. 56A and 56B show results of antibody-directed cellular cytotoxicity (ADCC) assays of B78 tumor spheroids treated with INV721 (FIG. 56A) or separately treated with INV721 or the anti-GD2 antibody Hu14.18 (FIG. 56B). Dispersion of the bright compact signal following treatment with INV721 demonstrates that INV721 inhibits tumor growth via ADCC.

FIG. 57A, FIG. 57B, and FIG. 57C show results of direct antibody-mediated apoptosis assays evaluating B78 tumor cells treated with INV721 and peripheral blood mononuclear cells (PBMCs), INV721 alone, or PBMCs alone. Experiments were performed in B78 tumor cells expressing GD2 and B7H3 (FIG. 57A), expressing GD2 but not B7H3 (FIG. 57B), and expressing B7H3 but not GD2 (FIG. 57C).

FIG. 58A, FIG. 58B, and FIG. 58C show results of ADCC assays comparing INV721 and the anti-GD2 antibodies dinutuximab and Hu14.18 in various melanoma cell lines that express GD2 and B7H3: M21 (FIG. 58A), Mel7 (FIG. 58B), and Mel13 (FIG. 58C).

FIG. 59 shows results of ADCC assays comparing cytotoxicity effects of INV-721, anti-GD2 antibodies dinutuximab and GD2-7, and anti-B7H3 antibody I7-01 in M21 cells that express GD2 and B7H3.

FIG. 60 is a cartoon diagram illustrating the localization and GD2/B7H3 expression status of tumors in mice evaluated for binding of radiolabeled INV721 and dinutuximab.

FIG. 61 shows positron emission tomography (PET) images of mice bearing four tumors with differential GD2 and B7H3 expression and treated with radiolabeled antibody: INV721 (top panel), non-specific control (middle panel), and dinutuximab (bottom panel).

FIG. 62A, FIG. 62B, FIG. 62C, and FIG. 62D are graphs showing the ratio of activity of radiolabeled antibody injected per dose per gram of tumor in tumors with differential GD2 and B7H3 expression: GD2−/B7H3+ (FIG. 62A), GD2−/B7H3− (FIG. 62B), GD2+/B7H3− (FIG. 62C), and GD2+/B7H3+ (FIG. 62D).

FIG. 63A shows an exemplary image of a mouse evaluated for binding of radiolabeled antibody along its spine. FIG. 63B shows results of radiolabeled antibody binding of INV721 and dinutuximab, demonstrating that INV721 binds to the spine of treated mice significantly less than dinutuximab.

FIG. 64 is a schematic overview of the treatment protocol used for evaluating efficacy of INV721 in B78 melanoma tumors in mice.

FIG. 65 shows results of tumor growth and inhibition in mice bearing B78 melanoma tumors following no treatment (panel 1, from left), treatment with radiation therapy only (panel 2), treatment with radiation therapy and IL-2 (panel 3), treatment with radiation therapy and INV721 (panel 4), and treatment with radiation therapy, INV721, and IL-2 (panel 5).

FIG. 66 shows results of tumor growth and inhibition in mice bearing B78 melanoma tumors following no treatment, treatment with radiation therapy only, treatment with radiation therapy and IL-2, treatment with radiation therapy and INV721 (“I7-01/GD2-7”), treatment with radiation therapy and a-fucosylated INV721 (“A-Fuco I7-01/GD2-7”), treatment with radiation therapy and dinutuximab (“Din”), treatment with radiation therapy, INV721, and IL-2, treatment with radiation therapy, a-fucosylated INV721, and IL-2, and treatment with radiation therapy, dinutuximab, and IL-2.

FIG. 67 shows overall survival of mice bearing B78 melanoma tumors following no treatment, treatment with radiation therapy only, treatment with radiation therapy and IL-2, treatment with radiation therapy and INV721, and treatment with radiation therapy, INV721, and IL-2.

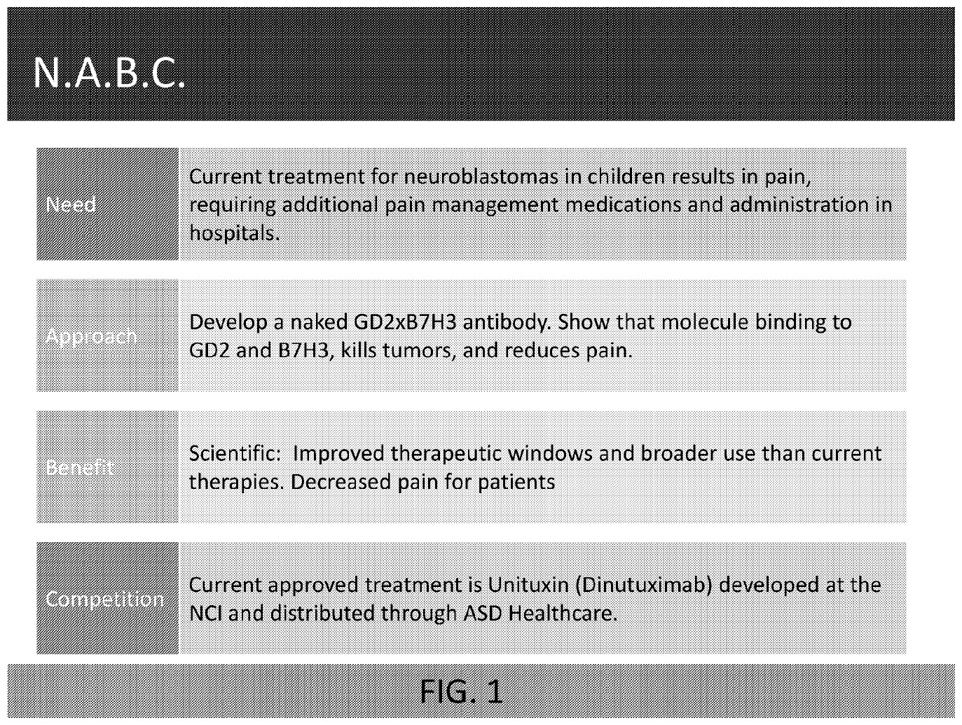

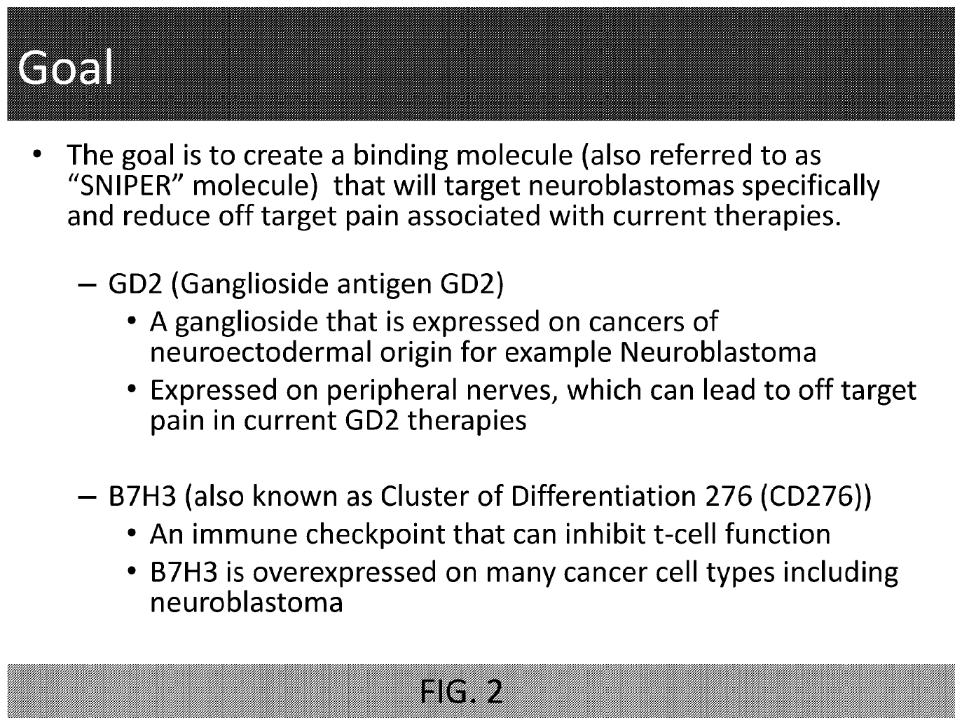

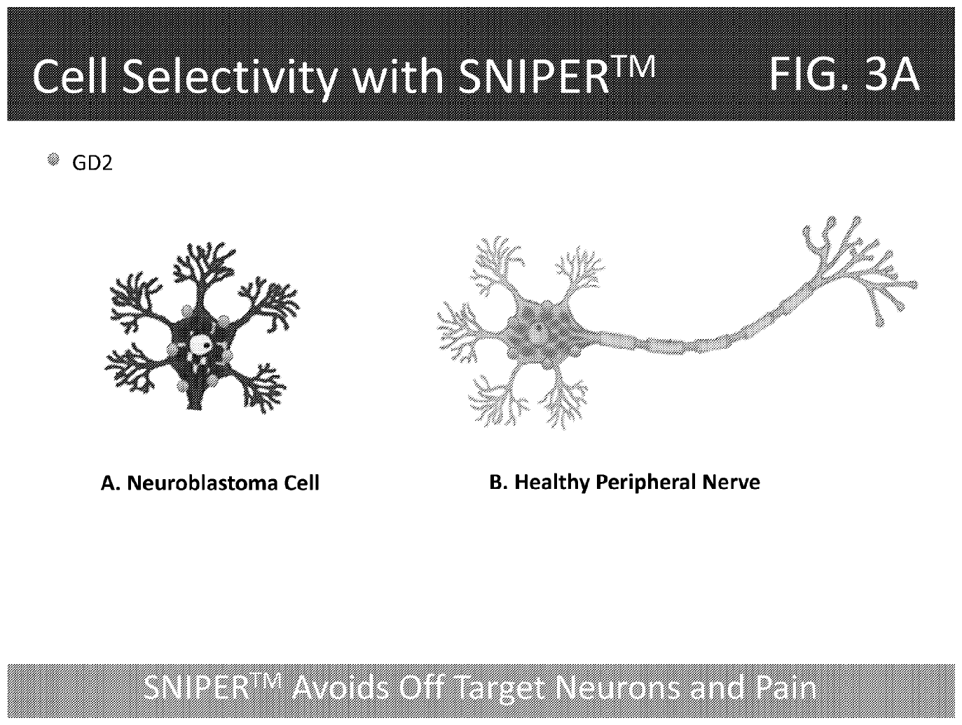

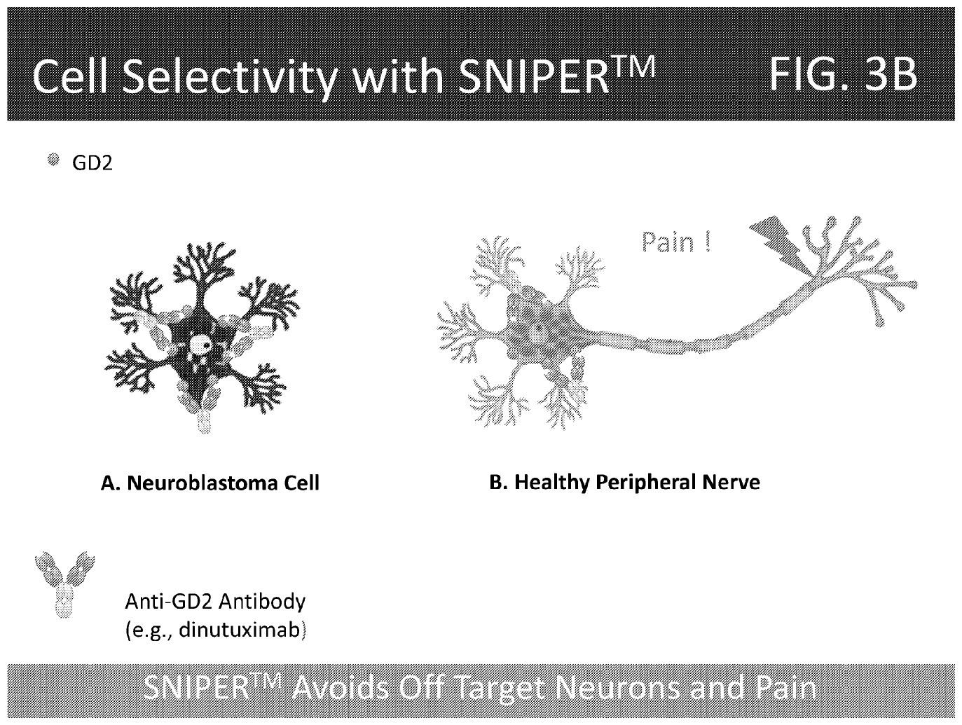

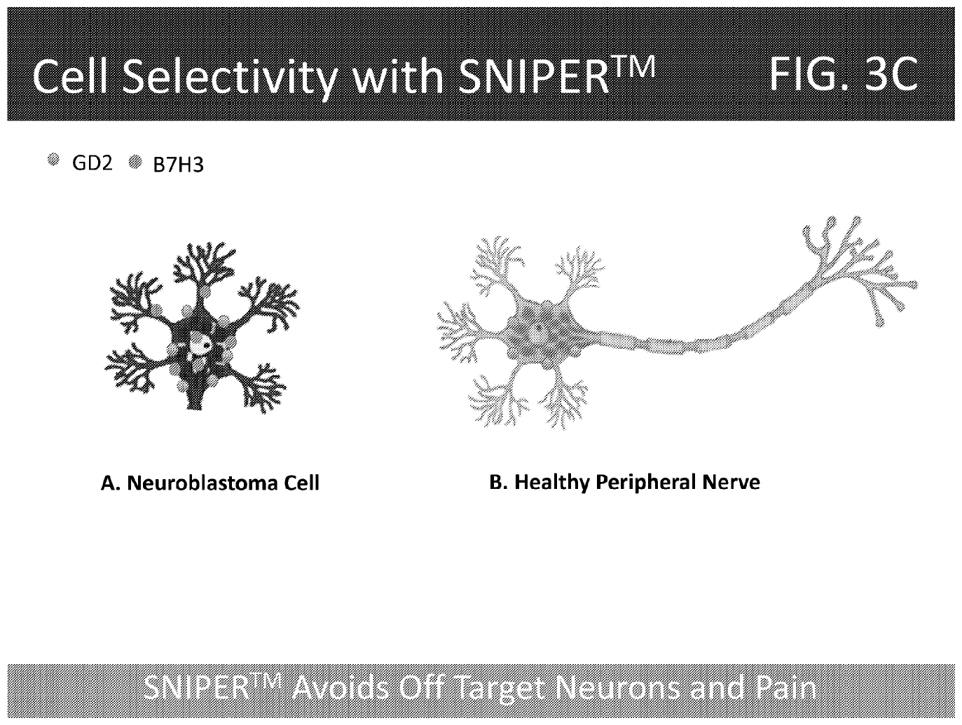

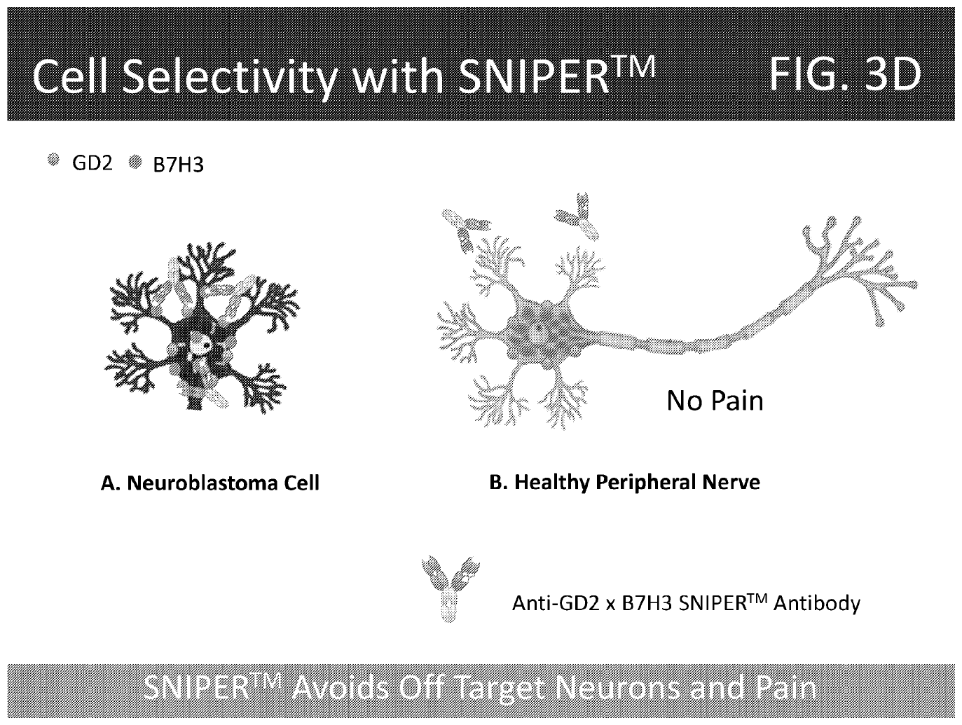

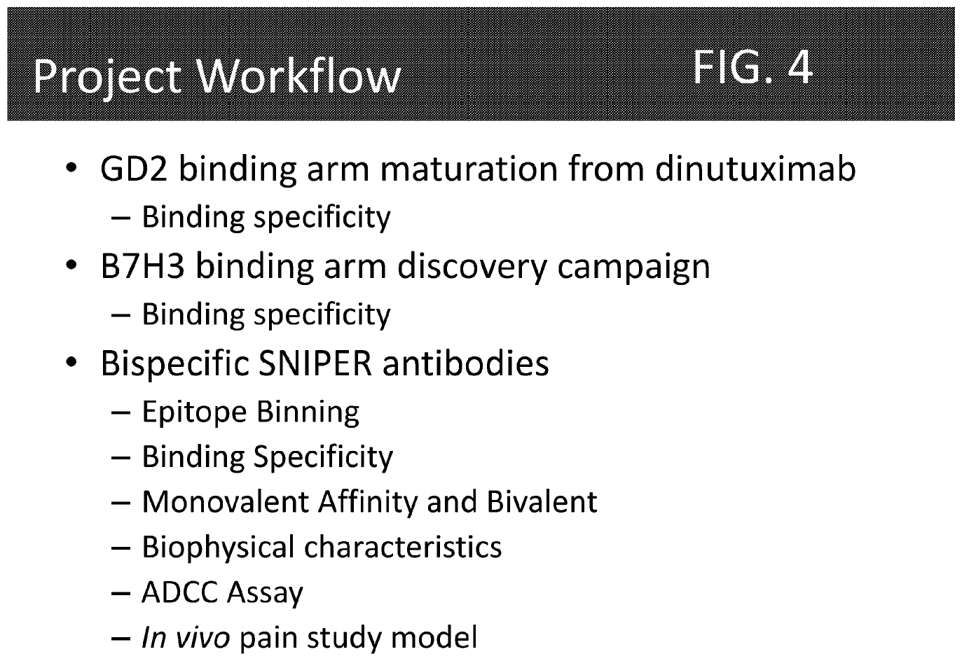

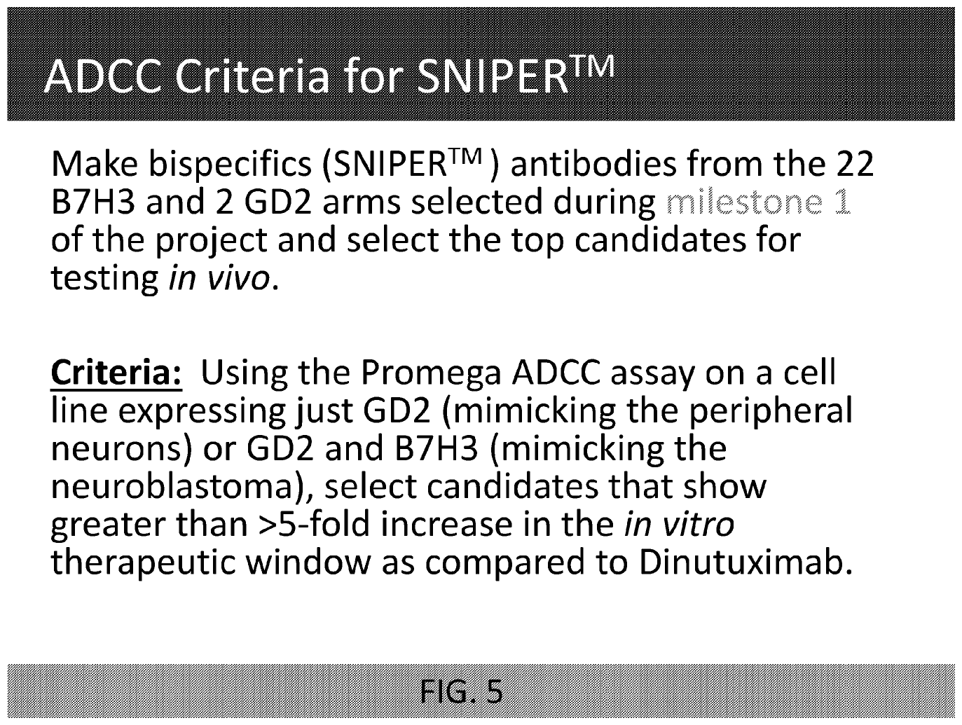

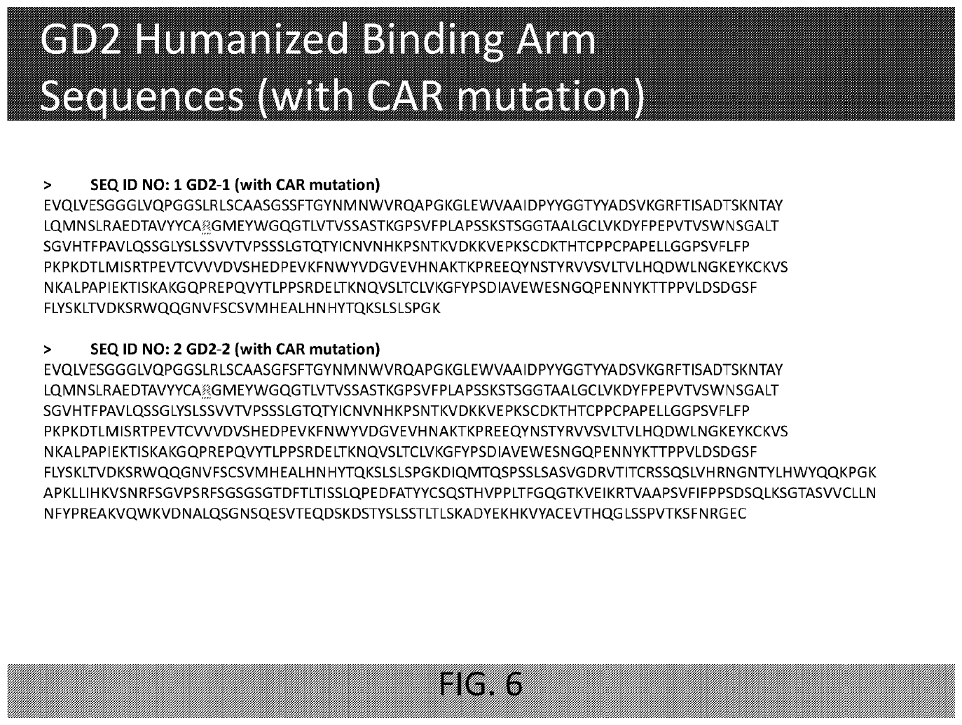

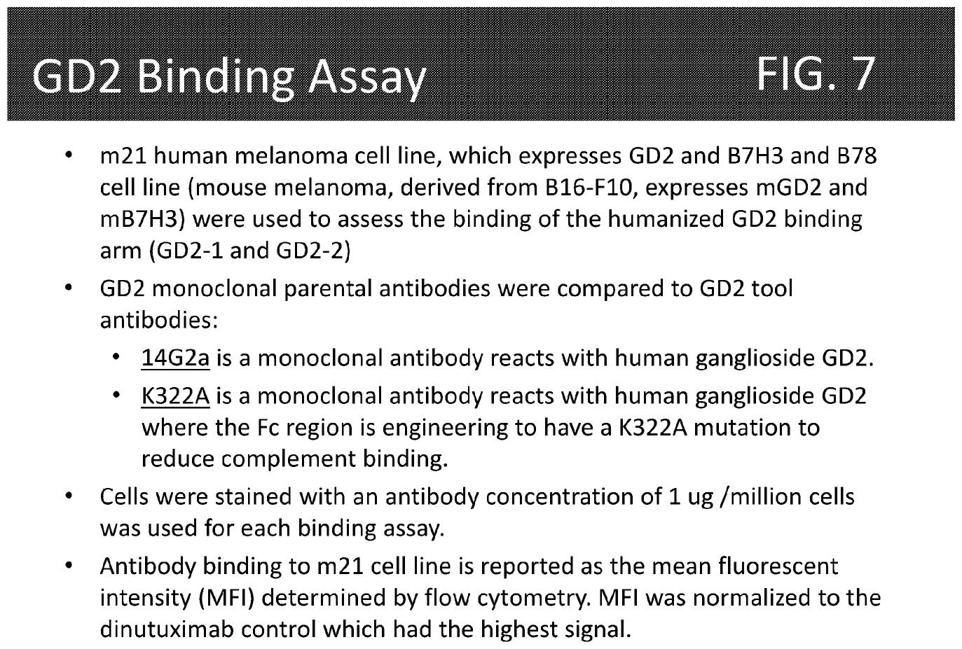

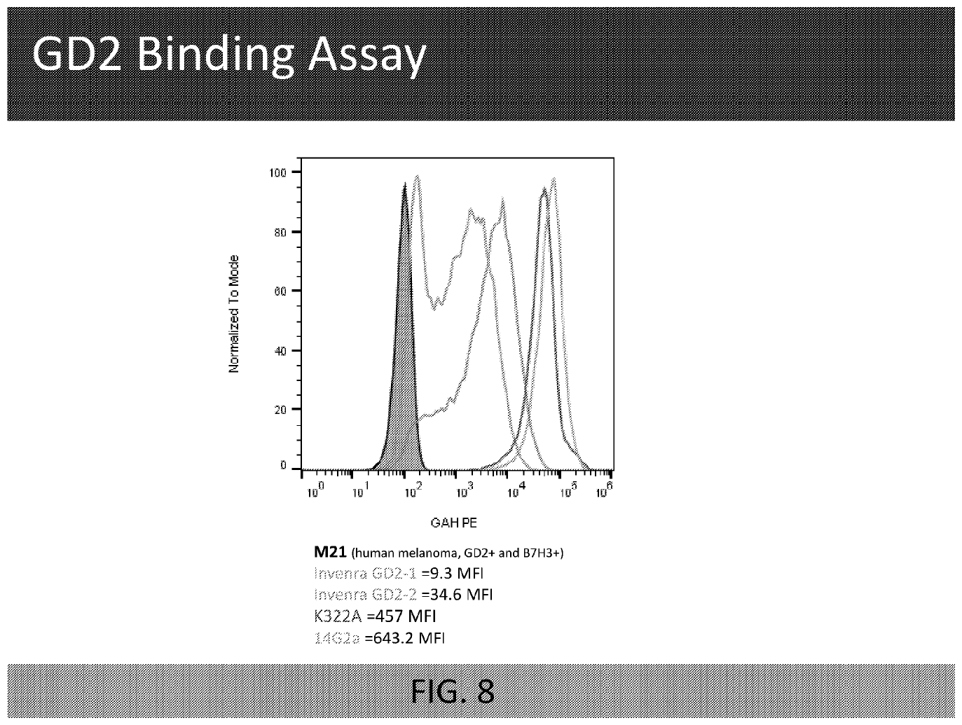

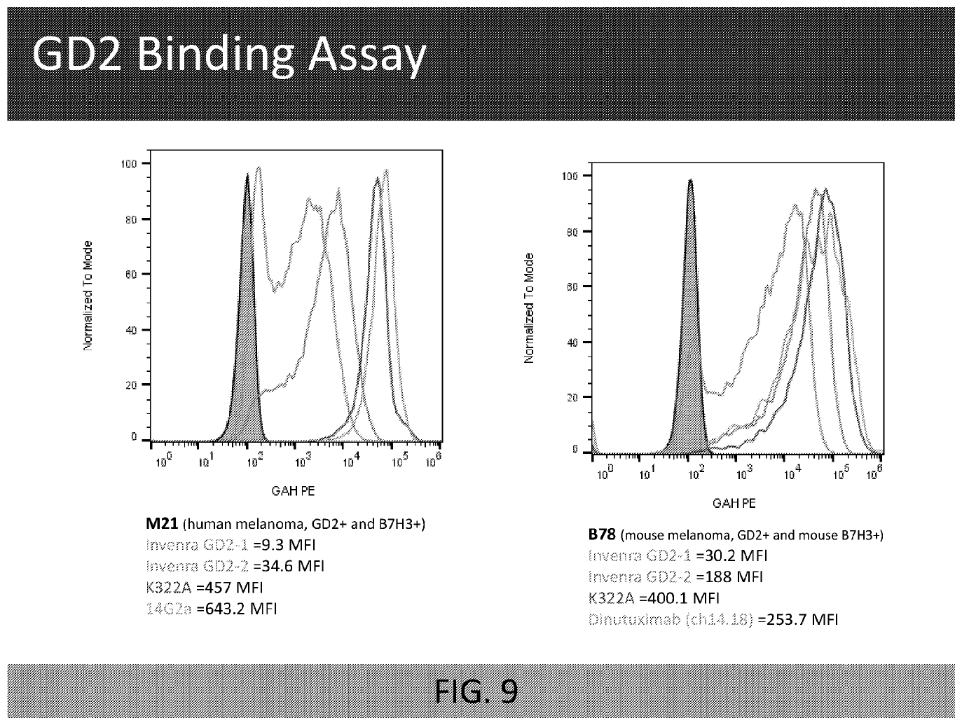

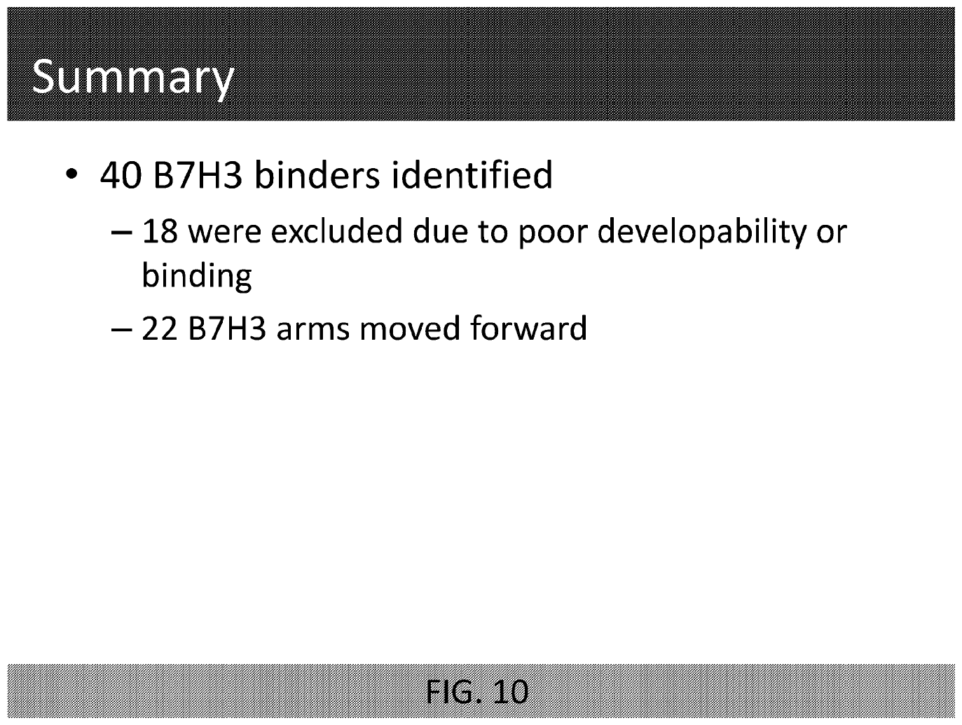

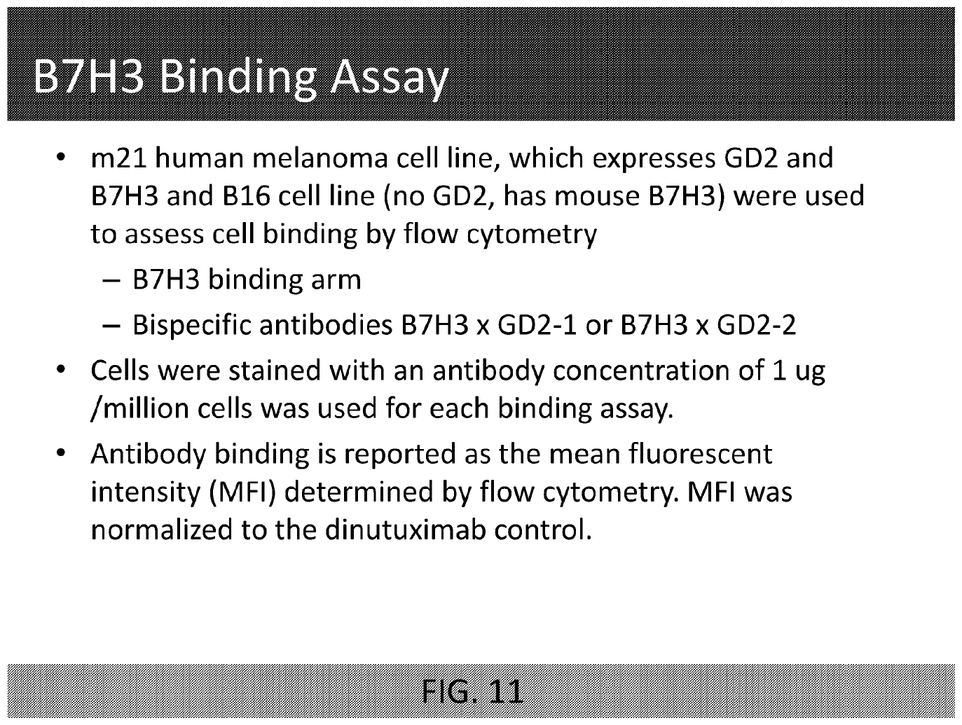

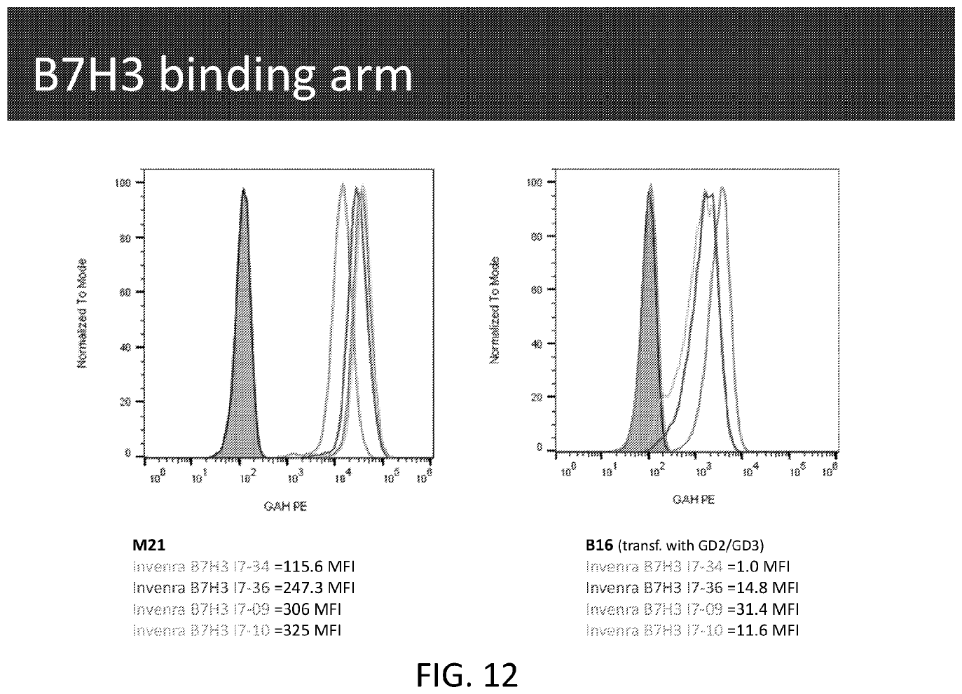

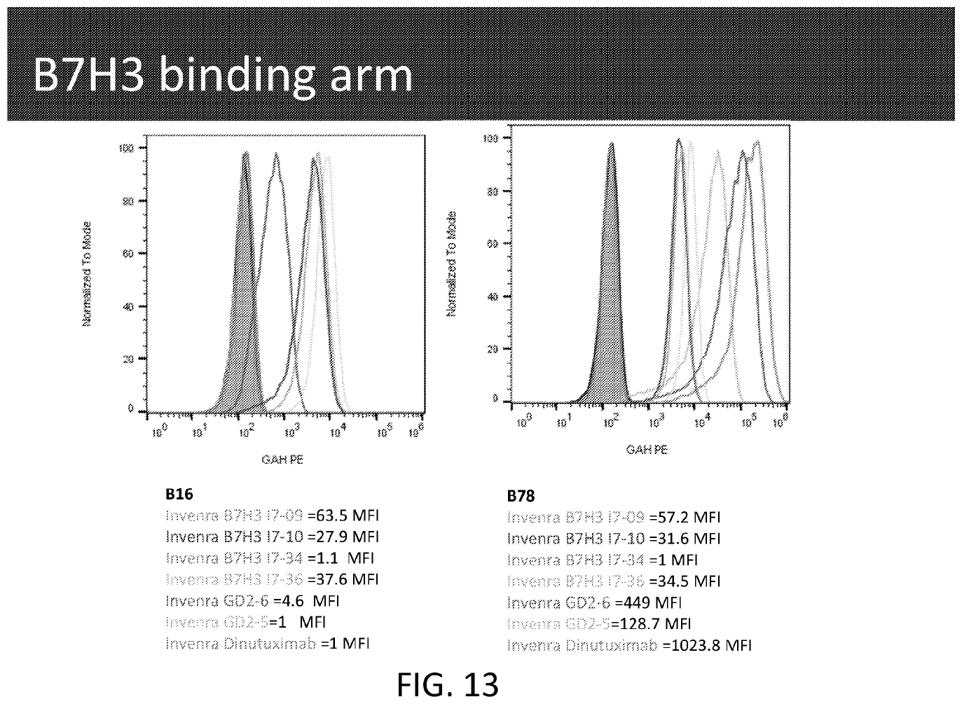

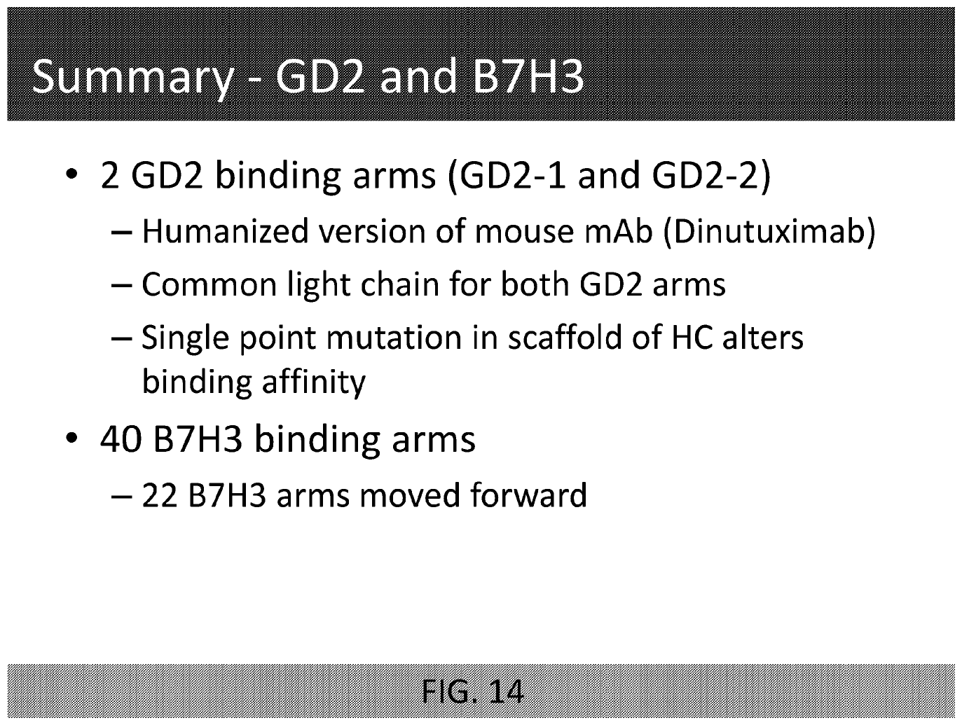

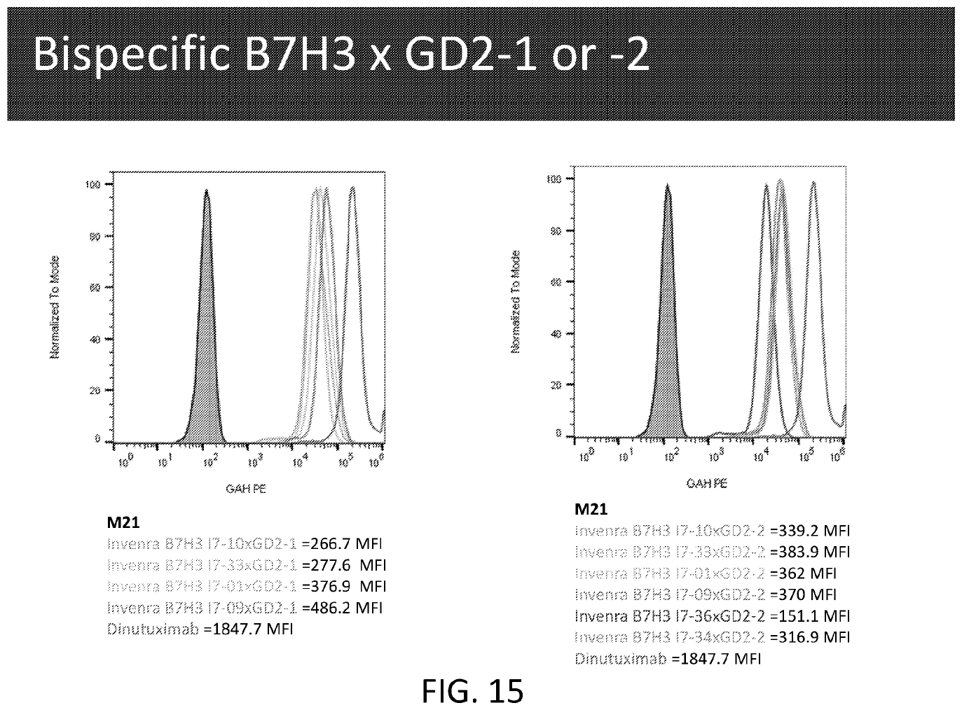

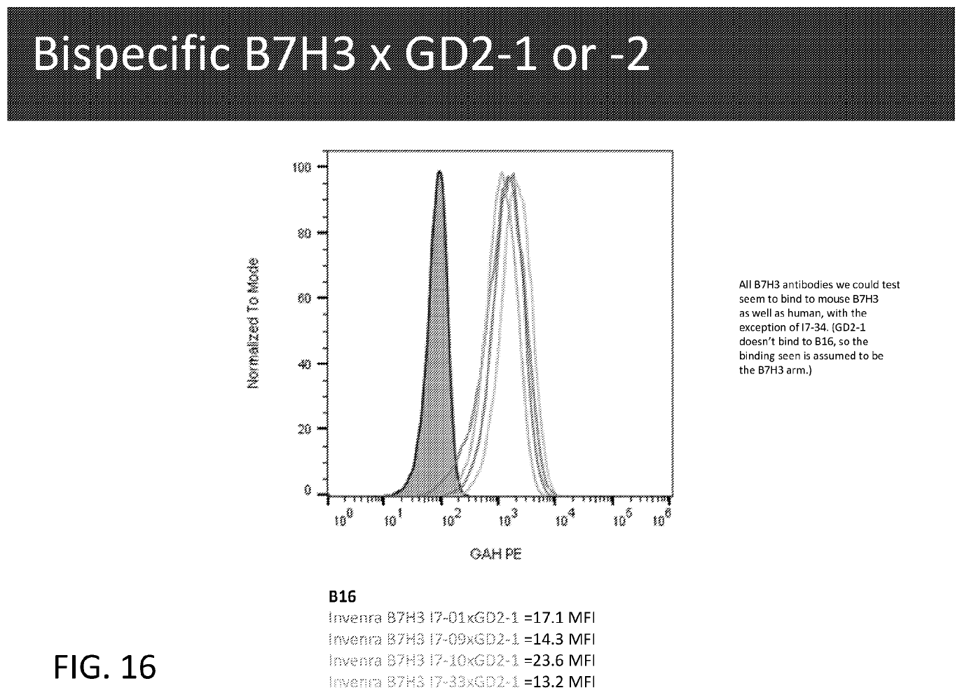

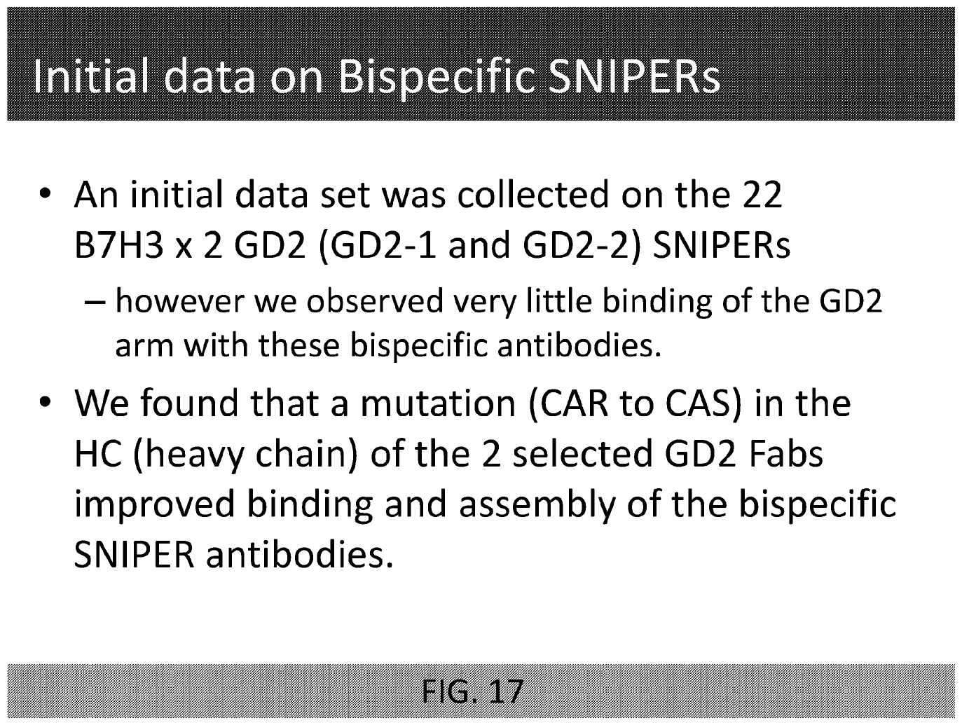

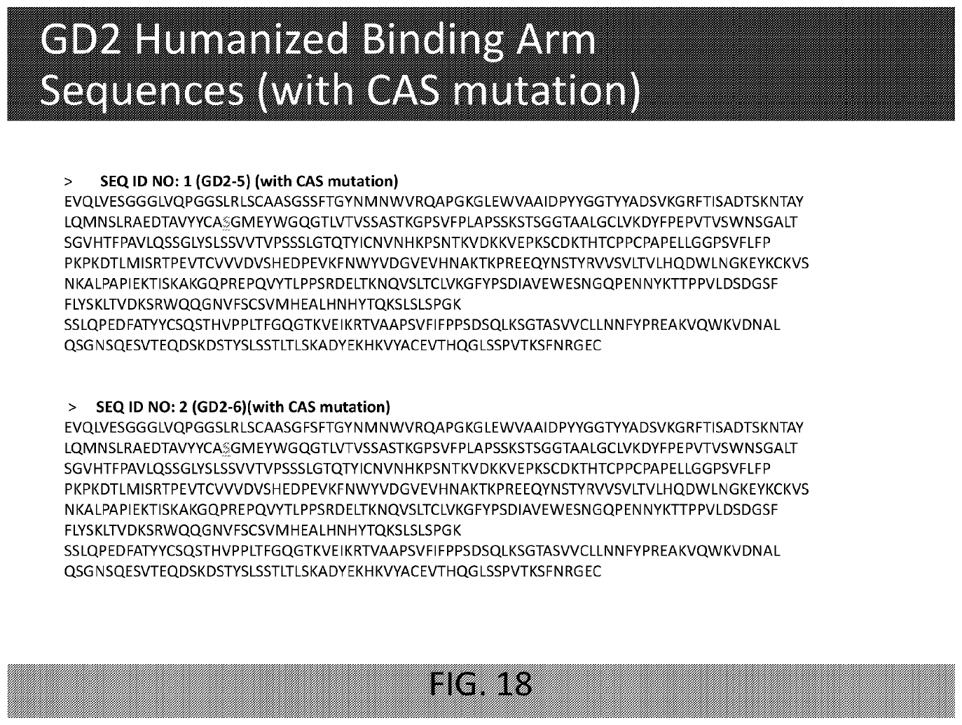

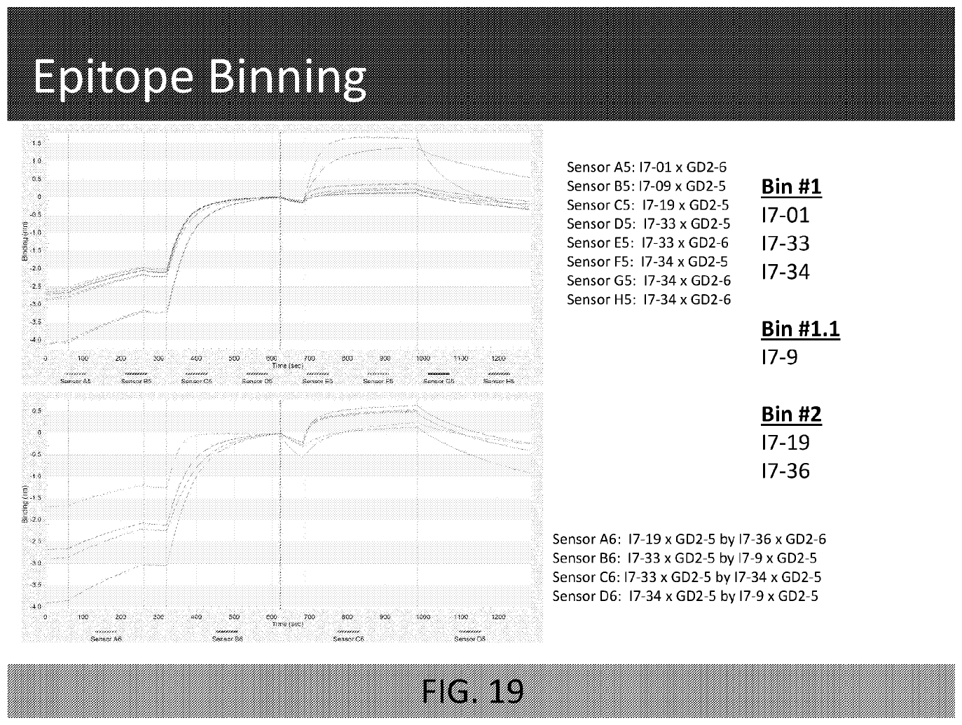

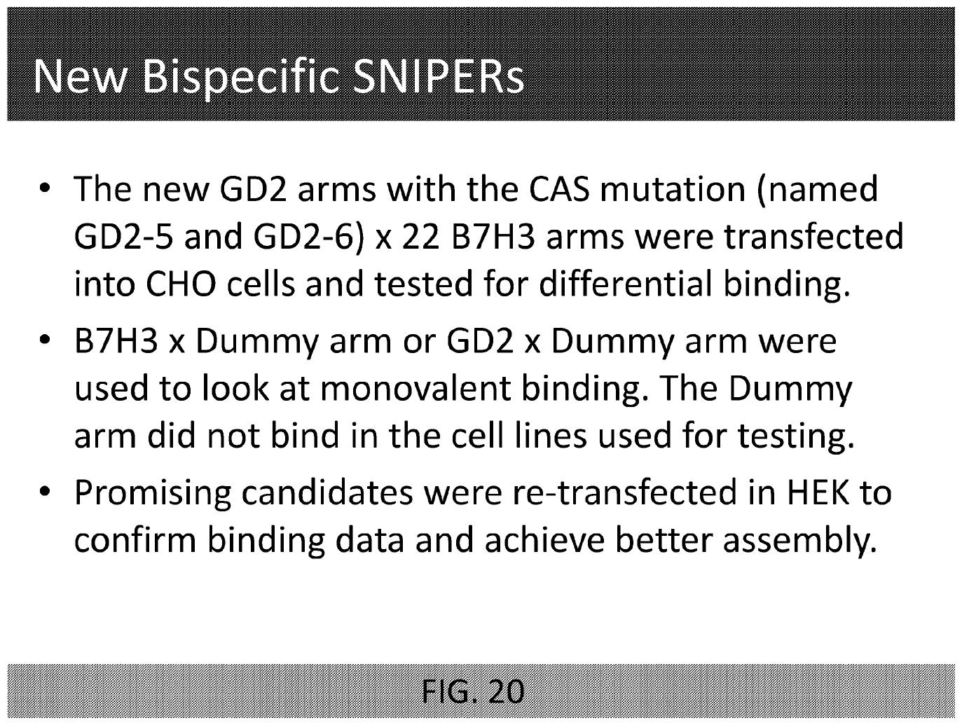

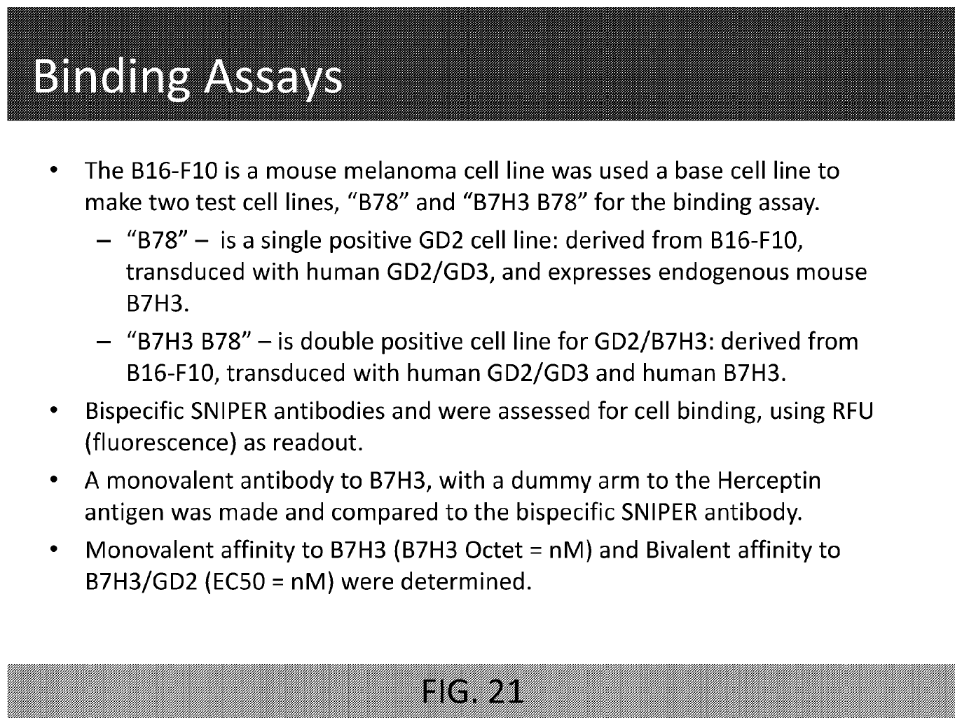

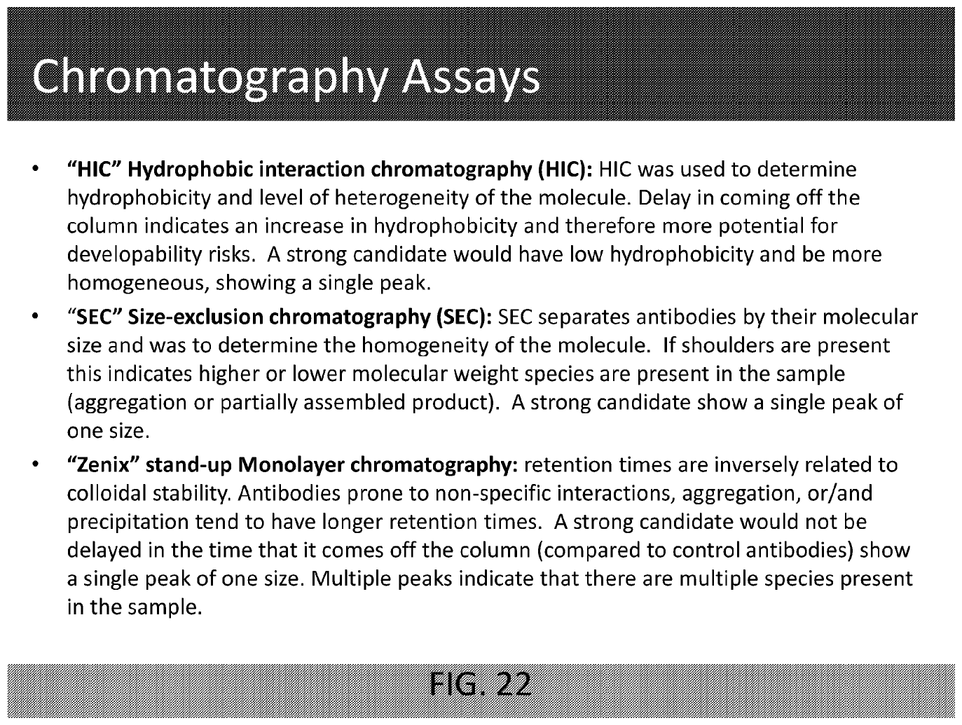

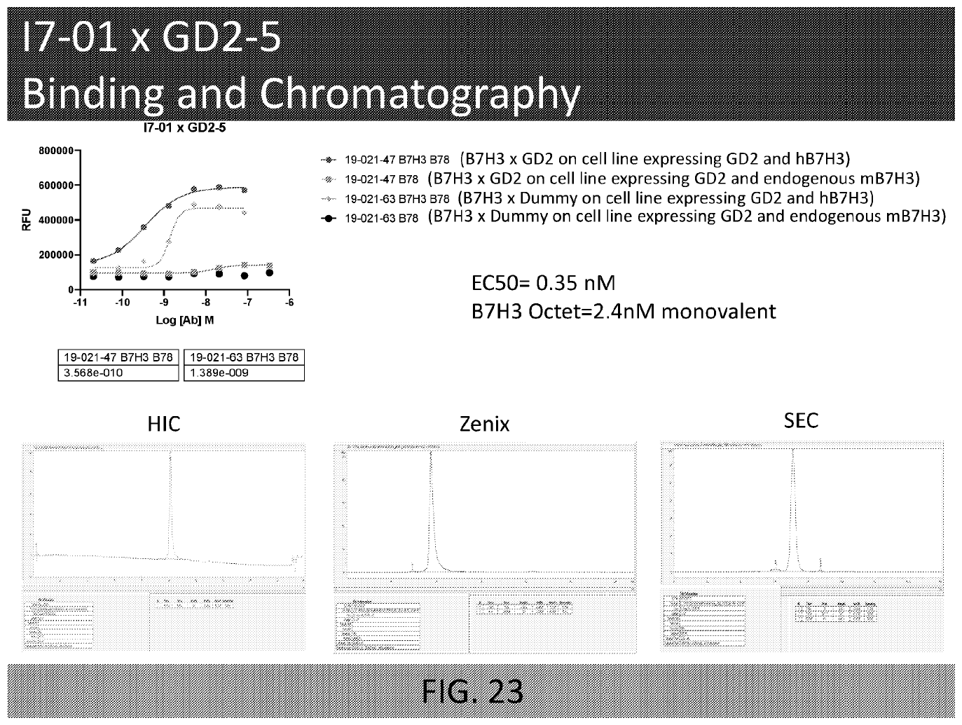

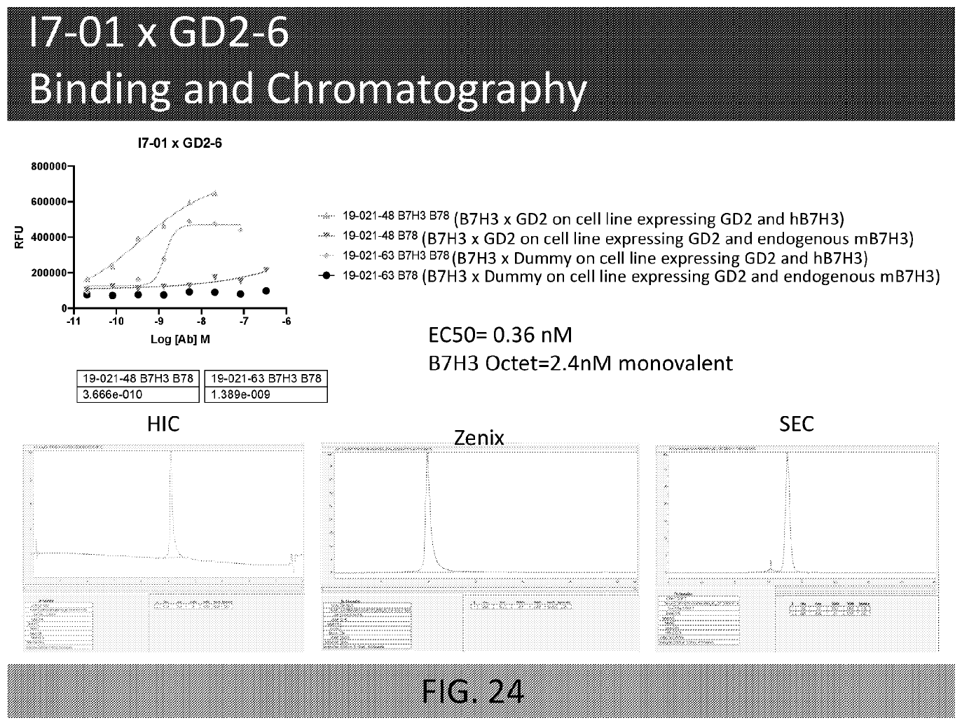

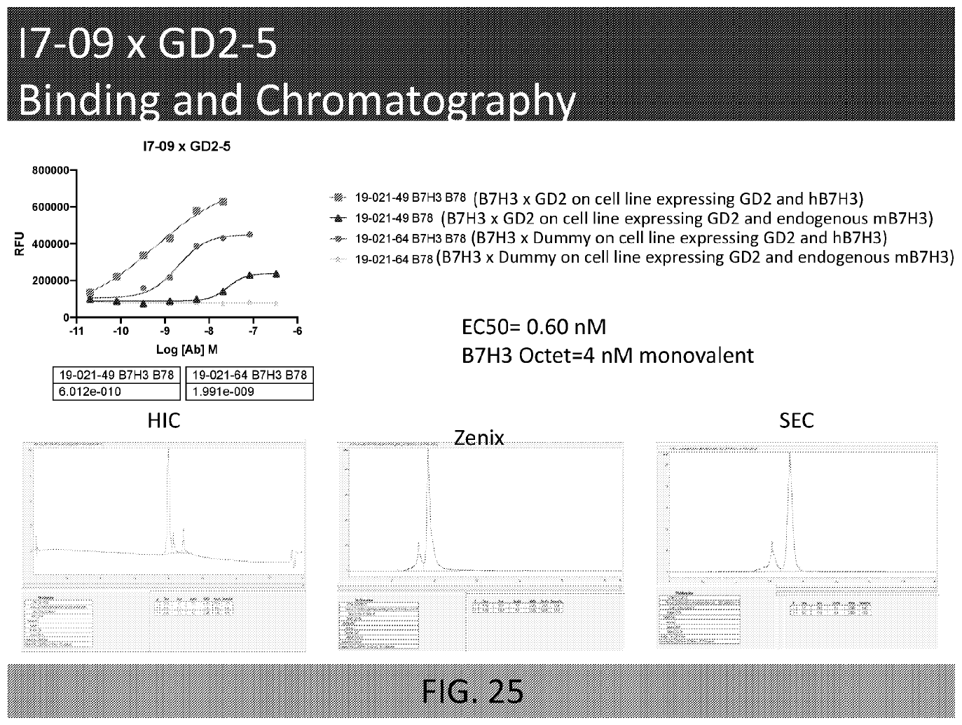

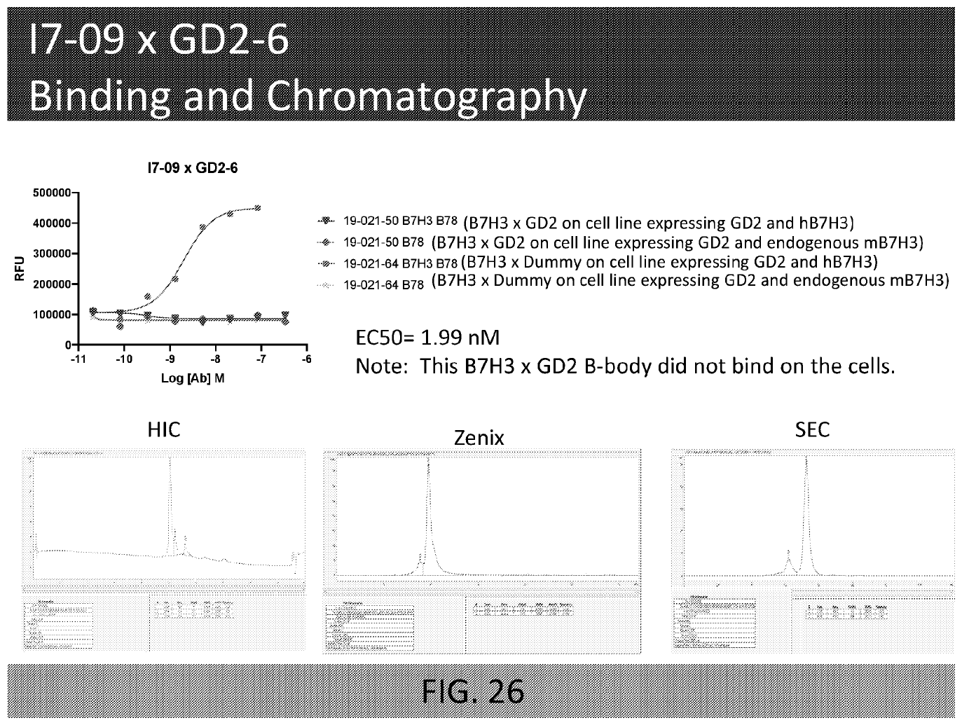

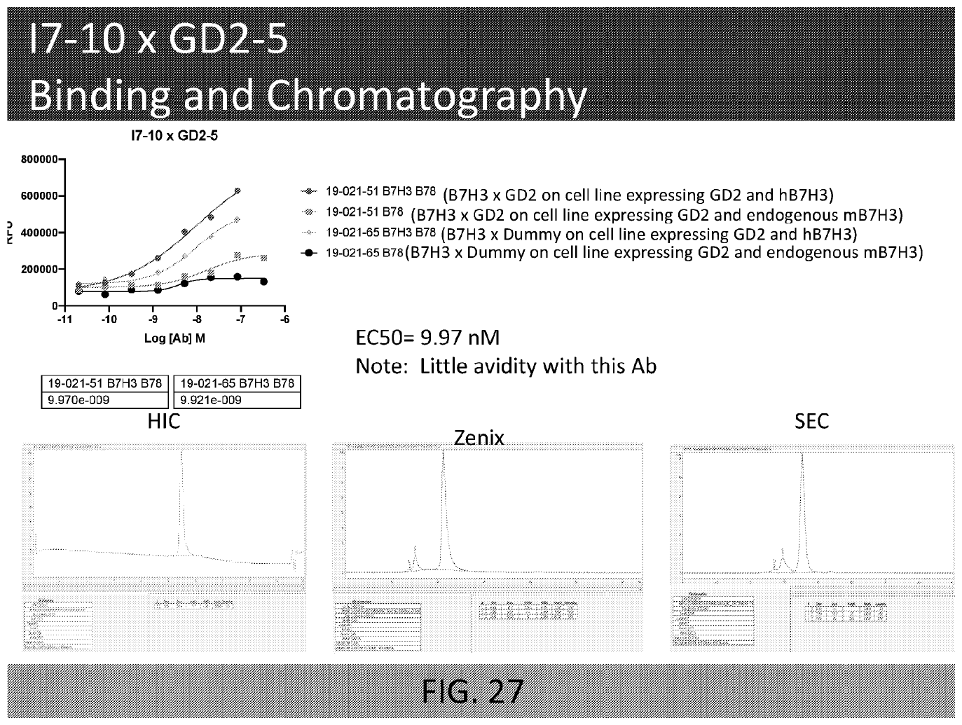

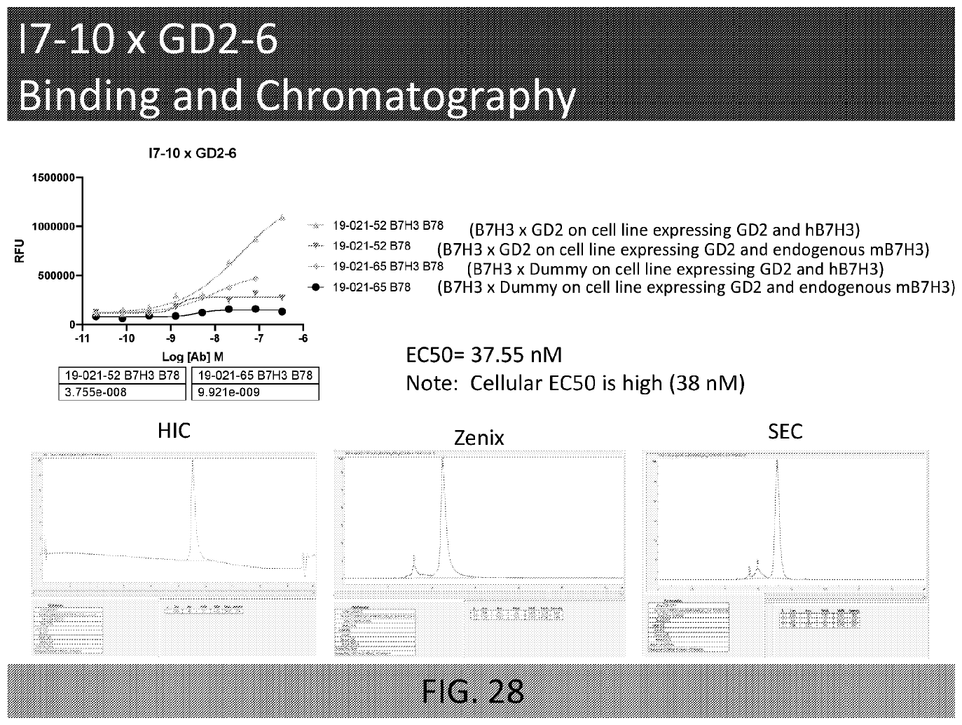

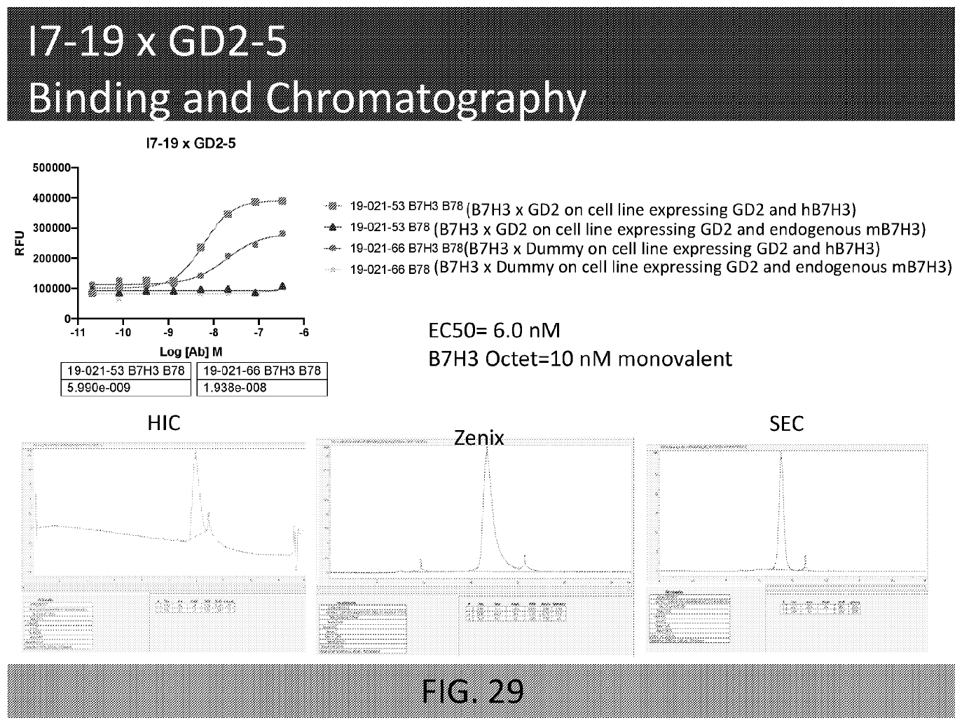

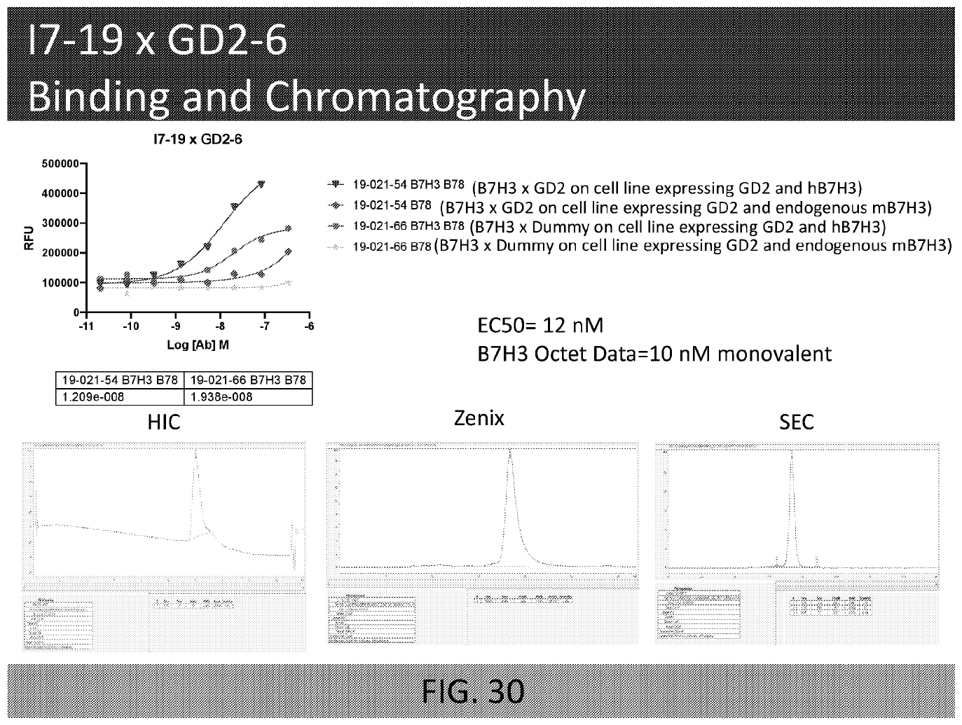

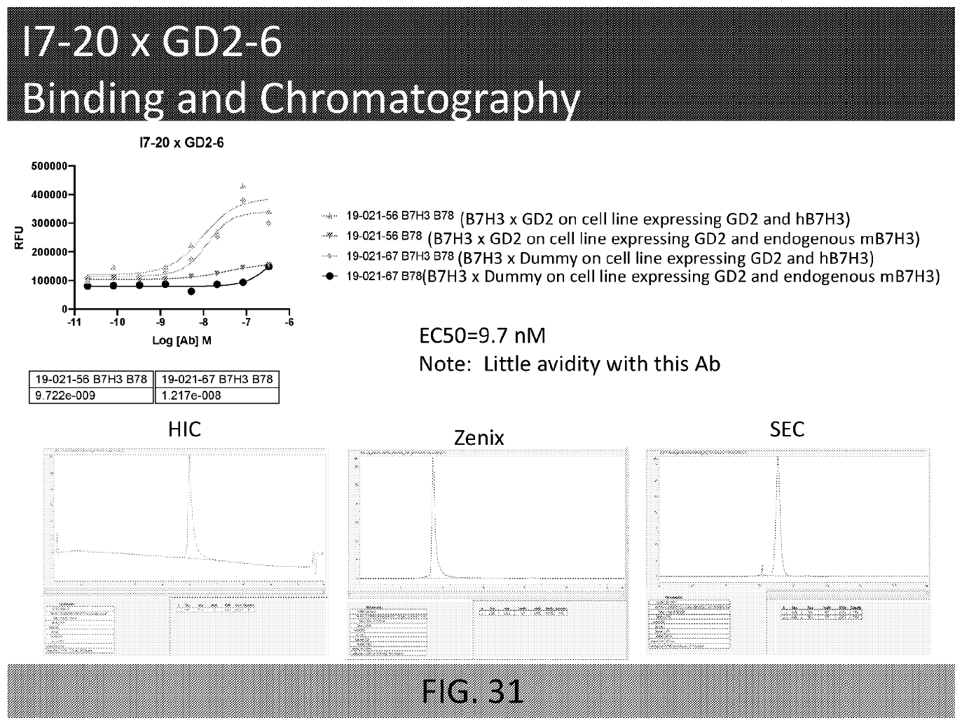

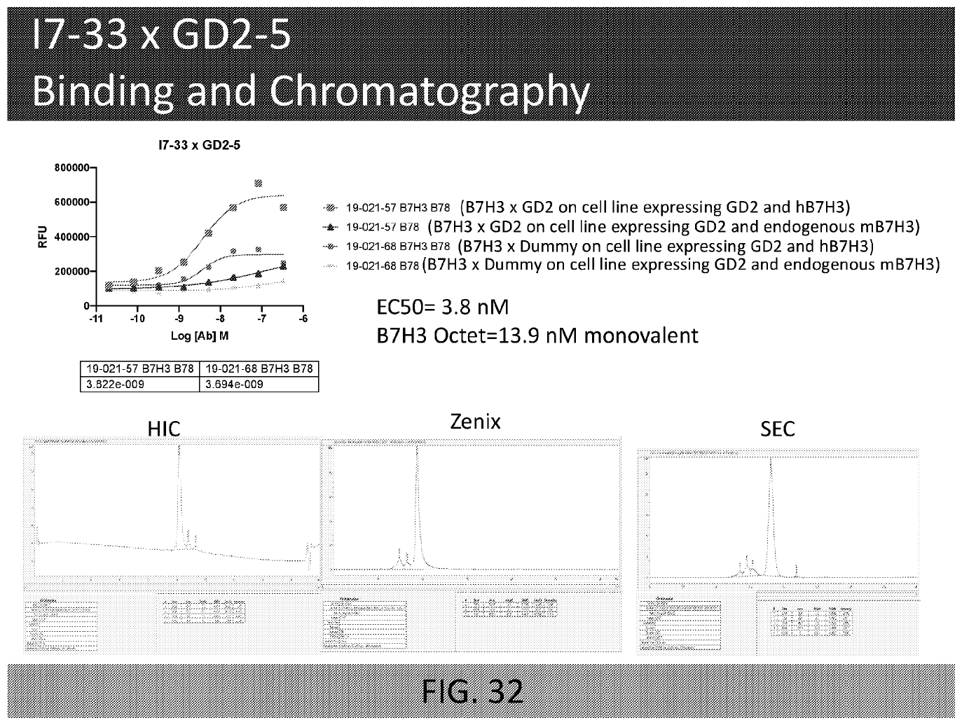

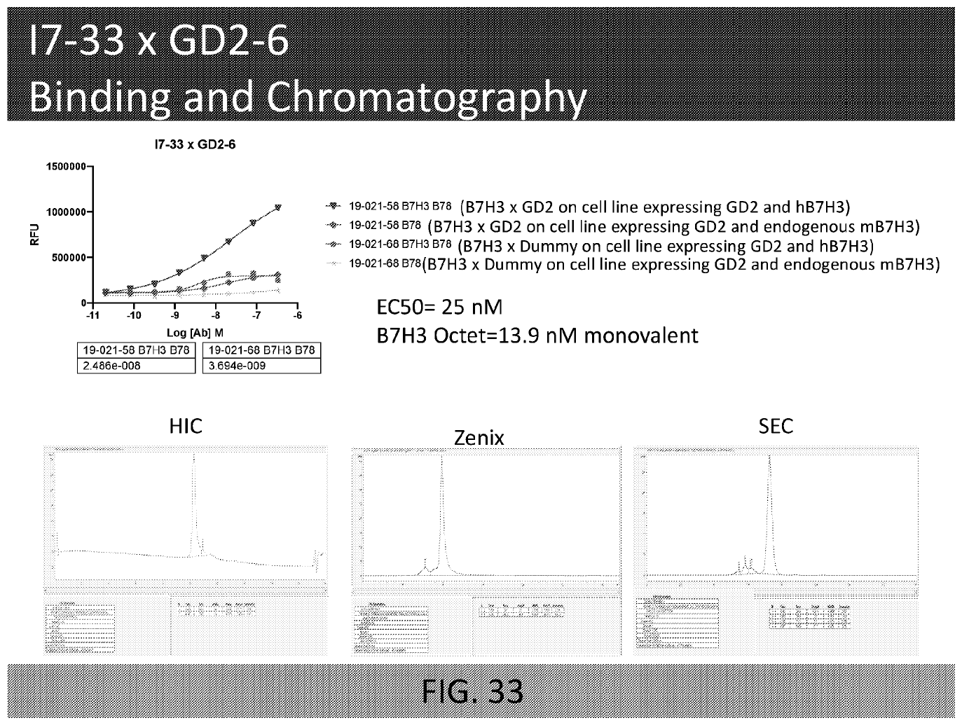

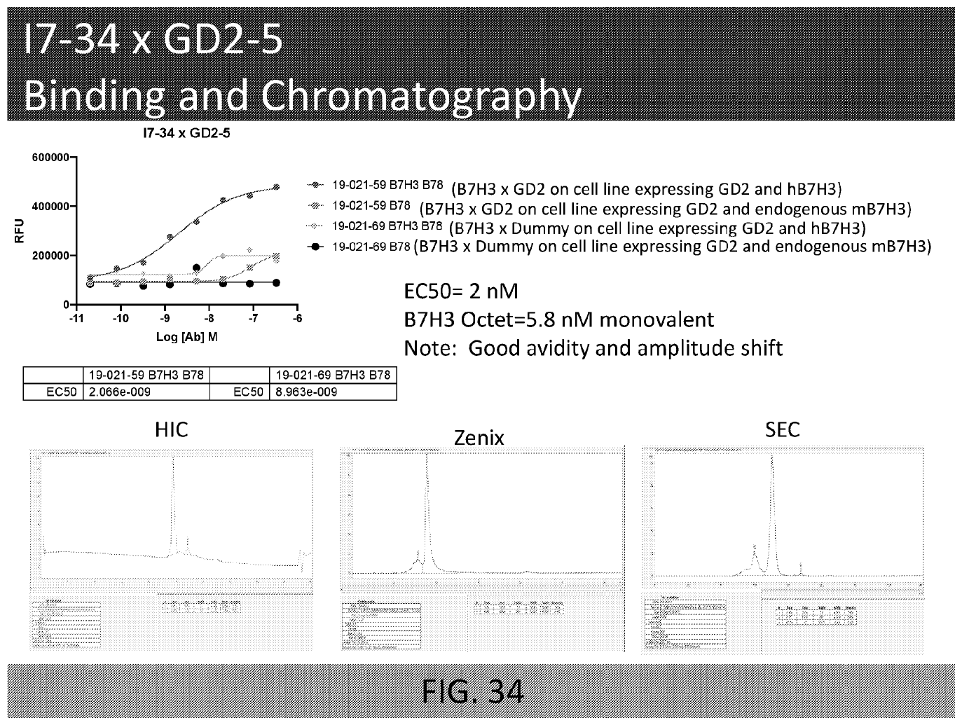

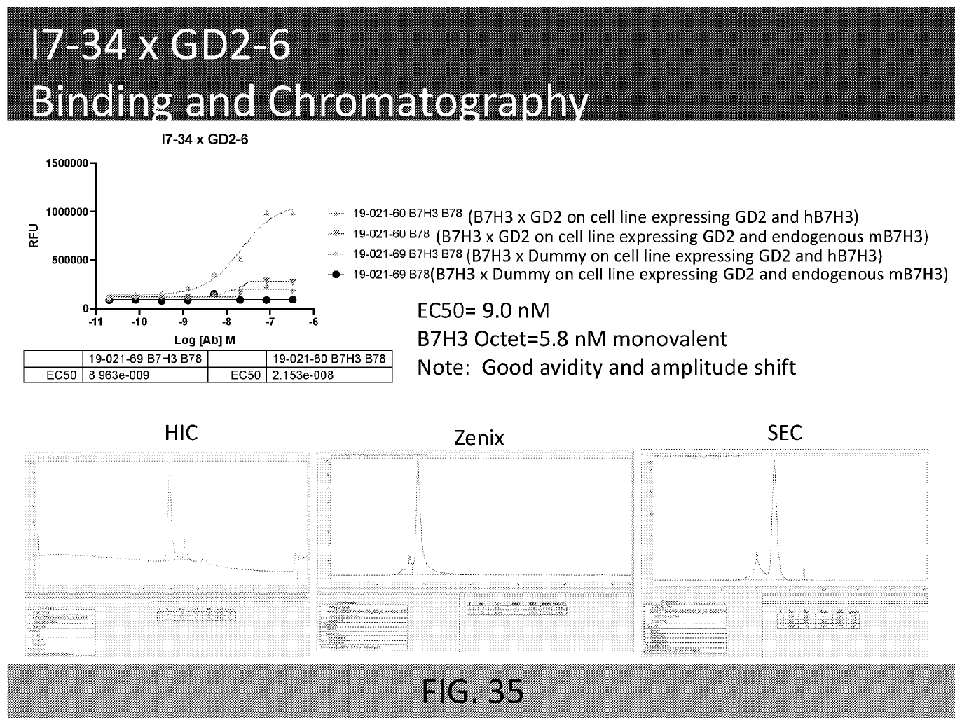

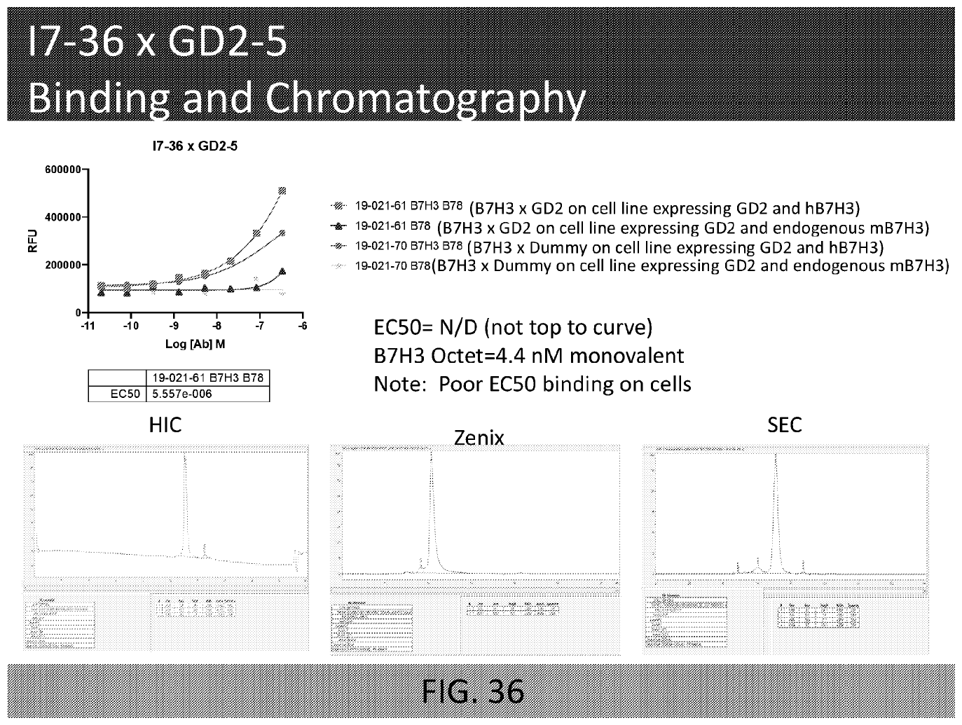

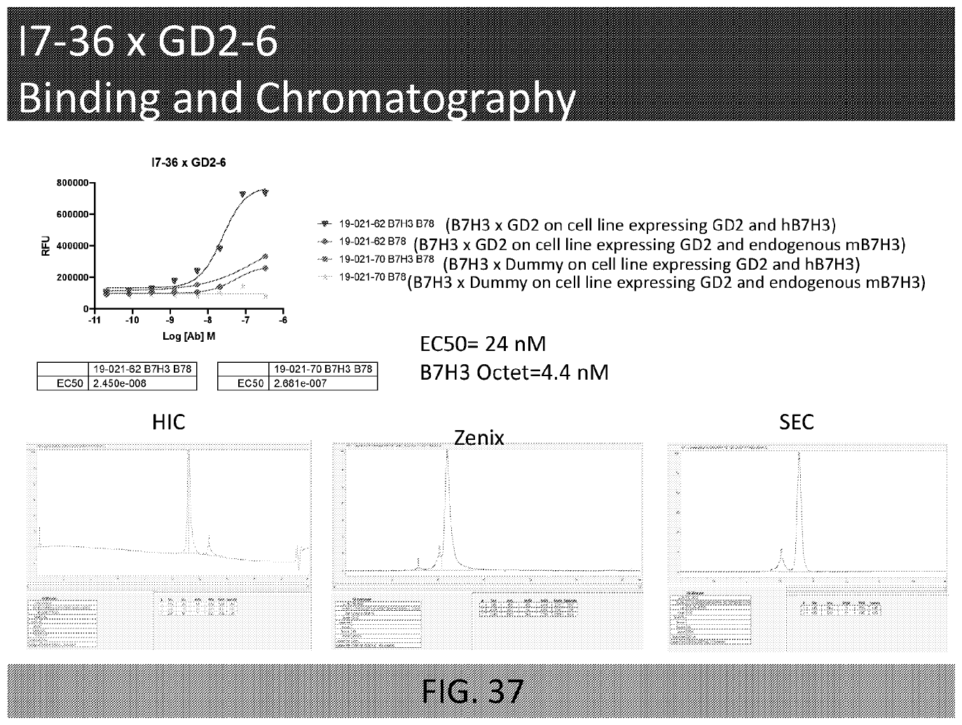

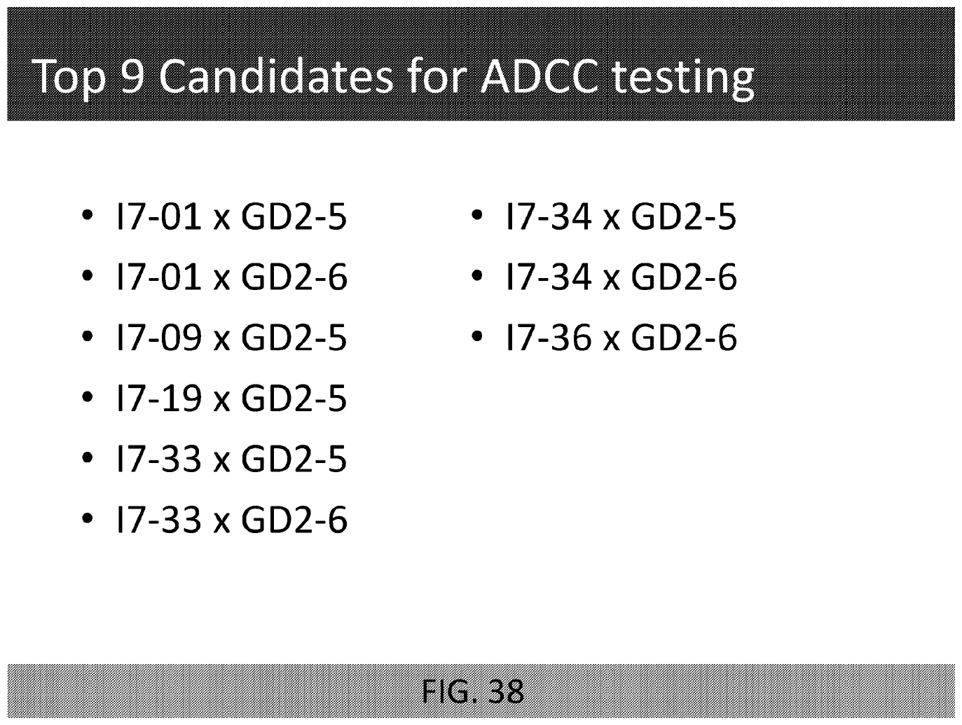

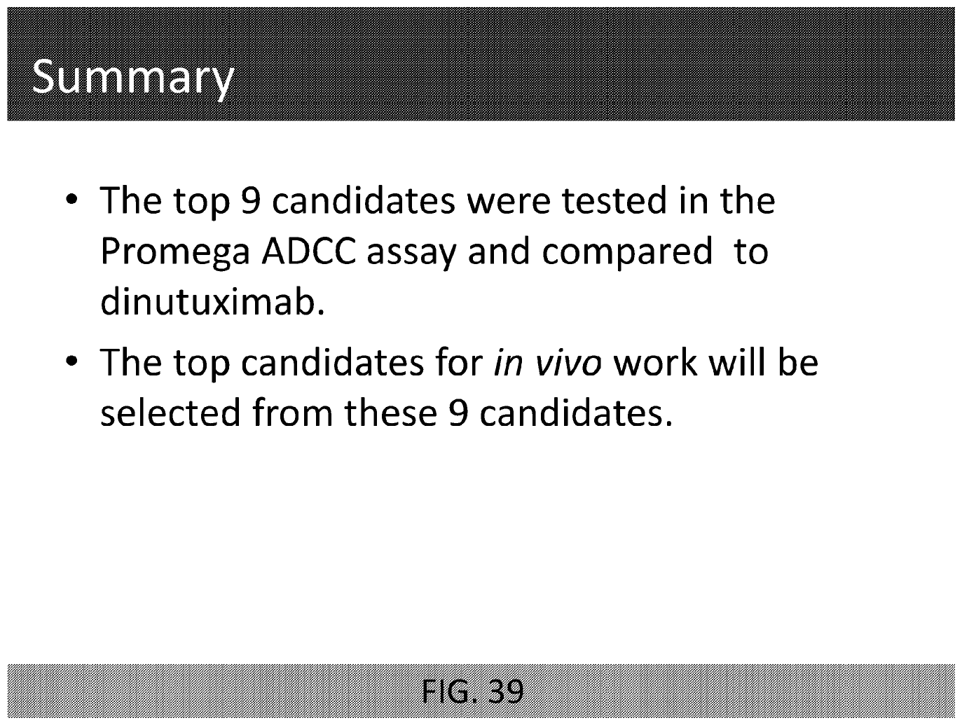

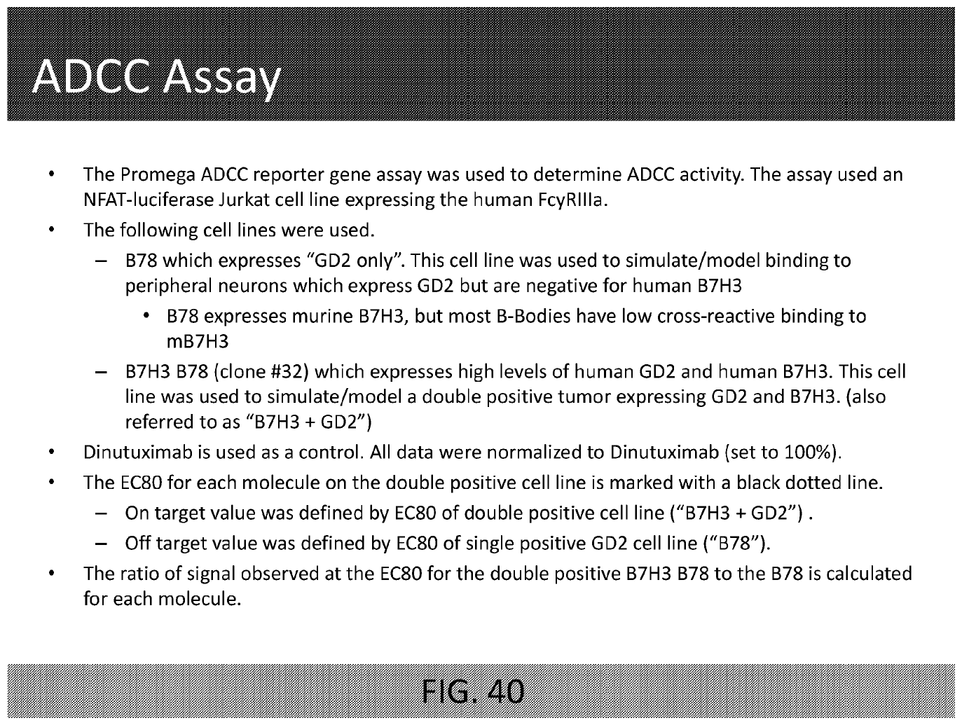

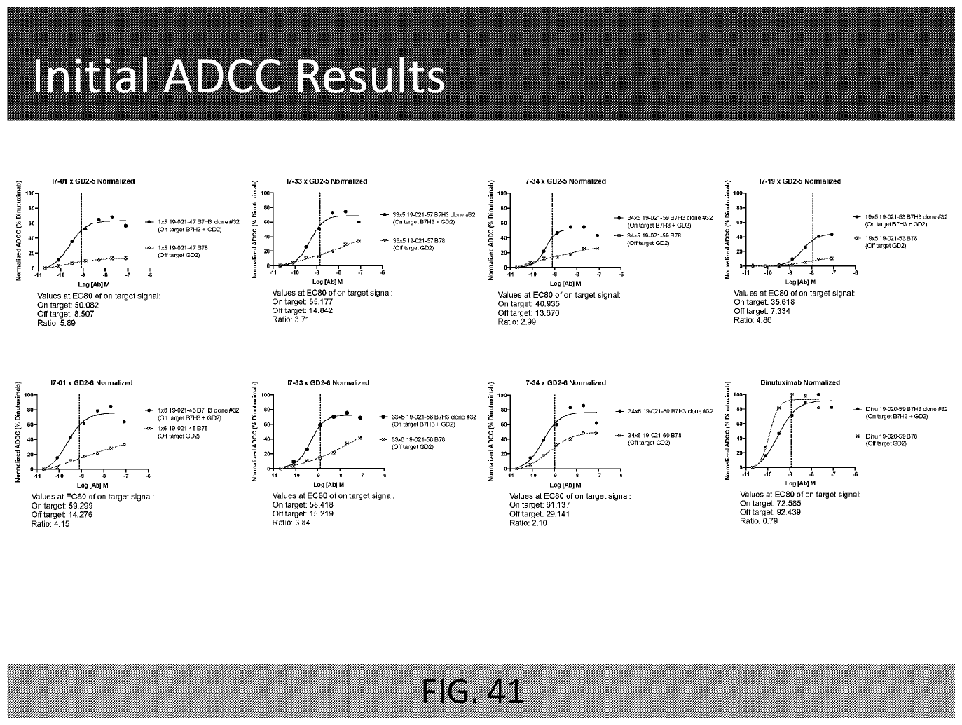

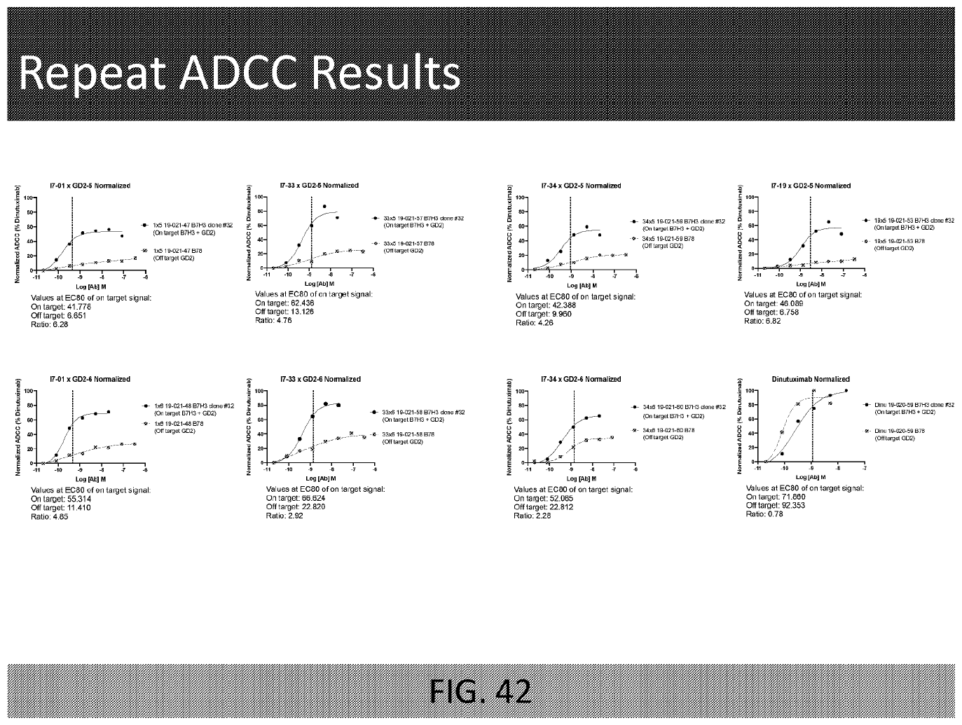

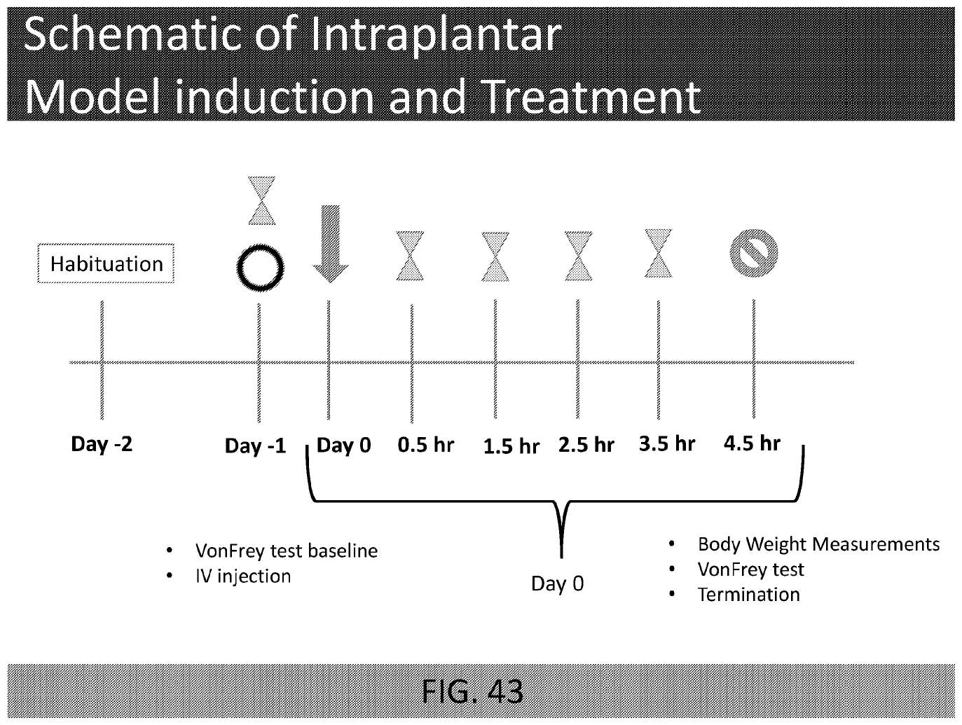

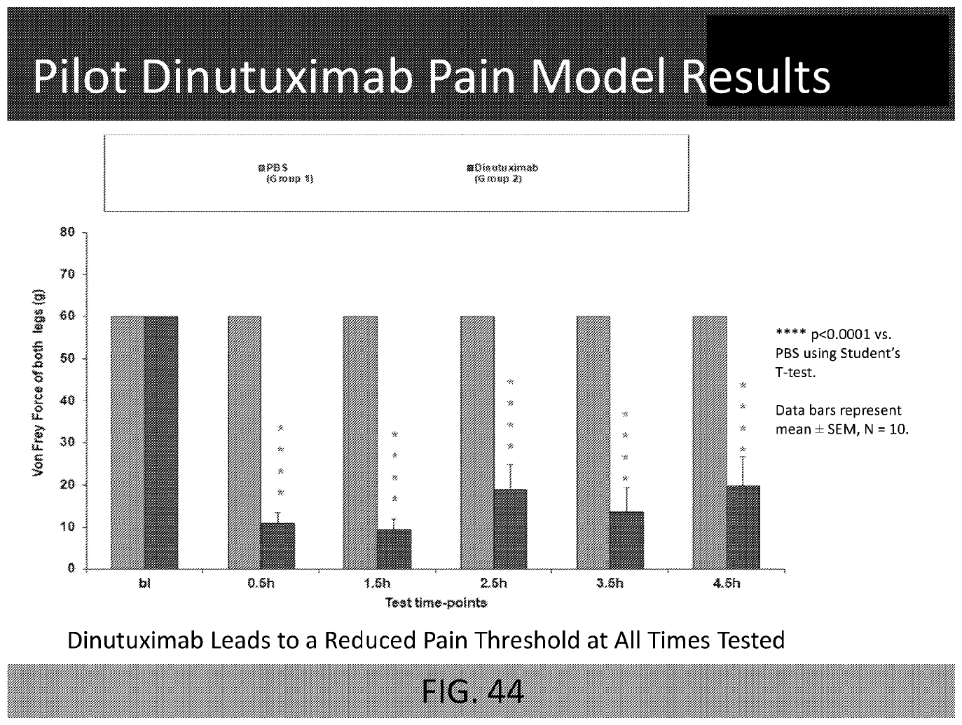

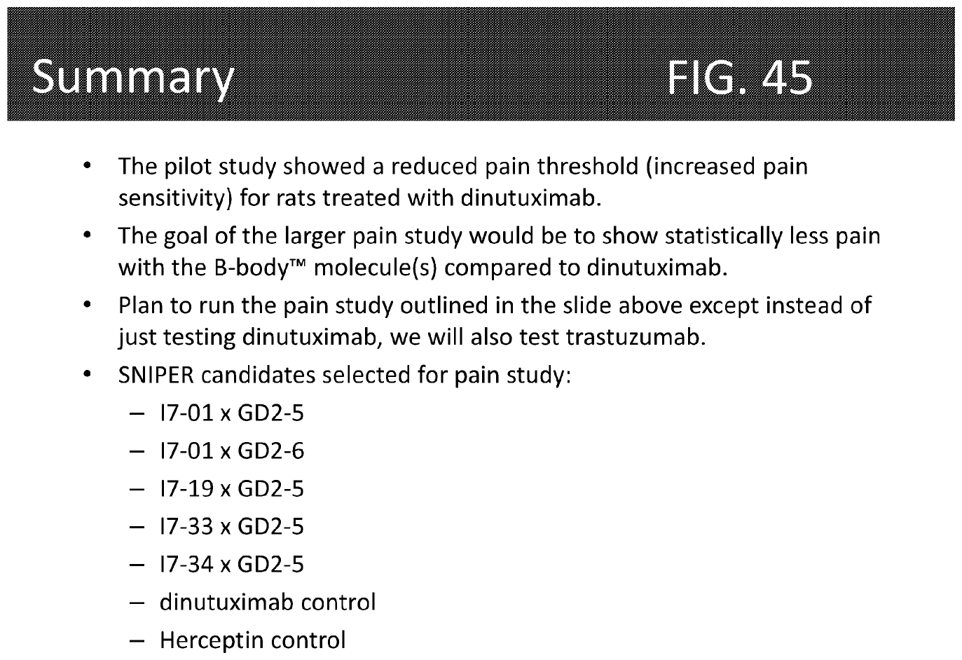

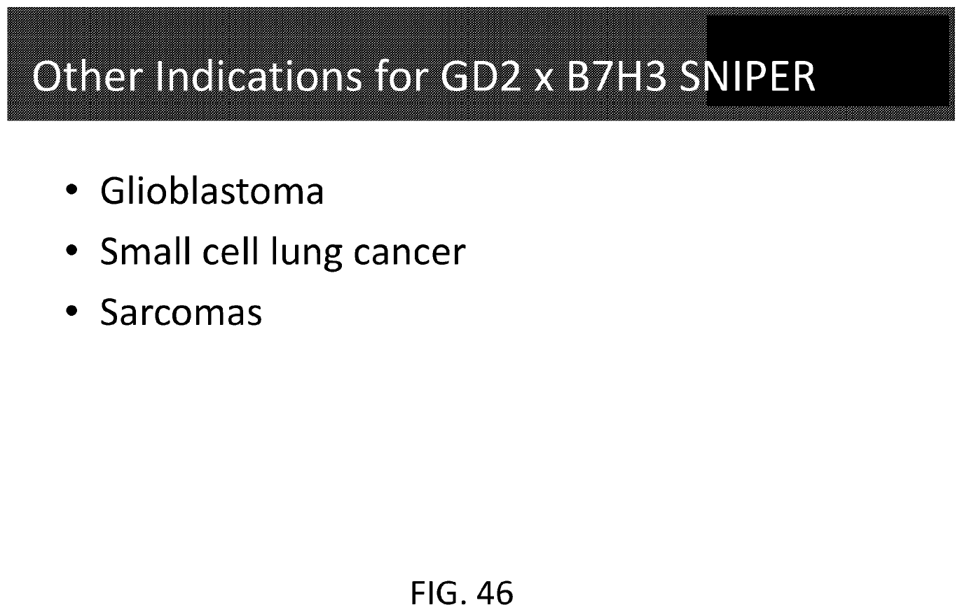

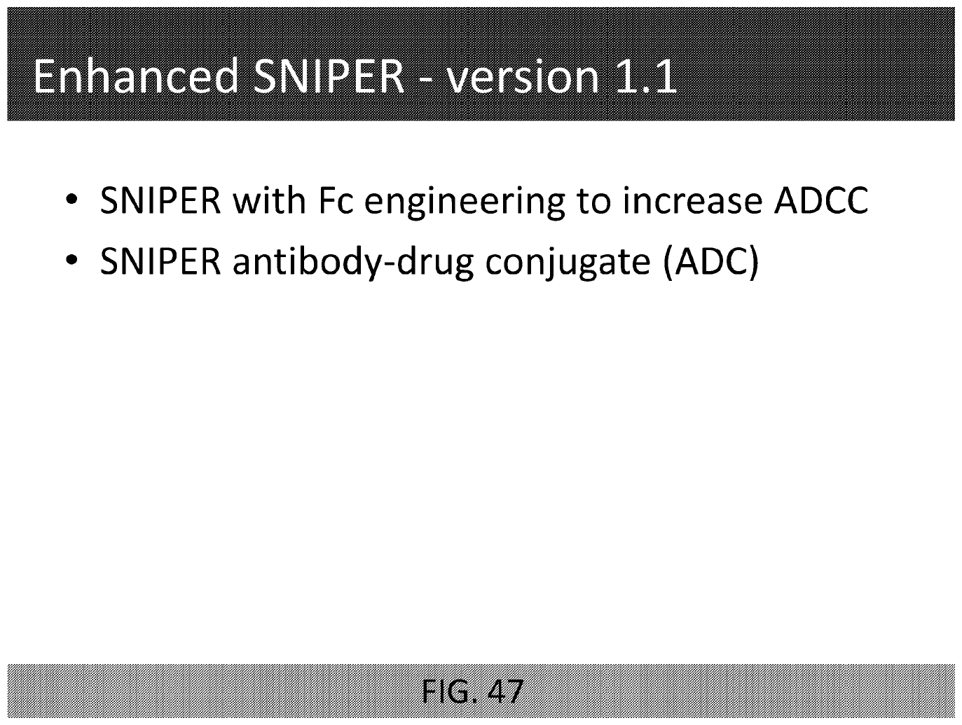

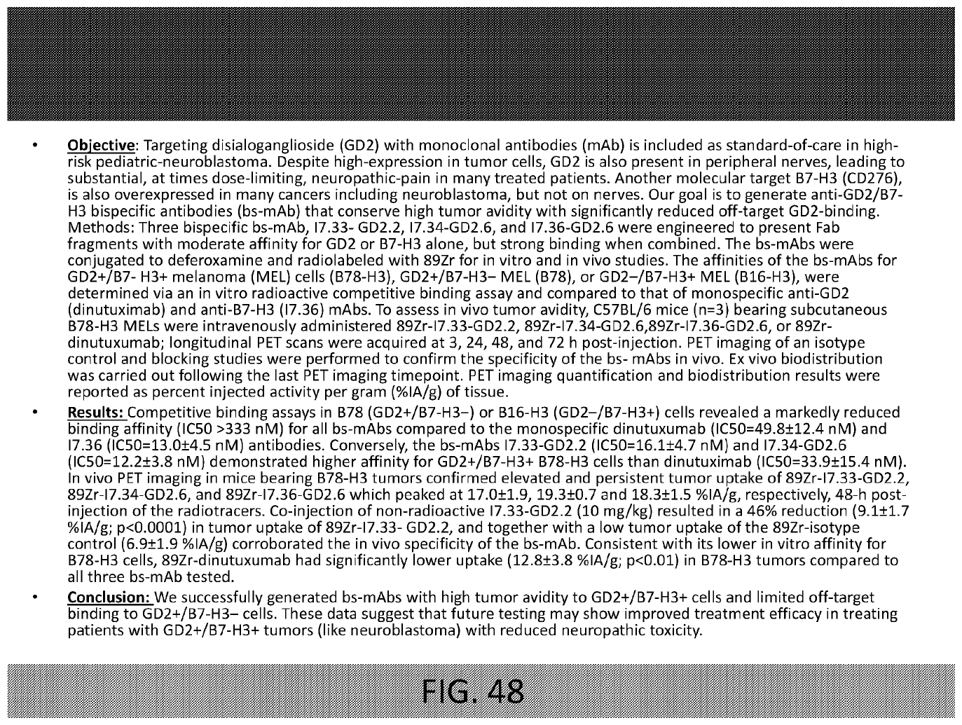

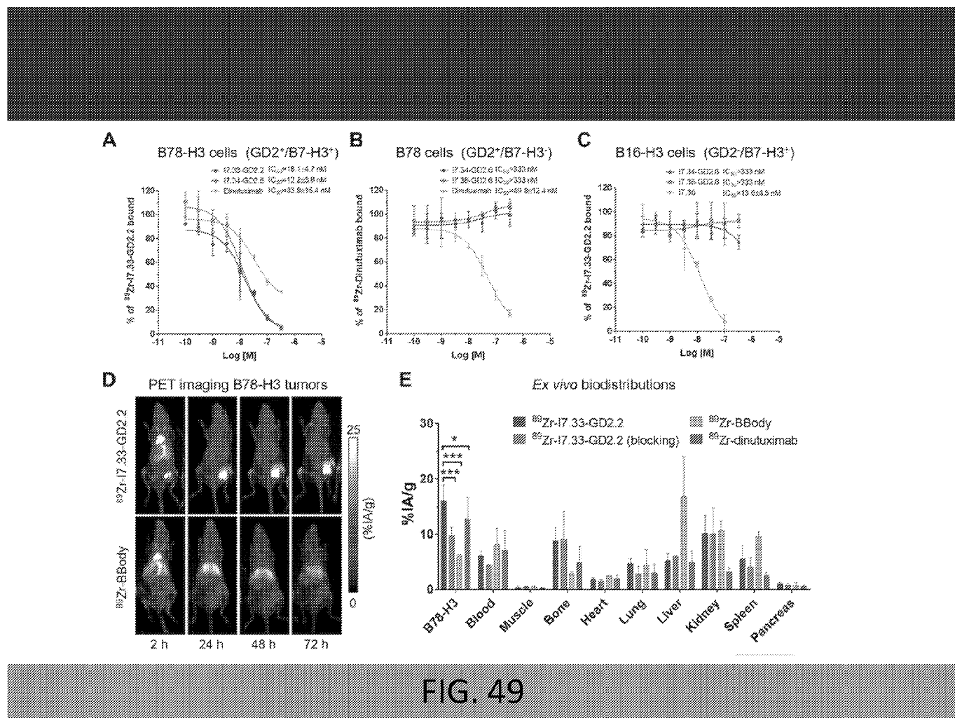

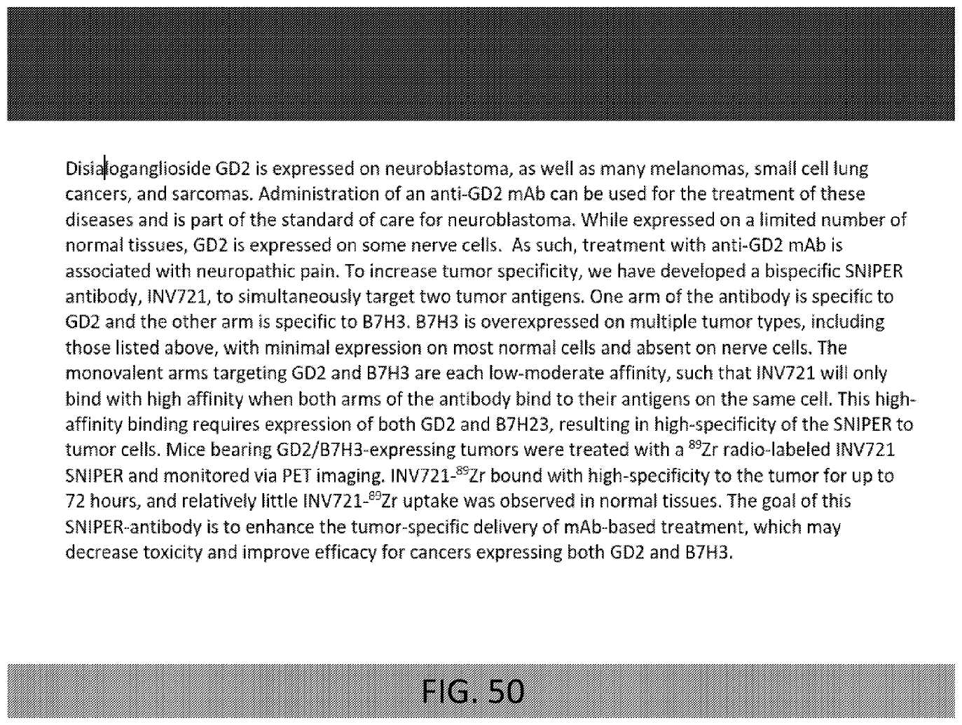

### 6. DETAILED DESCRIPTION OF THE INVENTION

### 6.1. DEFINITIONS

Unless otherwise defined herein, all technical and scientific terms used herein have the meaning commonly understood by a person skilled in the art to which this invention pertains.

As used herein, the following terms have the meanings ascribed to them below.

By “antigen binding site” (“ABS”) is meant a region of an antibody construct that specifically recognizes or binds to a given antigen or epitope. An ABS, and the antibody construct comprising such ABS, is said to “recognize” the epitope (or more generally, the antigen) to which the ABS specifically binds, and the epitope (or more generally, the antigen) is said to be the “recognition specificity” or “binding specificity” of the ABS

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g.)Octet/FORTEBIO®, surface plasmon resonance (SPR) technology (e.g.) Biacore®, and cell binding assays. Unless otherwise specified, for purposes herein affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10⁻¹⁰M.

The number of ABSs in an antibody construct as described herein defines the “valency” of the antibody construct. An antibody construct having a single ABS is “monovalent”. An antibody construct having a plurality of ABSs is said to be “multivalent”. A multivalent antibody construct having two ABSs is “bivalent.” A multivalent antibody construct three ABSs is “trivalent.” A multivalent antibody construct having four ABSs is “tetravalent.”

In various multivalent embodiments of antibody constructs, all of the plurality of ABSs have the same recognition specificity. Such a construct is a “monospecific” “multivalent” antibody construct. In other multivalent embodiments, at least two of the plurality of ABSs have different recognition specificities. Such antibody constructs are multivalent and “multispecific”. In multivalent embodiments in which the ABSs collectively have two recognition specificities, the binding molecule is “bispecific.” In multivalent embodiments in which the ABSs collectively have three recognition specificities, the binding molecule is “trispecific.”

In multivalent embodiments in which the ABSs collectively have a plurality of recognition specificities for different epitopes present on the same antigen, the antibody construct is “multiparatopic.” Multivalent embodiments in which the ABSs collectively recognize two epitopes on the same antigen are “biparatopic.”

In various multivalent embodiments, multivalency of the antibody construct improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABSs. Avidity can be measured by the same methods as those used to determine affinity, as described above. In certain embodiments, the avidity of a binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻¹⁰M. In certain embodiments, the avidity of a binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

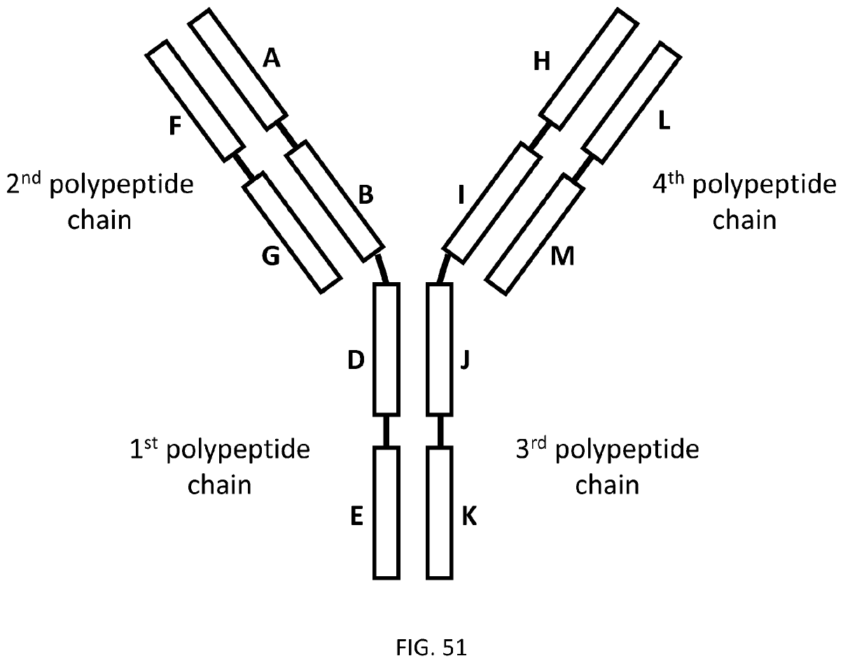

“B-Body,” as used herein, refers to antibody constructs as shown in FIG. 51, comprising a first and a second polypeptide chain, wherein: (a) the first polypeptide chain comprises a domain A, a domain B, a domain D, and a domain E, wherein the domains are arranged, from N-terminus to C-terminus, in a A-B-D-E orientation, and wherein domain A has a VL amino acid sequence, domain B has a CH3 amino acid sequence, domain D has a CH2 amino acid sequence, and domain E has a constant region domain amino acid sequence; (b) the second polypeptide chain comprises a domain F and a domain G, wherein the domains are arranged, from N-terminus to C-terminus, in a F-G orientation, and wherein domain F has a VH amino acid sequence and domain G has a CH3 amino acid sequence; and (c) the first and the second polypeptides are associated through an interaction between the A and the F domains and an interaction between the B and the G domains to form the antibody construct. B-bodies are described in more detail in US 2018/0118811 and WO 2019/204522, the disclosures of which are incorporated by reference in their entireties herein.

“Orthogonal modifications” or synonymously “orthogonal mutations” as described herein are one or more engineered mutations in an amino acid sequence of an antibody domain that alter the affinity of binding of a first domain having an orthogonal modification for a second domain having a complementary orthogonal modification, as compared to binding of the first and second domains in the absence of the orthogonal modifications. In some embodiments, the orthogonal modifications decrease the affinity of binding of the first domain having the orthogonal modification for the second domain having the complementary orthogonal modification, as compared to binding of the first and second domains in the absence of the orthogonal modifications. In some embodiments, the orthogonal modifications do not alter the affinity of binding of the first domain having the orthogonal modification for the second domain having the complementary orthogonal modification, as compared to binding of the first and second domains in the absence of the orthogonal modifications. In preferred embodiments, the orthogonal modifications increase the affinity of binding of the first domain having the orthogonal modification for the second domain having the complementary orthogonal modification, as compared to binding of the first and second domains in the absence of the orthogonal modifications. In certain preferred embodiments, the orthogonal modifications decrease the affinity of a domain having the orthogonal modifications for a domain lacking the complementary orthogonal modifications.

In particular embodiments, orthogonal modifications include, but are not limited to, engineered disulfide bridges, knob-in-hole mutations, and charge-pair mutations, as described in greater detail below. In particular embodiments, orthogonal modifications include a combination of orthogonal modifications selected from, but not limited to, engineered disulfide bridges, knob-in-hole mutations, and charge-pair mutations.

### 6.2. OTHER INTERPRETATIONAL CONVENTIONS

Unless otherwise specified, all references to “sequences” herein are to amino acid sequences. By “endogenous sequence” or “native sequence” is meant any sequence, including nucleic acid and amino acid sequences as context dictates, which originates from an organism, tissue, or cell and has not been artificially modified or mutated.

Unless otherwise specified, antibody constant region residue numbering is according to the Eu index as described at www. imgt. org/IMGTScientificChart/Numbering/Hu_IGHGnber.html which is hereby incorporated by reference in its entirety, and identifies the residue according to its location in an endogenous constant region sequence regardless of the residue's physical location within a chain of the antibody constructs described herein.

Unless otherwise specified, all references to complementarity determining regions (CDRs) are Kabat-defined CDRs.

The terms “first”, “second”, “third”, “fourth”, etc., when used with respect to polypeptide chains (e.g., a “first” polypeptide chain, a “second” polypeptide chain, etc. or polypeptide “chain 1,” “chain 2,” etc.) are used herein as a unique identifier for specific polypeptide chains that form a multimeric molecule, and are not intended to connote order or quantity of the different polypeptide chains within the antibody construct.

The terms “first”, “second”, “third”, “fourth”, etc., when used with respect to CH3 domains are used to designate specific domains, and are not intended to connote order or quantity of the domains.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or apparent from context, as used herein the term “or” is understood to be inclusive. Unless specifically stated or apparent from context, as used herein, the terms “a”, “an”, and “the” are understood to be singular or plural.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise specified, “about” means within 10% of the stated value.

### 6.3. ANTIBODY CONSTRUCTS

Disclosed herein are multi-specific antibody constructs that selectively bind to tumor cells surface antigens B7 Homolog 3 (B7H3), also known as Cluster of Differentiation 276 (CD276), and disialoganglioside (GD2). Preferably, the multi-specific antibody constructs selectively bind to tumor cells that express both B7H3 and GD2.

In some embodiments, the multi-specific antibody construct comprises a first antigen binding site (ABS) specific for a first tumor cell surface antigen and a second antigen binding site (ABS) specific for a second tumor cell surface antigen. In some embodiments, the first ABS exhibits a low binding affinity for the first tumor cell surface antigen. In some embodiments, the second ABS exhibits a low binding affinity for the second tumor cell surface antigen. In some embodiments, both the first and second ABSs exhibit low binding affinity for the first and second tumor cell surface antigens, respectively. Low binding affinity refers to binding with an equilibrium dissociation constant (K_{D}) that is higher than 10 nM, higher than 20 nM, preferably higher than 50 nM, more preferably higher than 100 nM, yet more preferably higher than 200 nM.

In some embodiments, the first ABS specifically binds the first tumor cell surface antigen with a K_{D} that is higher than 10 nM, higher than 20 nM, preferably higher than 50 nM, more preferably higher than 100 nM, yet more preferably higher than 200 nM. In some embodiments, the first ABS specifically binds the first tumor cell surface antigen with a K_{D}that is between about 10-1000 nM, preferably between about 50-900 nM, more preferably between about 100-800 nM, or yet even more preferably between about 200-500 nM.

In some embodiments, the second ABS specifically binds the second tumor cell surface antigen with a K_{D} that is higher than 10 nM, higher than 20 nM, preferably higher than 50 nM, more preferably higher than 100 nM, yet more preferably higher than 200 nM. In some embodiments, the second ABS specifically binds the second tumor cell surface antigen with a K_{D} that is between about 10-1000 nM, preferably between about 50-900 nM, more preferably between about 100-800 nM, or yet even more preferably between about 200-500 nM.

In some embodiments, the first and second ABS's do not exhibit appreciable binding affinity to any other antigen. In some embodiments, the first and second ABS's exhibit a K_{D} to a non-target antigen that is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more than 500× higher than their K_{D} for the first or second tumor cell surface antigens, respectively.

In particular embodiments, the first ABS specifically binds B7H3 with a K_{D} that is between 5-200 nM, preferably between about 10-100 nM, more preferably between about 20-60 nM, or yet even more preferably between about 20-40 nM.

In particular embodiments, the second ABS specifically binds GD2 with a K_{D} that is between about 50-500 nM, preferably between about 100-400 nM, more preferably between about 100-300 nM, or yet even more preferably between about 150-250 nM.

In some embodiments, the multi-specific antibody construct binds to a target tumor cell that expresses B7H3 and GD2 with high avidity. In some embodiments, the multi-specific antibody construct binds to a target tumor cell with a K_{D} that is less than about 500 nM, less than about 400 nM, less than about 300 nM, less than about 200 nM, less than about 100 nM, less than about 90 nM, less than about 80 nM, less than about 70 nM, less than about 60 nM, preferably less than about 50 nM, more preferably less than about 25 nM, or even more preferably less than about 10 nM. For example, in some embodiments, the multi-specific antibody construct binds to a target tumor cell with a K_{D} that is less than about 9 nM, less than about 8 nM, less than about 7 nM, less than about 6 nM, less than about 5 nM, less than about 4 nM, less than about 3 nM, less than about 2 nM, or less than about 1 nM.

The multi-specific antibody construct may specifically bind to a target tumor cell with a higher avidity than the individual binding affinities of its ABS's for the first and second tumor cell surface antigens. For example, the multi-specific antibody construct may exhibit a K_{D} for the first or second tumor cell surface antigen that is higher than the antibody construct's K_{D} for the target tumor cell. In some embodiments, the multi-specific antibody construct exhibits a K_{D} for the first and second tumor cell surface antigens that is at least 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or more than 100× higher than the K_{D} of the antibody construct to the target tumor cell.

In some embodiments, the first ABS binds to the first tumor cell surface antigen with a K_{D} that is greater than 100 nM, the second ABS binds to the second tumor cell surface antigen with a K_{D} that is greater than 100 nM, and the multi-specific antibody construct binds to a target tumor cell with a Ku that is less than 10 nM.

In some embodiments, the first ABS binds to the first tumor cell surface antigen with a K_{D} that is greater than 100 nM, the second ABS binds to the second tumor cell surface antigen with a K_{D} that is greater than 100 nM, and the multi-specific antibody construct binds to a target tumor cell with a Ku that is less than 8 nM.

In some embodiments, the first ABS binds to the first tumor cell surface antigen with a K_{D} that is greater than 100 nM, the second ABS binds to the second tumor cell surface antigen with a K_{D} that is greater than 100 nM, and the multi-specific antibody construct binds to a target tumor cell with a K_{D} that is less than 5 nM.

In some embodiments, the multi-specific antibody construct specifically binds to a target tumor cell with a greater avidity than to any non-target cell. For example, the multi-specific antibody construct may bind to a target tumor cell with an avidity that is at least 0.1×, 0.2×, 0.3×, 0.4×, 0.5×, 0.6×, 0.7×, 0.8×, 0.9×, 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 750×, or 1000× greater than its avidity for a non-target cell.

The multi-specific antibody construct may selectively bind to a target tumor cell over non-target cells. A skilled artisan may assess selective binding to the target tumor cell over non-target cells using any methods known in the art. An exemplary method for assessing selective binding comprises comparing a percentage of target tumor cells which are detectably labeled with the multi-specific antibody construct under non-saturating assay conditions to a percentage of non-target cells which are detectably labeled with the multi-specific antibody construct under the same assay conditions. For example, a ratio of the percent target tumor cells bound/percent non-target cells bound by the multi-specific antibody construct may be used as an indication of selective binding to the target tumor cell. In some embodiments, a multi-specific antibody construct that detectably binds over 70% of target tumor cells under non-saturating assay conditions binds less than 30%, less than 25%, less than 20%, or less than 15% of non-target cells under the same assay conditions. In some embodiments, a multi-specific antibody construct that detectably binds over 80% of target tumor cells under non-saturating assay conditions binds less than 20% of non-target cells under the same assay conditions. In some embodiments, a multi-specific antibody construct that detectably binds over 90% of target tumor cells under non-saturating assay conditions binds less than 10% of non-target cells under the same assay conditions. In some embodiments, the ratio of bound target tumor cells/bound non-target cells under non-saturating assay conditions is greater than 1.5, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 110, greater than 120, greater than 130, greater than 140, greater than 150, greater than 160, greater than 170, greater than 180, greater than 190, greater than 200, greater than 210, greater than 220, greater than 230, greater than 240, greater than 250, greater than 260, greater than 270, greater than 280, greater than 290, greater than 300, greater than 310, greater than 320, greater than 330, greater than 340, greater than 350, greater than 360, greater than 370, greater than 380, greater than 390, greater than 400, greater than 410, greater than 420, greater than 430, greater than 440, greater than 450, greater than 460, greater than 470, greater than 480, greater than 490, or greater than 500.

Target tumor cells are distinguished from non-target cells based on co-expression of B7H3 and GD2. Accordingly, target tumor cells are double positive for B7H3 and GD2. Non-target cells include, but are not limited to, cells that express B7H3 but do not express GD2 and cell that express GD2 but do not express B7H3.

### 6.3.1. Variable Domains

In typical embodiments, each ABS of the antibody constructs described herein comprises a V_{H} domain and a V_{L} domain. In preferred embodiments, the V_{L} domain of the first polypeptide and the V_{H} domain of the second polypeptide associate to form a first ABS. In further preferred embodiments, the V_{L} domain of the third polypeptide and the V_{H} domain of the fourth polypeptide associate to form a second ABS.

In some embodiments, with reference to FIG. 51, the first polypeptide and the third polypeptide of the antibody construct both comprise a V_{H} domain. In some embodiments, the second polypeptide and the fourth polypeptide of the antibody construct both comprise a V_{L} domain.

In particular embodiments, with reference to FIG. 51, the domains of the polypeptide chain comprising the V_{L} domain and the polypeptide chain comprising the V_{H} domain are respectively ordered, from N terminus to C terminus:

a) first polypeptide chain

| domain A | domain B | domain D | domain E |
|---|---|---|---|
| V_{L} | first CH3 | CH2 | third CH3 |

second polypeptide chain

| domain F | domain G |
|---|---|
| V_{H} | second CH3 |

or

b) first polypeptide chain

| domain A | domain B | domain D | domain E |
|---|---|---|---|
| V_{L} | second CH3 | CH2 | third CH3 |

second polypeptide chain

| domain F | domain G |
|---|---|
| V_{H} | first CH3. |

In various embodiments, with reference to FIG. 51, the antibody construct further comprises a third polypeptide chain and a fourth polypeptide chain, wherein:

(a) the third polypeptide chain comprises a domain H, a domain I, a domain J, and a domain K, wherein the domains are arranged, from N-terminus to C-terminus, in a H-I-J-K orientation, and wherein domain H has a variable region domain amino acid sequence, domain I has a constant region domain amino acid sequence, domain J has a CH2 amino acid sequence, and K has a constant region domain amino acid sequence;

(b) the fourth polypeptide chain comprises a domain L and a domain M, wherein the domains are arranged, from N-terminus to C-terminus, in a L-M orientation, and wherein domain L has a variable region domain amino acid sequence and domain M has a constant region domain amino acid sequence;

(c) the third and the fourth polypeptides are associated through an interaction between the H and the L domains, which form a second antigen binding site (ABS) and an interaction between the I and the M domains; and

(d) the first and the third polypeptides are associated through an interaction between the D and the J domains and an interaction between the E and the K domains.

In particular embodiments, domain H is a V_{L} domain; domain I is a C_{L} domain; domain J is a CH2 domain; and domain K is a fourth CH3 domain. In specific embodiments, domain L is a V_{H} domain; and domain M is a CH1 domain.

In particular embodiments, domain A has a VL antibody domain sequence and domain F has a VH antibody domain sequence. In some embodiments, domain A has a VH antibody domain sequence and domain F has a VL antibody domain sequence.

In some embodiments, the antibody construct comprises a native antibody architecture, wherein domains A and H comprise VH amino acid sequences, domains F and L comprise VL amino acid sequences, domains B and I comprise CH1, domains G and M comprise CL, domains D and J comprise CH2, and domains E and K comprise CH3.

In some embodiments, the antibody construct is a B-Body™. B-Body™ antibody constructs are described in US 2018/0118811 and WO 2019/204522, the disclosures of which are incorporated herein by reference in their entireties, with specific embodiments further described below.

In some embodiments, the antibody construct is a CrossMab™. CrossMab™ antibodies are described in U.S. Pat. Nos. 8,242,247; 9,266,967; and 8,227,577, U.S. Patent Application Pub. No. 20120237506, U.S. Patent Application Pub. No. US20090162359, WO2016016299, WO2015052230. In some embodiments, the antibody construct is a bivalent, bispecific antibody, comprising: a) the light chain and heavy chain of an antibody specifically binding to a first antigen; and b) the light chain and heavy chain of an antibody specifically binding to a second antigen, wherein constant domains CL and CH1 from the antibody specifically binding to a second antigen are replaced by each other.

In some embodiments, the antibody construct is an antibody having a general architecture described in U.S. Pat. No. 8,871,912 and WO 2016/087650. In some embodiments, the antibody construct is a domain-exchanged antibody comprising a light chain (LC) composed of VL-CH3, and a heavy chain (HC) comprising VH-CH3-CH2-CH3, wherein the VL-CH3 of the LC dimerizes with the VH-CH3 of the HC thereby forming a domain-exchanged LC/HC dimer comprising a CH3LC/CH3HC domain pair.

In some embodiments, the antibody construct is as described in WO 2017/011342. In some embodiments, the antibody construct is as described in WO 2006/093794.

### 6.3.1.1.1 VH Domains

In various embodiments, the VH domain has an amino acid sequence that is a mammalian sequence, including human sequences, humanized sequences, synthetic sequences, or combinations of human, non-human mammalian, and/or synthetic sequences. In various embodiments, the VH amino acid sequences are mutated sequences of naturally occurring sequences.

### 6.3.1.1.2 VL Domains

In various embodiments, the VL domain has an amino acid sequence that is a mammalian sequence, including human sequences, humanized sequences, synthetic sequences, or combinations of human, non-human mammalian, and/or synthetic sequences. In various embodiments, VL amino acid sequences are mutated sequences of naturally occurring sequences.

In certain embodiments, the VL amino acid sequence is a lambda (λ) light chain variable domain sequence. In certain embodiments, the VL amino acid sequence is a kappa (κ) light chain variable domain sequence.

### 6.3.1.1.3 Complementarity Determining Regions

The VH and VL domains of the antibody constructs described herein comprise highly variable sequences termed “complementarity determining regions” (CDRs), typically three CDRs (CDR1, CD2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries.

### 6.3.1.1.4 Framework Regions and CDR Grafting

The VH and VL domains of the antibody constructs described herein comprise “framework region” (FR) sequences. FRs are generally conserved sequence regions that act as a scaffold for interspersed CDRs, typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, canine, feline, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthetic sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.3.2. CH1 and CL Domains

In various embodiments, at least one of the first and second polypeptide chains of the antibody construct further comprises an immunoglobulin CH1 domain. In various embodiments, at least one of the first and second polypeptide chains of the antibody construct further comprises an immunoglobulin C_{L} domain.

In various embodiments, the first polypeptide further comprises a CH1 domain and the second polypeptide further comprises a CL domain. In various embodiments, the second polypeptide further comprises a CH1 domain and the first polypeptide further comprises a CL domain.

In various embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The C_{L} domains of the antibody constructs described herein are antibody light chain constant domains. In certain embodiments, the C_{L} domains have sequences that are endogenous sequences. In a variety of embodiments, the CL sequences are mammalian sequences, including, but not limited to mouse, rat, hamster, rabbit, canine, feline, camel, donkey, goat, and human sequences. In a preferred embodiment, CL sequences are human sequences.

In certain embodiments, the CL amino acid sequences are lambda (λ) light chain constant domain sequences. In particular embodiments, the CL amino acid sequences are human lambda light chain constant domain sequences. In preferred embodiments, the lambda (λ) light chain sequence is UniProt accession number POCG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences.

The CH1 domain folding is typically the rate limiting step in the secretion of IgG (Feige et al. Mol Cell. 2009 Jun. 12; 34(5):569-79; herein incorporated by reference in its entirety). Thus, purifying the antibody constructs described herein based on the rate limiting component of CH1-comprising polypeptide chains can provide a means to purify complete complexes from incomplete chains, e.g., purifying complexes having a limiting CH1 domain from complexes only having one or more non-CH1 comprising chains.

While the CH1 limiting expression may be a benefit in some aspects, as discussed, there is the potential for CH1 to limit overall expression of the complete antibody constructs. Thus, in certain embodiments, the expression of the polypeptide chain comprising the CH1 sequence(s) is adjusted to improve the efficiency of the antibody constructs forming complete complexes. In an illustrative example, the ratio of a plasmid vector constructed to express the polypeptide chain comprising the CH1 sequence(s) can be increased relative to the plasmid vectors constructed to express the other polypeptide chains. In another illustrative example, the polypeptide chain comprising the CH1 sequence(s) when compared to the polypeptide chain comprising the CL sequence(s) can be the smaller of the two polypeptide chains. In another specific embodiment, the expression of the polypeptide chain comprising the CH1 sequence(s) can be adjusted by controlling which polypeptide chain has the CH1 sequence(s). For example, engineering the antibody construct such that the CH1 domain is present in a two-domain polypeptide chain (e.g., the 4th polypeptide chain described herein), instead of the CH1 sequence's native position in a four-domain polypeptide chain (e.g., the 3rd polypeptide chain described herein), can be used to control the expression of the polypeptide chain comprising the CH1 sequence(s). However, in other aspects, a relative expression level of CH1 containing chains that is too high compared to the other chains can result in incomplete complexes the have the CH1 chain, but not each of the other chains. Thus, in certain embodiments, the expression of the polypeptide chain comprising the CH1 sequence(s) is adjusted to both reduce the formation incomplete complexes without the CH1 containing chain, and to reduce the formation incomplete complexes with the CH1 containing chain but without the other chains present in a complete complex.

In certain embodiments, the CH1 sequence and the CL sequences separately comprise respectively orthogonal modifications in endogenous CH1 and CL sequences, as discussed in greater detail below. Preferably, the orthogonal mutations in the CH1 sequence do not eliminate the specific binding interaction between the CH1 domain and CH1-specific binding reagent used for purification.

### 6.3.2.1 CH1 and CL Orthogonal Modifications

In certain embodiments, the CH1 sequence and the CL sequences further comprise respectively orthogonal modifications of endogenous CH1 and CL sequences. A CH1/CL orthogonal modification may affect the CH1/C_{L} domain pairing via an interaction between a modified residue in the CH1 domain and a corresponding modified or unmodified residue in the C_{L} domain.

In particular embodiments, the orthogonal modifications can be combined with amino acid substitutions that reduce immunogenicity, such as isoallotype mutations.

In other embodiments, one sequence of the CH1/CL pair comprises at least one modification while the other sequence of the CH1/CL pair does not comprise a modification in the respectively orthogonal amino acid position.

CH1 and CL sequences can also be portions thereof, either of an endogenous or modified sequence, such that a domain having the CH1 sequence, or portion thereof, can associate with a domain having the CH1 sequence, or portion thereof. Furthermore, the antibody construct having a portion of the CH1 sequences described herein can be bound by a CH1 binding reagent.

### 6.3.2.1.1 Exemplary CH1/CL Orthogonal Modifications: Engineered Disulfide Bridges

Some embodiments of a CH1/CL orthogonal modification comprise an engineered disulfide bridge between engineered cysteines in CH1 and CL. Such engineered disulfide bridges may stabilize an interaction between the polypeptide comprising the modified CH1 and the polypeptide comprising the corresponding modified CL.

An orthogonal CH1/CL modification comprising an engineered disulfide bridge can comprise, by way of example only, a CH1 domain having an engineered cysteine at position 128, 129, 138, 141, 168, or 171, as numbered by the EU index. Such an orthogonal CH1/CL modification comprising an engineered disulfide bridge may further comprise, by way of example only, a C_{L} domain having an engineered cysteine at position 116, 118, 119, 164, 162, or 210, as numbered by the EU index.

For example, a CH1/CL orthogonal modification may be selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In some embodiments, the CH1/CL orthogonal modification comprises an engineered cysteine at position 141 of the CH1 sequence and position 118 of the CL sequence, as numbered by the EU index.

In some embodiments, the CH1/CL orthogonal modification comprises an engineered cysteine at position 168 of the CH1 sequence and position 164 of the CL sequence, as numbered by the EU index. In some embodiments, the CH1/CL orthogonal modification comprises an engineered cysteine at position 128 of the CH1 sequence and position 118 of the CL sequence, as numbered by the EU index. In some embodiments, the CH1/CL orthogonal modification comprises an engineered cysteine at position 171 of the CH1 sequence and position 162 of the CL sequence, as numbered by the EU index. In some embodiments, the CL sequence is a CL-lambda sequence. In preferred embodiments, the CL sequence is a CL-kappa sequence. In some embodiments, the engineered cysteines are at position 128 of the CH1 sequence and position 118 of the CL Kappa sequence, as numbered by the EU index.

Table 1 below provides exemplary CH1/CL orthogonal modifications comprising an engineered disulfide bridge between CH1 and CL, numbered according to the EU index.

TABLE 1
Exemplary CH1/CL engineered
disulfide bridges

| CH1 mutation | CL mutation |
|---|---|
| A141C | F118C |
| H168C | T164C |
| L128C | F118C |
| P171C | S162C |

In a series of preferred embodiments, the mutations that provide non-endogenous (engineered) cysteine amino acids are a F118C mutation in the CL sequence with a corresponding A141C in the CH1 sequence, or a F118C mutation in the CL sequence with a corresponding L128C in the CH1 sequence, a T164C mutation in the CL sequence with a corresponding H168C mutation in the CH1 sequence, or a S162C mutation in the CL sequence with a corresponding P171C mutation in the CH1 sequence, as numbered by the Eu index.

### 6.3.2.1.2 CH1/CL Orthogonal Modifications: Engineered Charge-Pair Mutations

In a variety of embodiments, the orthogonal modifications in the CL sequence and the CH1 sequence are charge-pair mutations.

In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches.

In some cases, the CH1/CL charge-pair mutations are a N138K mutation in the CL sequence with a corresponding G166D in the CH1 sequence, or a N138D mutation in the CL sequence with a corresponding G166K in the CH1 sequence, as numbered by the Eu index. In some embodiments, the charge-pair mutations are a P127E mutation in CH1 sequence with a corresponding E123K mutation in the corresponding CL sequence. In some embodiments, the charge-pair mutations are a P127K mutation in CH1 sequence with a corresponding E123 (not mutated) in the corresponding CL sequence.

Table 2 below provides exemplary CH1/CL orthogonal charged-pair modifications.

TABLE 2
exemplary CH1/CL orthogonal
charged-pair modifications

| CH1 mutation | CL mutation |
|---|---|
| G166K | N138K |
| G166K | N138D |
| P127E | E123K |
| P127E | No mutation (E123) |
| P127K | E123K |
| P127K | No mutation (E123) |

### 6.3.2.1.3 Combinations of CH1/CL Orthogonal Modifications

In certain embodiments, the CH1 and C_{L} domains of a single CH1/CL pair separately contain two or more respectively orthogonal modifications in endogenous CH1 and CL sequences. For instance, the CH1 and CL sequence may contain a first orthogonal modification and a second orthogonal modification in the endogenous CH1 and CL sequences. The two or more respectively orthogonal modifications in endogenous CH1 and CL sequences can be selected from any of the CH1/CL orthogonal modifications described herein.

In some embodiments, the first orthogonal modification is an orthogonal charge-pair mutation, and the second orthogonal modification is an orthogonal engineered disulfide bridge. In some embodiments, the first orthogonal modification is an orthogonal charge-pair mutation as described in Table 2, and the additional orthogonal modification comprise an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety.

In some embodiments, the first orthogonal modification is an orthogonal charge-pair mutation as described in Table 2, and the additional orthogonal modification comprise an engineered disulfide bridge as described in Table 1. In some embodiments, the first orthogonal modification comprises an L128C mutation in the CH1 sequence and an F118C mutation in the CL sequence, and the second orthogonal modification comprises a modification of residue 166 in the same CH1 sequence and a modification of residue 138 in the same CL sequence.

In some embodiments, the first orthogonal modification comprises an L128C mutation in the CH1 sequence and an F118C mutation in the CL sequence, and the second orthogonal modification comprises a G166D mutation in the CH1 sequence and a N138K mutation in the CL sequence. In some embodiments, the first orthogonal modification comprises an L128C mutation in the CH1 sequence and an F118C mutation in the CL sequence, and the second orthogonal modification comprises a G166K mutation in the CH1 sequence and a N138D mutation in the CL sequence.

### 6.3.3. CH2 Domains

In various embodiments, at least one of the first and second polypeptide chains of the antibody construct further comprises a CH2 domain. In various embodiments, both the first polypeptide and the second polypeptide comprises a CH2 domain.

In some embodiments, the antibody construct has more than one paired set of CH2 domains. In various of these embodiments, a first set of paired CH2 domains has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype but not significantly interact with the CH2 amino acid sequences from another isotype present in the antibody construct.

In particular embodiments, the first set of CH2 amino acid sequences is from the same isotype as the other non-CH2 domains in the antibody construct. In a specific embodiment, the first set has CH2 amino acid sequences from an IgG isotype and the one or more orthologous sets have CH2 amino acid sequences from an IgM or IgE isotype.

In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the antibody constructs described herein are described in more detail in international PCT applications WO2017/011342 and WO2017/106462, herein incorporated by reference in their entireties.

In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species.

### 6.3.4. CH3 Domains

The CH3 domains of the antibody constructs described herein have amino acid sequences derived from domains that are naturally positioned at the C-terminus of an antibody heavy chain, into which the mutations described above are engineered.

In a variety of embodiments, the CH3 sequences are mammalian sequences, including, but not limited to, mouse, rat, hamster, rabbit, canine, feline, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype. In specific embodiments, the CH3 sequences are from an IgG isotype. In a preferred embodiment, the CH3 sequences are from an IgG1 isotype. In some embodiments, the CH3 sequence is from an IgA isotype.

In certain embodiments, the CH3 sequences are CH4 sequences from an IgE or IgM isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330.

In certain embodiments, the CH3 sequences are engineered to reduce immunogenicity by replacing specific amino acids of one allotype with those of another allotype (referred to herein as isoallotype mutations), as described in more detail in Stickler et al. (Genes Immun. 2011 April; 12(3): 213-221), which is herein incorporated by reference for all that it teaches. In particular embodiments, specific amino acids of the Glml allotype are replaced. In a preferred embodiment, isoallotype mutations D356E and L358M are made in the CH3 sequence.

In some embodiments, there are no additional engineered mutations in the first or second CH3 domains. In some embodiments, the CH3 sequences are endogenous sequences that have one or more engineered mutations additional to those described above, as described below.

### 6.3.5. Additional Engineered Orthogonal Mutations in the CH3 Domain

In some embodiments, there is at least one additional orthogonal mutation engineered into the first and/or second CH3 domain.

### 6.3.5.1 Knob-in-Hole

In various embodiments, the first and second CH3 domains further comprise orthogonal knob-in-hole (“K-I-H”) mutations.

As used herein, knob-in-hole mutations are mutations that change the steric features of a first domain's surface such that the first domain will preferentially associate with a second domain having complementary steric mutations relative to association with domains without the complementary steric mutations. Knob-in-hole mutations are described in greater detail in U.S. Pat. Nos. 5,821,333 and 8,216,805, each of which is incorporated herein in its entirety.

In some embodiments, the at least one additional engineered mutation is a knob mutation in the first CH3 domain and a hole mutation in the second CH3 domain. In some embodiments, there is a hole mutation in the first CH3 domain and a knob mutation in the second CH3 domain.

In certain embodiments, the knob mutation is T366W or T366Y.

In certain embodiments, the hole mutation is selected from T366S, L368A, F405T, Y407V, or Y407T. In a specific embodiment, the hole mutation is F405T.

In certain embodiments, the knob-in-hole mutations are a T366W mutation in the first CH3 domain and a Y407A mutation in the second CH3 domain. In certain embodiments, the knob-in-hole mutations are a T366W mutation in the second CH3 domain and a Y407A mutation in the first CH3 domain.

In certain embodiments, the knob-in-hole mutations are a T366Y mutation in the first CH3 domain and a Y407T mutation in the second domain. In certain embodiments, the knob-in-hole mutations are a T366Y mutation in the second CH3 domain, and a Y407T mutation in the first domain.

In certain embodiments, the knob-in-hole mutations are a T394W in the first CH3 domain, and F405A in the second CH3 domain. In certain embodiments, the knob-in-hole mutations are a T394W in the second CH3 domain, and F405A in the first CH3 domain.

In certain embodiments, the knob-in-hole mutations are a T366Y mutation and a F405A in the first CH3 domain and a T394W mutation and a Y407T mutation in the second CH3 domain. In certain embodiments, the knob-in-hole mutations are a T366Y mutation and a F405A in the second CH3 domain and a T394W mutation and a Y407T mutation in the first CH3 domain.

### 6.3.5.2 Engineered Charge Pair

In various embodiments, the first and second CH3 domains further comprise orthogonal charge-pair mutations.

As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562; 9,248,182; and 9,358,286, each of which is incorporated herein by reference herein in its entirety.

In certain embodiments, the charge-pair mutations are a T366K mutation in the first CH3 domain and a L351D mutation in the second CH3 domain. In certain embodiments, the charge-pair mutations are a T366K mutation in the second CH3 domain and a L351D mutation in the first CH3 domain.

### 6.3.6. Specific Bivalent Antibody Constructs

In various embodiments, the antibody construct has the architecture shown in FIG. 51.

6.3.6.1 Domain A

With reference to FIG. 51, in various embodiments of the antibody constructs described herein, domain A has a variable region domain amino acid sequence. In a preferred embodiment, domain A has a VL antibody domain sequence and domain F has a VH antibody domain sequence. In some embodiments, domain A has a VH antibody domain sequence and domain F has a VL antibody domain sequence.

In the antibody constructs described herein, the C-terminus of domain A is connected to the N-terminus of domain B. In certain embodiments, domain A has a VL amino acid sequence that is mutated at its C-terminus at the junction between domain A and domain B.

### 6.3.6.2 Domain B

With reference to FIG. 51, in various embodiments of the antibody constructs described herein, domain B has a constant region domain sequence.

In some embodiments, domain B has a CH3 sequence.

In certain embodiments, domain B has a CH3 sequence comprising “knob-in-hole” (“KIH”) orthogonal mutations.

In certain embodiments, domain B has a CH3 sequence and either a S354C or a Y349C mutation that forms an engineered disulfide bridge with a CH3 domain containing an orthogonal mutation.

In certain embodiments, domain B has first CH3 domain sequence, wherein Y349 of the first CH3 domain is substituted with cysteine (C) (Y349C), wherein the positions are numbered according to the Eu index.

In certain embodiments, domain B has a second CH3 domain sequence, wherein 5354 of the second CH3 domain is substituted with cysteine (C) (S354C), and E357 of the second CH3 domain is substituted with a hydrophobic or aromatic amino acid, wherein the positions are numbered according to the Eu index. In particular embodiments, the second CH3 domain has an E357W mutation.

In certain embodiments, domain B has a human IgG1 CH3 amino acid sequence with the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 44W, 446G, 447K.

In certain embodiments, domain B has a human IgG1 CH3 sequence with a K447C mutation incorporated into an otherwise endogenous CH3 sequence.

In some embodiments, the constant region sequence is an orthologous CH2 sequence. In some embodiments, domain B has a CH2 sequence from IgE. In some embodiments, domain B has a CH2 sequence from IgM.

In some embodiments, for example wherein the valency of the binding molecule is three or greater than three, the constant region sequence is a CH1 or CL sequence. In some embodiments, domain B has a CH1 sequence. In some embodiments, the constant region sequence is a CL sequence. In some embodiments, the CH1 or CL sequence comprises one or more CH1 or CL orthogonal modifications described herein.

In the antibody constructs described herein, the N-terminus of domain B is connected to the C-terminus of domain A. In certain embodiments, domain B has a CH3 amino acid sequence that is mutated at its N-terminus at the junction between domain A and domain B.

In the antibody constructs described herein, the C-terminus of domain B is connected to the N-terminus of domain D. In certain embodiments, domain B has a CH3 amino acid sequence that is extended at the C-terminus at the junction between domain B and domain D.

In some embodiments, domain B comprises a human IgA CH3 sequence. In some embodiments, the IgA CH3 sequence comprises a CH3 linker sequence.

### 6.3.6.3 Domain D

With reference to FIG. 51, in various embodiments of the antibody constructs described herein, domain D has a constant region amino acid sequence.

In a preferred series of embodiments, domain D has a CH2 amino acid sequence. In certain embodiments, the CH2 sequences are endogenous sequences. In particular embodiments, the sequence is UniProt accession number P01857 amino acids 111-223. In a preferred embodiment, the CH2 sequences have an N-terminal hinge region peptide that connects the N-terminal variable domain-constant domain segment to the CH2 domain. In some embodiments, the CH2 sequence comprises one or more mutations that modulate effector function. In certain embodiments, the CH2 sequence comprises one or more mutations that reduce effector function. In some embodiments, the CH2 sequence comprises one or more mutations that modulate FcRN binding. In certain embodiments, the CH2 sequence comprises one or more mutations that reduce FcRN binding.

In the antibody constructs described herein, the N-terminus of domain D is connected to the C-terminus of domain B. In certain embodiments, domain B has a CH3 amino acid sequence that is extended at the C-terminus at the junction between domain D and domain B.

### 6.3.6.4 Domain E

With reference to FIG. 51, in various embodiments of the antibody constructs described herein, domain E has a constant region domain amino acid sequence.

In certain embodiments, the constant region sequence is a CH3 sequence.

In certain embodiments, domain E has a CH3 sequence comprising “knob-in-hole” (“KIH”) orthogonal mutations.

In certain embodiments, domain E has a CH3 sequence and either a S354C or a Y349C mutation that forms an engineered disulfide bridge with a CH3 domain containing an orthogonal mutation.

In certain embodiments, domain E has first CH3 domain sequence, wherein Y349 of the first CH3 domain is substituted with cysteine (C) (Y349C).

In certain embodiments, domain E has a second CH3 domain sequence, wherein S354 of the second CH3 domain is substituted with cysteine (C) (S354C), and E357 of the second CH3 domain is substituted with a hydrophobic or aromatic amino acid, wherein the positions are numbered according to the Eu index. In particular embodiments, the second CH3 domain has an E357W mutation.

In certain embodiments, the constant region domain sequence is a CH1 sequence. In particular embodiments, the CH1 amino acid sequence of domain E is the only CH1 amino acid sequence in the binding molecule. In certain embodiments, the N-terminus of the CH1 domain is connected to the C-terminus of a CH2 domain.

In certain embodiments, the constant region sequence is a CL sequence. In certain embodiments, the N-terminus of the C_{L} domain is connected to the C-terminus of a CH2 domain.

### 6.3.6.5 Domain F

With reference to FIG. 51, in various embodiments of the antibody constructs described herein, domain F has a variable region domain amino acid sequence. In a preferred embodiment, domain F has a VH antibody domain sequence. In some embodiments, domain F has a VL antibody domain sequence.

### 6.3.6.6 Domain G

With reference to FIG. 51, in various embodiments of the antibody constructs described herein, domain G has a constant region amino acid sequence.

In some embodiments, domain G has a CH3 amino acid sequence.

In certain embodiments, domain G has a human IgG1 CH3 sequence with the following mutational changes: S354C; and a tripeptide insertion, 445P, 446G, 447K. In some embodiments, domain G has a human IgG1 CH3 sequence with the following mutational changes: S354C; and 445P, 446G, 447K tripeptide insertion. In some preferred embodiments, domain G has a human IgG1 CH3 sequence with the following changes: L351D, and a tripeptide insertion of 445G, 446E, 447C.

In certain embodiments, domain G has a CH3 sequence comprising “knob-in-hole” (“KIH”) orthogonal mutations.

In certain embodiments, domain G has a CH3 sequence and either a S354C or a Y349C mutation that forms an engineered disulfide bridge with a CH3 domain containing an orthogonal mutation.

In some embodiments, domain G has a human IgA CH3 sequence.

In some embodiments, domain G has a CL sequence.

In some embodiments, domain G has a CH2 sequence from IgE. In some embodiments, domain G has a CH2 sequence from IgM.

In particular embodiments, for example wherein the valency of the binding molecule is three or greater than three, the constant region sequence is a CH1 or CL sequence. In some embodiments wherein domain B is a CL sequence, domain G is a CH1 sequence. In some embodiments, the CH1 or CL sequence comprises one or more CH1 or Cl orthogonal modifications described herein.

In some embodiments, the C-terminus of domain G is connected to the N-terminus of domain D. In certain embodiments, domain G has a CH3 amino acid sequence that is extended at the C-terminus at the junction between domain G and domain D.

### 6.3.6.7 Domain H

With reference to FIG. 51, in various embodiments of the antibody constructs described herein, domain H has a variable region domain amino acid sequence. In a preferred embodiment, domain H has a VL antibody domain sequence. In some embodiments, domain H has a VH antibody domain sequence.

### 6.3.6.8 Domain I

With reference to FIG. 51, in various embodiments of the antibody constructs described herein, domain I has a constant region domain amino acid sequence.

In a series of preferred embodiments of the binding molecules, domain I has a CL amino acid sequence. In another series of embodiments, domain I has a CH1 amino acid sequence.

### 6.3.6.9 Domain J

With reference to FIG. 51, in various embodiments of the antibody constructs described herein, domain J has a CH2 amino acid sequence. In a preferred embodiment, the CH2 amino acid sequence has an N-terminal hinge region that connects domain J to domain I. In some embodiments, the CH2 sequence comprises one or more mutations that modulate effector function. In certain embodiments, the CH2 sequence comprises one or more mutations that reduce effector function. In some embodiments, the CH2 sequence comprises one or more mutations that modulate FcRN binding. In certain embodiments, the CH2 sequence comprises one or more mutations that reduce FcRN binding.

The C-terminus of domain J is connected to the N-terminus of domain K. In particular embodiments, domain J is connected to the N-terminus of domain K that has a CH1 amino acid sequence or CL amino acid sequence.

### 6.3.6.10 Domain K

With reference to FIG. 51, in various embodiments of the antibody constructs described herein, domain K has a constant region domain amino acid sequence.

In some embodiments, domain K has a CH3 sequence.

In certain embodiments, domain K has a CH3 sequence comprising “knob-in-hole” (“KIH”) orthogonal mutations.

In certain embodiments, domain K has a CH3 sequence and either a S354C or a Y349C mutation that forms an engineered disulfide bridge with a CH3 domain containing an orthogonal mutation.

In certain embodiments, domain K has first CH3 domain sequence, wherein Y349 of the first CH3 domain is substituted with cysteine (C) (Y349C).

In certain embodiments, domain K has a second CH3 domain sequence, wherein S354 of the second CH3 domain is substituted with cysteine (C) (S354C), and E357 of the second CH3 domain is substituted with a hydrophobic or aromatic amino acid, wherein the positions are numbered according to the Eu index. In particular embodiments, the second CH3 domain has an E357W mutation.

In some embodiments, knob-in-hole orthogonal mutations are combined with isoallotype mutations. In certain embodiments, the knob mutation is T366W or T366Y. In certain embodiments, the hole mutation is selected from T366S, L368A, F405T, Y407V, or Y407T. In a specific embodiment, the hole mutation is F405T.

In certain embodiments, the constant region domain sequence is a CH1 sequence. In particular embodiments, the CH1 amino acid sequence of domain K is the only CH1 amino acid sequence in the binding molecule. In certain embodiments, the N-terminus of the CH1 domain is connected to the C-terminus of a CH2 domain. In certain embodiments, the constant region sequence is a CL sequence. In certain embodiments, the N-terminus of the C_{L} domain is connected to the C-terminus of a CH2 domain.

### 6.3.6.11 Domain L

With reference to FIG. 51, in various embodiments of the antibody constructs described herein, domain L has a variable region domain amino acid sequence. In a preferred embodiment, domain L has a VH antibody domain sequence. In some embodiments, domain L has a VL antibody domain sequence.

### 6.3.6.12 Domain M

With reference to FIG. 51, in various embodiments of the antibody constructs described herein, domain M has a constant region domain amino acid sequence. In a series of preferred embodiments, domain I has a CH1 amino acid sequence and domain M has a CL amino acid sequence. In another series of preferred embodiments, domain I has a CL amino acid sequence and domain M has a CH1 amino acid sequence.

### 6.3.6.13 Pairing of Domains A & F

In the antibody constructs illustrated in FIG. 51, a domain “A” VL or VH amino acid sequence and a cognate domain “F” VH or VL amino acid sequence are associated and form an antigen binding site (ABS). The A:F antigen binding site (ABS) is capable of specifically binding an epitope of an antigen.

In a variety of multivalent embodiments, the ABS formed by domains A and F (A:F) is identical in sequence to one or more other ABSs within the binding molecule and therefore has the same recognition specificity as the one or more other sequence-identical ABSs within the binding molecule.

In a variety of multivalent embodiments, the A:F ABS is non-identical in sequence to one or more other ABSs within the binding molecule. In certain embodiments, the A:F ABS has a recognition specificity different from that of one or more other sequence-non-identical ABSs in the binding molecule. In particular embodiments, the A:F ABS recognizes a different antigen from that recognized by at least one other sequence-non-identical ABS in the binding molecule. In particular embodiments, the A:F ABS recognizes a different epitope of an antigen that is also recognized by at least one other sequence-non-identical ABS in the binding molecule. In these embodiments, the ABS formed by domains A and F recognizes an epitope of antigen, wherein one or more other ABSs within the binding molecule recognizes the same antigen but not the same epitope.

### 6.3.6.14 Pairing of Domains B & G

In the antibody constructs illustrated in FIG. 51, a domain “B” constant region amino acid sequence and a domain “G” constant region amino acid sequence are associated.

In some embodiments, domain B and domain G have CH3 amino acid sequences.

In certain embodiments, domain B is a first CH3 domain and domain G is a second CH3 domain, wherein Y349 of the first CH3 domain is substituted with cysteine (C) (Y349C), 5354 of the second CH3 domain is substituted with cysteine (C) (S354C), and E357 of the second CH3 domain is substituted with a hydrophobic or aromatic amino acid, wherein the positions are numbered according to the Eu index.

In certain embodiments, domain B is a second CH3 domain and domain G is a first CH3 domain, wherein Y349 of the first CH3 domain is substituted with cysteine (C) (Y349C), S354 of the second CH3 domain is substituted with cysteine (C) (S354C), and E357 of the second CH3 domain is substituted with a hydrophobic or aromatic amino acid, wherein the positions are numbered according to the Eu index.

In various embodiments, the amino acid sequences of the B and the G domains are identical. In certain of these embodiments, the sequence is an endogenous CH3 sequence. The sequence may be a CH3 sequence from human IgG1. The sequence may be a sequence from human IgA.

In a variety of embodiments, the amino acid sequences of the B and the G domains are different, and separately comprise respectively orthogonal modifications in an endogenous CH3 sequence, wherein the B domain interacts with the G domain, and wherein neither the B domain nor the G domain significantly interacts with a CH3 domain lacking the orthogonal modification.

In particular embodiments, it is desirable to reduce an undesired association of domains B or G containing CH3 sequences with domains E and K also containing CH3 sequences. In such cases, use of CH3 sequences from human IgA (IgA-CH3) in domains B and/or G may improve antibody assembly and stability by reducing such undesired associations. In some embodiments of a binding molecule wherein domains E and K comprise IgG-CH3 sequences, domains B and G comprise IgA-CH3 sequences.

### 6.3.6.15 Pairing of Domains E & K

In certain embodiments, domain E is a first CH3 domain and domain K is a second CH3 domain, wherein Y349 of the first CH3 domain is substituted with cysteine (C) (Y349C), 5354 of the second CH3 domain is substituted with cysteine (C) (S354C), and E357 of the second CH3 domain is substituted with a hydrophobic or aromatic amino acid, wherein the positions are numbered according to the Eu index.

In certain embodiments, domain E is a second CH3 domain and domain K is a first CH3 domain, wherein Y349 of the first CH3 domain is substituted with cysteine (C) (Y349C), S354 of the second CH3 domain is substituted with cysteine (C) (S354C), and E357 of the second CH3 domain is substituted with a hydrophobic or aromatic amino acid, wherein the positions are numbered according to the Eu index.

In certain embodiments, the different sequences separately comprise respectively orthogonal modifications in an endogenous CH3 sequence, wherein the E domain interacts with the K domain, and wherein neither the E domain nor the K domain significantly interacts with a CH3 domain lacking the orthogonal modification. In certain embodiments, the orthogonal modifications include, but are not limited to, engineered disulfide bridges, knob-in-hole mutations, and charge-pair mutations. In particular embodiments, orthogonal modifications include a combination of orthogonal modifications selected from, but not limited to, engineered disulfide bridges, knob-in-hole mutations, and charge-pair mutations.

In particular embodiments, the orthogonal modifications can be combined with amino acid substitutions that reduce immunogenicity, such as isoallotype mutations.

In various embodiments, the amino acid sequences of the E and the K domains are identical.

### 6.3.6.16 Pairing of Domains H & L

In a variety of embodiments, domain H has a VL sequence and domain L has a VH sequence and domain “H” VL a domain “L” VH amino acid sequence are associated and form an antigen binding site (ABS). The H:L antigen binding site (ABS) is capable of specifically binding an epitope of an antigen.

In preferred embodiments, domain H has a VL amino acid sequence, domain I has a CL amino acid sequence, domain L has a VH amino acid sequence, and domain M has a CH1 amino acid sequence.

In a variety of embodiments, the amino acid sequences of the H domain and the L domain separately comprise respectively orthogonal modifications in an endogenous sequence, wherein the H domain interacts with the L domain, and wherein neither the H domain nor the L domain significantly interacts with a domain lacking the orthogonal modification. In a series of embodiments, the orthogonal mutations in the H domain are in a VL sequence and the orthogonal mutations in the L domain are in VH sequence. In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference in its entirety.

In certain embodiments, the interaction between the A domain and the F domain form a first antigen binding site specific for a first antigen, and the interaction between the H domain and the L domain form a second antigen binding site specific for a second antigen. In certain embodiments, the interaction between the A domain and the F domain form a first antigen binding site specific for a first antigen, and the interaction between the H domain and the L domain form a second antigen binding site specific for the first antigen.

### 6.3.6.17 Pairing of domains I & M

In a variety of embodiments, domain I has a CL sequence and domain M has a CH1 sequence.

In a variety of embodiments, the amino acid sequences of the I domain and the M domain separately comprise respectively orthogonal modifications in an endogenous sequence, wherein the I domain interacts with the M domain, and wherein neither the I domain nor the M domain significantly interacts with a domain lacking the orthogonal modification. In a series of embodiments, the orthogonal mutations in the I domain are in a CL sequence and the orthogonal mutations in the M domain are in CH1 sequence.

### 6.3.6.18 Domain Junctions

### 6.3.6.18.1 Junctions Connecting VL and CH3 Domains

In a variety of embodiments, the amino acid sequence that forms a junction between the C-terminus of a V_{L} domain and the N-terminus of a CH3 domain is an engineered sequence.

In certain embodiments, one or more amino acids are deleted or added in the C-terminus of the V_{L} domain. In particular embodiments, A111 is deleted in the C-terminus of the V_{L} domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In certain embodiments, one or more amino acids are deleted or added to both the C-terminus of the V_{L} domain and the N-terminus of the CH3 domain. In particular embodiments, A111 is deleted in the C-terminus of the V_{L} domain and P343 is deleted in the N-terminus of the CH3 domain. In a preferred embodiment, A111 and V110 are deleted in the C-terminus of the V_{L} domain. In another preferred embodiment, A111 and V110 are deleted in the C-terminus of the V_{L} domain and the N-terminus of the CH3 domain has a P343V mutation.

### 6.3.6.18.2 Junctions Connecting VII and CH3 Domains

In a variety of embodiments, the amino acid sequence that forms a junction between the C-terminus of a V_{H} domain and the N-terminus of a CH3 domain is an engineered sequence.

In certain embodiments, one or more amino acids are deleted or added in the C-terminus of the V_{H} domain. In particular embodiments, K117 and G118 are deleted in the C-terminus of the V_{H} domain. In certain embodiments, one or more amino acids are deleted or added in the N-terminus of the CH3 domain. In particular embodiments, P343 is deleted in the N-terminus of the CH3 domain. In particular embodiments, P343 and R344 are deleted in the N-terminus of the CH3 domain. In particular embodiments, P343, R344, and E345 are deleted in the N-terminus of the CH3 domain. In certain embodiments, one or more amino acids are deleted or added to both the C-terminus of the V_{H} domain and the N-terminus of the CH3 domain. In a preferred embodiment, T116, K117, and G118 are deleted in the C-terminus of the V_{H} domain.

### 6.3.6.18.3 Junctions Connecting CH3 C-Terminus to CH2 N-Terminus (Hinge)

In some embodiments of the antibody constructs described herein, the N-terminus of the CH2 domain has a “hinge” region amino acid sequence. As used herein, hinge regions are sequences of an antibody heavy chain that link the N-terminal variable domain-constant domain segment of an antibody and a CH2 domain of an antibody. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains).

In a variety of embodiments, a CH3 amino acid sequence is extended at the C-terminus at the junction between the C-terminus of the CH3 domain and the N-terminus of a CH2 domain. In certain embodiments, a CH3 amino acid sequence is extended at the C-terminus at the junction between the C-terminus of the CH3 domain and a hinge region, which in turn is connected to the N-terminus of a CH2 domain. In a preferred embodiment, the CH3 amino acid sequence is extended by inserting a CH3 amino acid extension sequence (“CH3 linker sequence” or “CH3 linker”). In some embodiments, the CH3 amino acid extension sequence is followed by the DKTHT motif of an IgG1 hinge region. In some embodiments, the CH3 amino acid extension sequence is 3-10 amino acids in length. In some embodiments, the CH3 amino acid extension sequence is 3-8 amino acids in length. In some embodiments, the CH3 amino acid extension sequence is 3-6 amino acids in length.

In some embodiments, the CH3 amino acid extension sequence is a PGK tripeptide. In some embodiments, the CH3 amino acid extension sequence is an AGC tripeptide. In some embodiments, the CH3 amino acid extension sequence is a GEC tripeptide. In some embodiments, the CH3 amino acid extension sequence is AGKC. In some embodiments, the CH3 amino acid extension sequence is PGKC. In some embodiments, the CH3 amino acid extension sequence is AGKGC. In some embodiments, the CH3 amino acid extension sequence is AGKGSC.

In a particular embodiment, the extension at the C-terminus of the CH3 domain incorporates amino acid sequences that can form a disulfide bond with orthogonal C-terminal extension of another CH3 domain. In a preferred embodiment, the extension at the C-terminus of the CH3 domain incorporates a KSC tripeptide sequence that is followed by the DKTHT motif of an IgG1 hinge region that forms a disulfide bond with orthogonal C-terminal extension of another CH3 domain that incorporates a GEC motif of a kappa light chain.

In some embodiments of a binding molecule wherein domains B and G comprise CH3 amino acid sequences, domain B comprises a first CH3 linker sequence and domain G comprises a second CH3 linker sequence. In some embodiments, the first CH3 linker sequence associates with the second CH3 linker sequence by formation of a disulfide bridge between cysteine residues of the first and second CH3 linker sequences. In some embodiments, the first CH3 linker and the second CH3 linker are identical. In some embodiments, the first CH3 linker and second CH3 linker are non-identical. In some embodiments, the first CH3 linker and second CH3 linker differ in length by 1-6 amino acids. In some embodiments, the first CH3 linker and second CH3 linker differ in length by 1-3 amino acids.

In preferred embodiments, the first CH3 linker is AGC and the second CH3 linker is AGKGSC. In some embodiments, the first CH3 linker is AGKGC and the second CH3 linker is AGC. In some embodiments, the first CH3 linker is AGKGSC and the second CH3 linker is AGC. In some embodiments, the first CH3 linker is AGKC and the second CH3 linker is AGC.

### 6.3.6.18.4 Junctions Connecting CL C-Terminus and CH2 N-Terminus (Hinge)

In a variety of embodiments, a CL amino acid sequence is connected through its C-terminus to a hinge region, which in turn is connected to the N-terminus of a CH2 domain.

### 6.3.6.18.5 Junctions Connecting CH2 C-Terminus to Constant Region Domain

In a variety of embodiments, a CH2 amino acid sequence is connected through its C-terminus to the N-terminus of a constant region domain. In a preferred embodiment, the CH2 sequence is connected to a CH3 sequence via its endogenous sequence. In other embodiments, the CH2 sequence is connected to a CH1 or CL sequence. Examples discussing connecting a CH2 sequence to a CH1 or CL sequence are described in more detail in U.S. Pat. No. 8,242,247, which is hereby incorporated by reference in its entirety.

### 6.3.6.19 Bivalent Bispecific B-Body “BC1”

With reference to FIG. 51, in a series of embodiments, the antibody construct has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a T366K mutation and a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a L351D mutation and a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

The orthogonal mutations described herein can be further engineered into the E:K paired domains of this construct.

### 6.3.6.20 Bivalent Bispecific B-Body “BC6”

With reference to FIG. 51, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a C-terminal extension incorporating a KSC tripeptide sequence that is followed by the DKTHT motif of an IgG1 hinge region, domain D has a human IgG1 CH2 amino acid sequence, and domain E has human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a C-terminal extension incorporating a GEC amino acid disulfide motif; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V mutation; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

The orthogonal mutations described herein can be further engineered into the E:K paired domains.

### 6.3.6.21 Bivalent Bispecific B-Body “BC28”

With reference to FIG. 51, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation and a C-terminal extension incorporating a PGK tripeptide sequence that is followed by the DKTHT motif of an IgG1 hinge region, domain D has a human IgG1 CH2 amino acid sequence, and domain E has a human IgG1 CH3 amino acid with a S354C and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-teuninus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, a D356E, a L358M, a T366S, a L368A, and a Y407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 CH1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

The orthogonal mutations described herein can be further engineered into the B:G paired domains or E:K paired domains.

### 6.3.6.22 Bivalent Bispecific B-Body “BC44”

With reference to FIG. 51, in a series of embodiments, the binding molecule has a first, second, third, and fourth polypeptide chain, wherein (a) the first polypeptide chain comprises a domain A, a domain B, a domain D, and a domain E, wherein the domains are arranged, from N-terminus to C-terminus, in a A-B-D-E orientation, and domain A has a first VL amino acid sequence, domain B has a human IgG1 CH3 amino acid sequence with a Y349C mutation, a P343V mutation, and a C-terminal extension incorporating a PGK tripeptide sequence that is followed by the DKTHT motif of an IgG1 hinge region, domain D has a human IgG1 CH2 amino acid sequence, and domain E has human IgG1 CH3 amino acid with a S354C mutation and a T366W mutation; (b) the second polypeptide chain has a domain F and a domain G, wherein the domains are arranged, from N-terminus to C-terminus, in a F-G orientation, and wherein domain F has a first VH amino acid sequence and domain G has a human IgG1 CH3 amino acid sequence with a S354C mutation and a C-terminal extension incorporating a PGK tripeptide sequence; (c) the third polypeptide chain has a domain H, a domain I, a domain J, and a domain K, wherein the domains are arranged, from N-terminus to C-terminus, in a H-I-J-K orientation, and wherein domain H has a second VL amino acid sequence, domain I has a human CL kappa amino acid sequence, domain J has a human IgG1 CH2 amino acid sequence, and K has a human IgG1 CH3 amino acid sequence with a Y349C, T366S, L368A, and aY407V; (d) the fourth polypeptide chain has a domain L and a domain M, wherein the domains are arranged, from N-terminus to C-terminus, in a L-M orientation, and wherein domain L has a second VH amino acid sequence and domain M has a human IgG1 amino acid sequence; (e) the first and the second polypeptides are associated through an interaction between the A and the F domains and an interaction between the B and the G domains; (f) the third and the fourth polypeptides are associated through an interaction between the H and the L domains and an interaction between the I and the M domains; and (g) the first and the third polypeptides are associated through an interaction between the D and the J domains and an interaction between the E and the K domains to form the binding molecule; (h) domain A and domain F form a first antigen binding site specific for a first antigen; and (i) domain H and domain L form a second antigen binding site specific for a second antigen.

The orthogonal mutations described herein can be further engineered into the B:G paired domains or E:K paired domains.

### 6.4. ANTIGEN SPECIFICITIES

### 6.4.1. B7H3

The antibody constructs disclosed herein have an antigen binding site (ABS) specific for B7 Homolog 3 (B7H3), also known as Cluster of Differentiation 276 (CD276).

In some embodiments, B7H3 is a cell surface antigen on target tumor cells. In some embodiments, the target tumor cells are neuroblastoma, melanoma, sarcoma, or small cell lung cancer.

In preferred embodiments, the antibody constructs disclosed herein comprise a first ABS that specifically binds to B7H3.

### 6.4.2. GD2

The antibody constructs disclosed herein have an antigen binding site (ABS) specific for disialoganglioside (GD2).

In some embodiments, GD2 is a cell surface antigen on target tumor cells. In some embodiments, the target tumor cells are neuroblastoma, melanoma, sarcoma, or small cell lung cancer.

In preferred embodiments, the antibody constructs disclosed herein comprise a second ABS that specifically binds to GD2.

### 6.5. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### 6.5.1. Example 1—INV721 Antibody

We constructed a new bivalent bispecific antibody, termed “INV721,” specific for a first tumor cell surface antigen, B7 Homolog 3 (B7H3), and a second tumor cell surface antigen, disialoganglioside (GD2). The antigen binding site (ABS) specific for B7H3 comprises the heavy chain variable region (VH) sequence of SEQ ID NO:2 and the light chain variable region sequence of SEQ ID NO:1. The antigen binding site (ABS) specific for GD2 comprises the VH sequence of SEQ ID NO:4 and the VL sequence of SEQ ID NO:3.

In greater detail, with domain and polypeptide chain references in accordance with FIG. 51, the architecture of INV721 is:
1^{st}polypeptide chain:
   Domain A=VL (B7H3 binder) (SEQ ID NO:1)
   Domain B=CH3
   Domain D=CH2
   Domain E=CH3
2^{nd} polypeptide chain:
   Domain F=VH (B7H3 binder) (SEQ ID NO:2)
   Domain G=CH3
3^{rd} polypeptide chain:
   Domain H=VL (GD2 binder) (SEQ ID NO:3)
   Domain I=CL (Kappa)
   Domain J=CH2
   Domain K=CH3
4^{th}polypeptide chain:
   Domain L=VH (GD2 binder) (SEQ ID NO:4)
   Domain M=CH1.

The A domain and F domain associate to form an antigen binding site (A:F) specific for B7H3. The H domain and the L domain associate to form an antigen binding site (H:L) specific for GD2.

With reference to FIG. 51, the B7H3 binding arm of INV721 comprises the first polypeptide chain and the second polypeptide chain. The B7H3 binding arm is I7-01.

With reference to FIG. 51, the GD2 binding arm of INV721 comprises the third polypeptide chain and the fourth polypeptide chain. In some preferred embodiments, the GD2 binding arm is GD2-5. In some preferred embodiments, the GD2 binding arm is GD2-7.

INV721 could readily be expressed at high levels using mammalian expression at concentrations greater than 100 μg/mL. We found that the bivalent bispecific INV721 protein could be purified using standard antibody purification methods.

### 6.5.2. Example 2—In Vitro Binding of INV721 in Cancer Cells

B78 melanoma tumor cells that express B7H3 and GD2 were incubated with (1) INV721, (2) an antibody construct comprising the ABS specific for B7H3 and a non-specific ABS, or (3) an antibody construct comprising the ABS specific for GD2 and a non-specific ABS. Following incubation, cells were washed and incubated with a secondary anti-human IgG antibody an analyzed by flow cytometry.

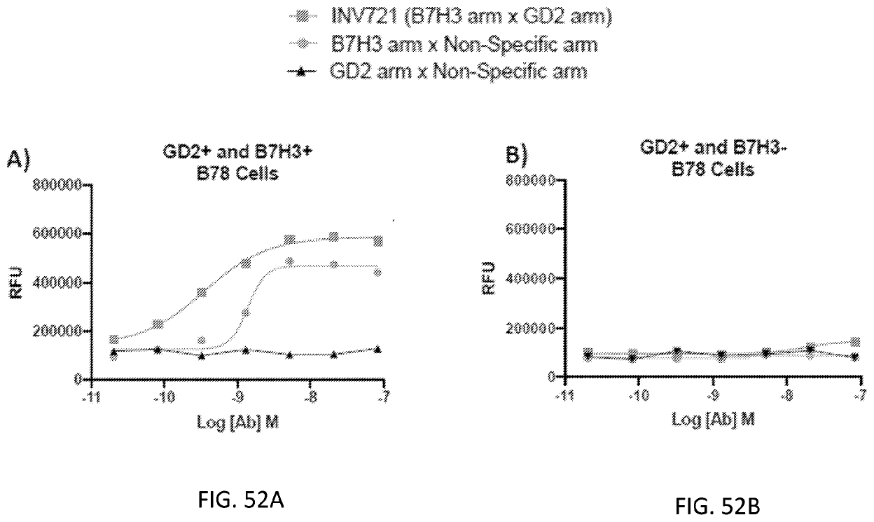

Results shown in FIG. 52A demonstrate that strong binding of INV721 to the B78 tumor cells was observed. Lower affinity binding for the B78 tumor cell was observed for the antibodies comprising only the ABS specific for B7H3 or GD2. By comparison, the monovalent anti-B7H3 antibody binds to B78 tumor cells with higher affinity than the monovalent anti-GD2 antibody but with lower affinity that INV721.

B78 melanoma tumor cells that express GD2 but do not express B7H3 were incubated with (1), (2), and (3), as described above. Results shown in FIG. 52B demonstrate that very little INV72 binds to tumor cells that express GD2 but do not express B7H3.

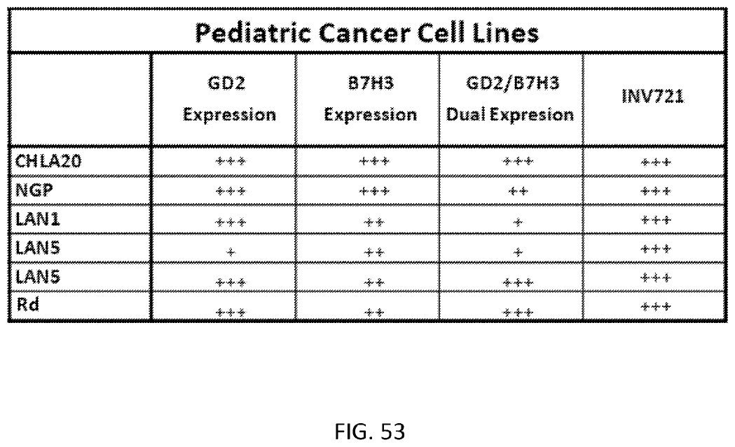

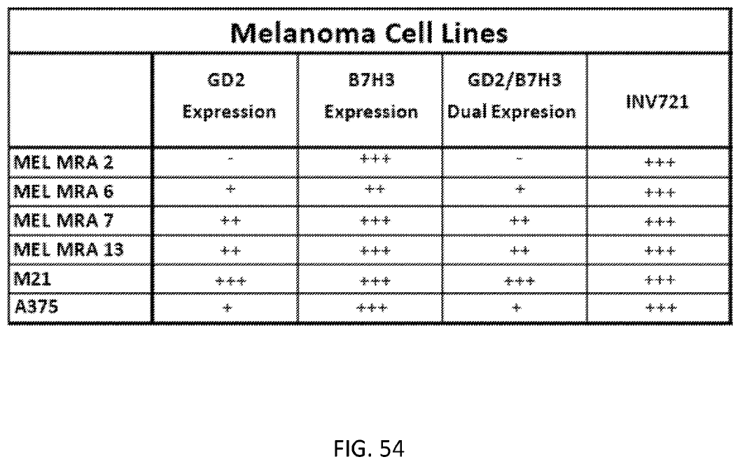

Other human melanoma and pediatric cancer cell lines, including cell lines originating from neuroblastoma and sarcoma tumors, were evaluated for GD2 and B7H3 expression and tested for INV721 binding. Results reported in FIG. 53 and FIG. 54 show that INV721 binds to all of the evaluated cancer cell lines that have high B7H3 expression, regardless of GD2 expression, when incubated with 1 μg of INV721 per 1 million cells.

### 6.5.3. Example 3—Internalization of INV721 Compared to Anti-B7H3 and Anti-GD2 Monoclonal Antibodies

The efficacy of therapeutic antibodies that mediate tumor cell death by antibody-dependent cell-mediated cytotoxicity (ADCC) and complement-dependent cytotoxicity (CDC) may be reduced by internalization of the antibodies into cells. Accordingly, we assessed cellular internalization of INV721 and the therapeutic anti-GD2 antibodies dinutuximab and hu14.18.

Internalization of INV721 was evaluated in various neuroblastoma cell lines that highly express GD2 and was compared to internalization of the anti-GD2 monoclonal antibodies dinutuximab, hu14.18, and GD2-5 (comprising the GD2 binding arm of INV721). Internalization of INV721 was also compared to the anti-B7H3 monoclonal antibody I7-01 (comprising the B7H3 binding arm of INV721) in LAN-1 cells

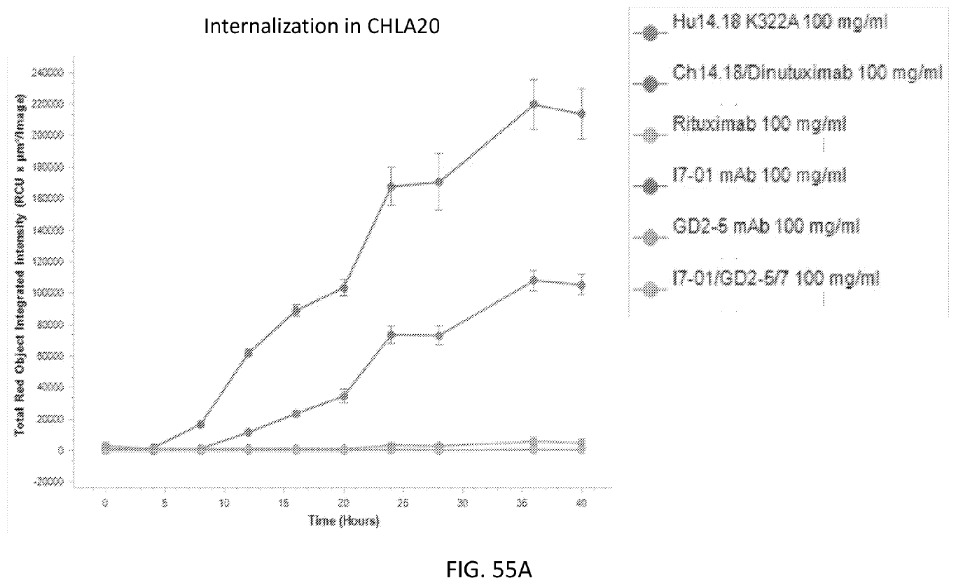

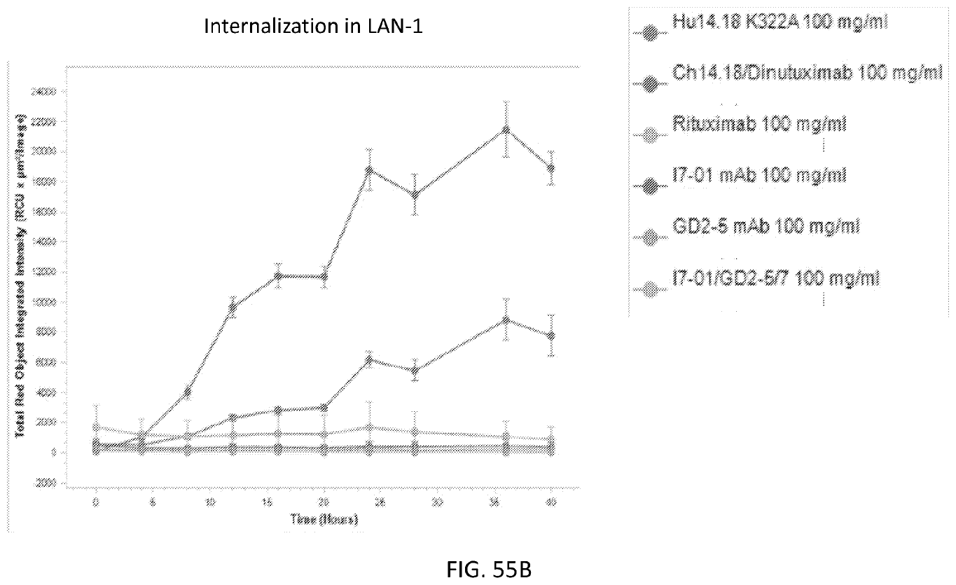

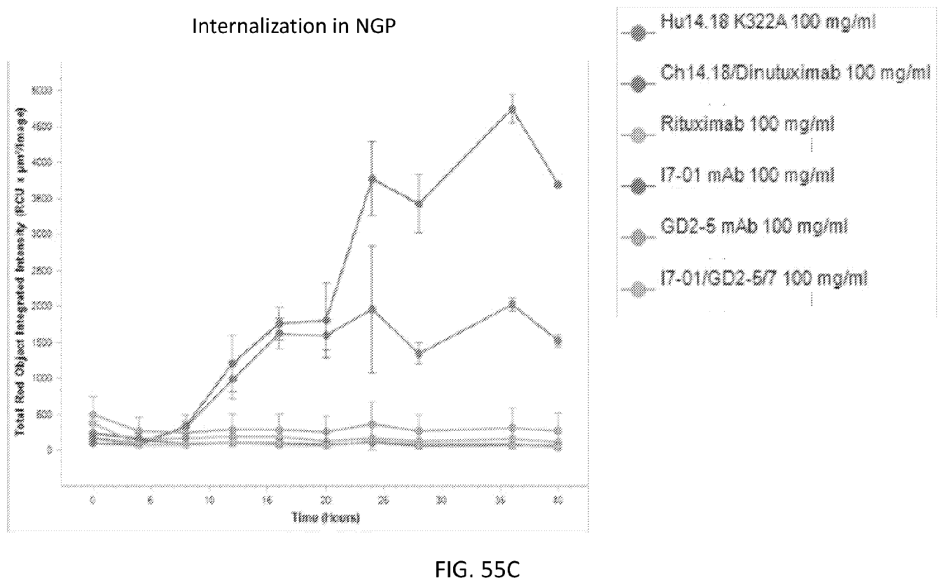

Results in FIGS. 55A-C show that internalization of INV721 (“I7-01/GD2-5/7”) is minimal compared to the anti-GD2 monoclonal antibodies dinutuximab and hu14.18 In addition, very little internalization of the anti-GD2 antibody GD2-5 was observed in CHLA20, LAN-1, and NGP neuroblastoma cell lines. No internalization of the anti-B7H3 antibody I7-01 was observed in LAN-1 cells which express both GD2 and B7H3.

### 6.5.4. Example 4—In Vitro Assessment of Tumor Growth Following INV721 Treatment

Antibody-dependent cell-mediated cytotoxicity (ADCC) assays were performed on B78 melanoma tumor cells in the presence or absence of INV721 in order to assess tumor growth following INV721 treatment. Briefly, B78 melanoma tumor cells expressing a nuclear localization sequence tagged with mKate2 and transduced with GD2 and/or B7H3 were plated in round bottom plates and allowed to form tumor spheroids. Healthy donor peripheral blood mononuclear cells (PBMCs) were added to the tumor spheroids with antibody (hu14.18, INV721, rituximab as a negative control, or media alone). Treated tumor spheroids were imaged using the IncuCyte cell imaging system. Staurosporine was included as a positive control. Tumor spheroids were incubated without PBMCs to test for direct antibody-mediated apoptosis.

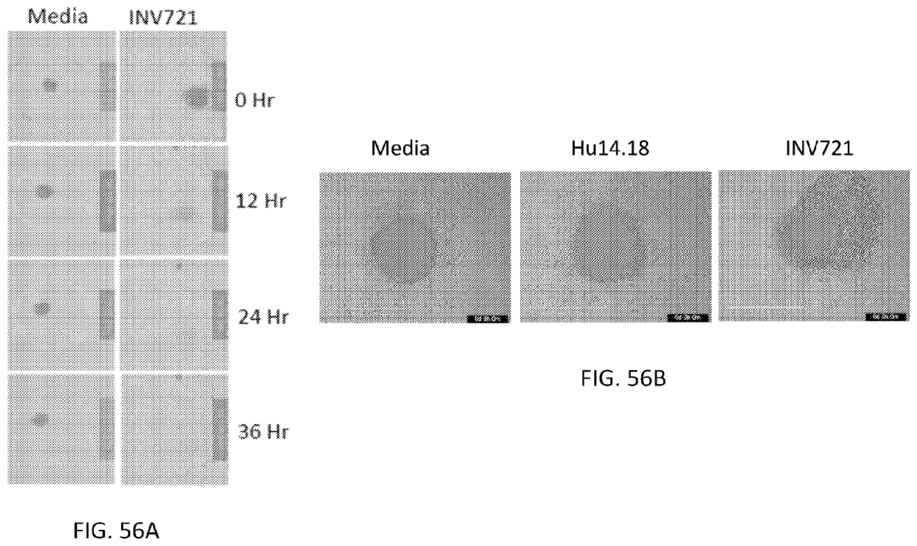

Results of the ADCC assays are shown in FIG. 56A and FIG. 56B. In FIG. X6A, dispersion of the bright compact signal following treatment with INV721 and PBMCs (right column) compared to PBMCs only (left column, “media”) demonstrate that INV721 inhibits tumor growth via ADCC. Results of ADCC assays in tumor spheroids treated with INV721 or the anti-GD2 monoclonal antibody Hu14.18 demonstrate greater efficacy of INV721 compared to hu14.18 (FIG. 56B).

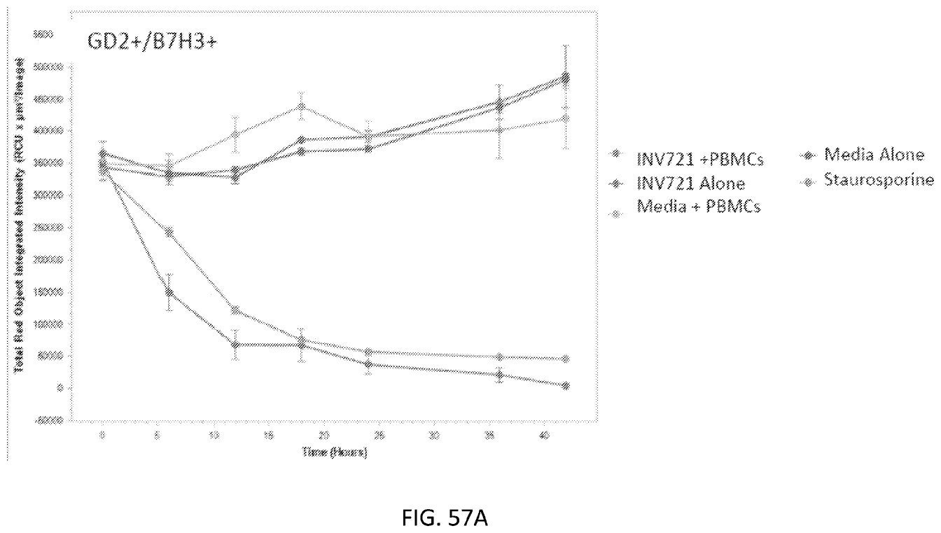

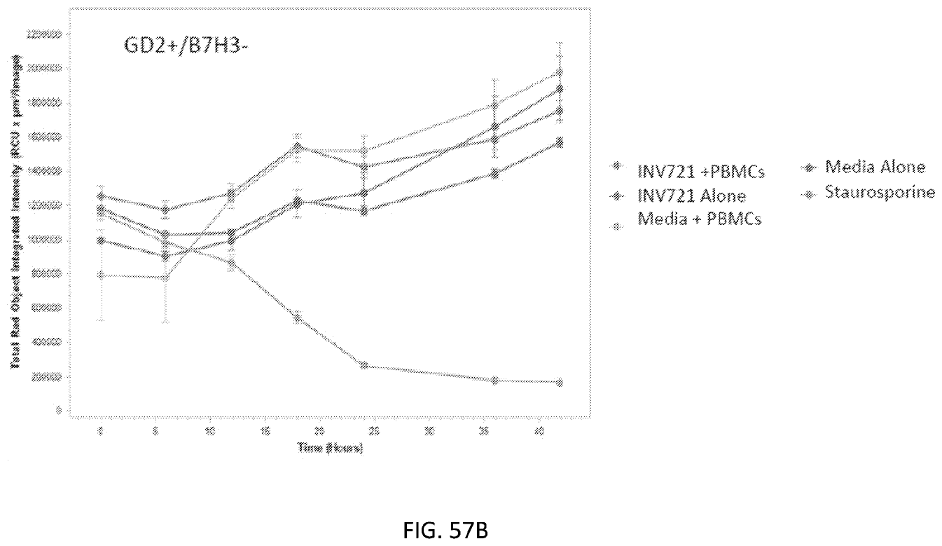

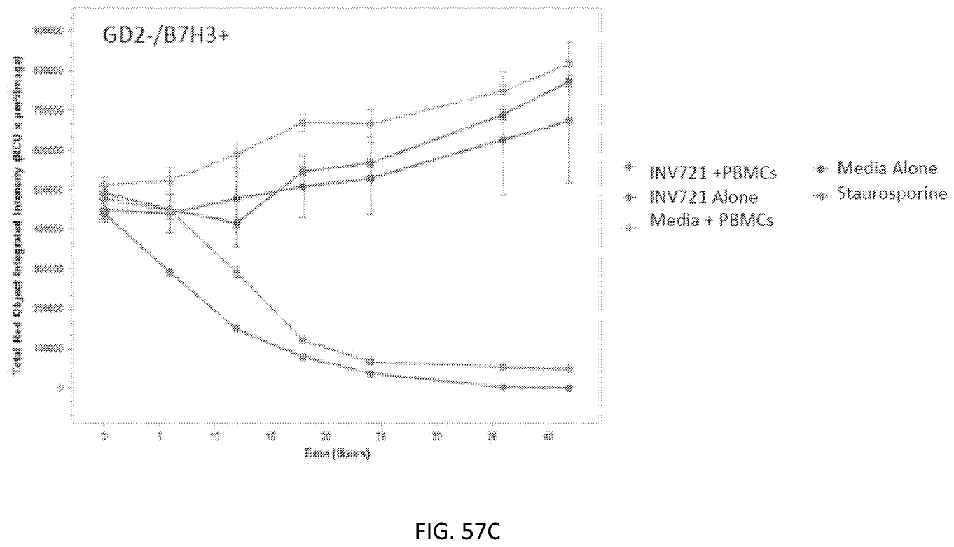

Results of direct antibody-mediated apoptosis are shown in FIGS. 57A-57C. Cells that express B7H3 are killed over time following incubation with INV721 and PBMCs (FIG. 57A and FIG. 57C). Cells that highly express GD2 but do not express B7H3 are not killed following incubation with INV721 and PBMCs (FIG. 57B).

### 6.5.5. Example 5—In Vitro Assessment of INV721 Compared to Anti-B7H3 and Anti-GD2 Monoclonal Antibodies

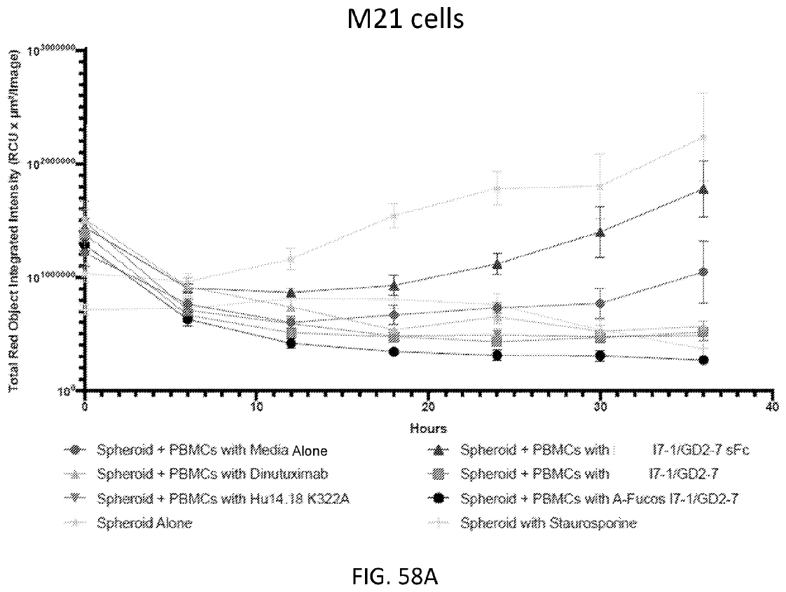

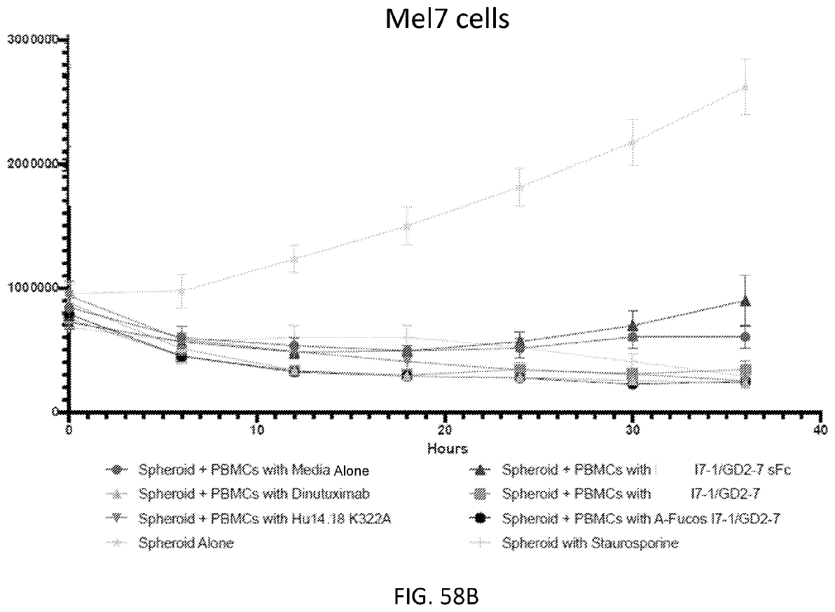

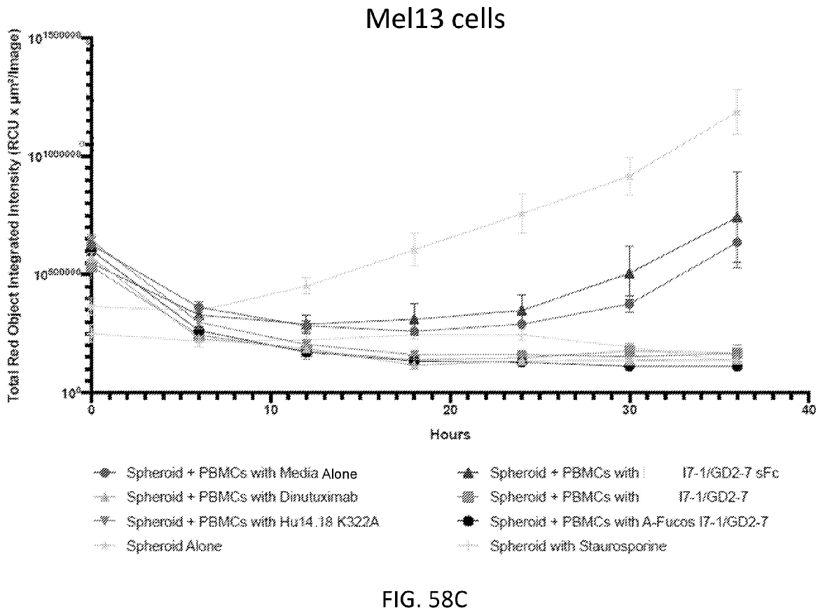

ADCC assays were performed as described in Example 4 to evaluate efficacy of INV721 and the anti-GD2 monoclonal antibodies dinutuximab and hu14.18 in various melanoma cancer cell lines that express both B7H3 and GD2. Results shown in FIG. 58A demonstrate that INV721 has greater efficacy than dinutuximab or Hu14.18 in M21 cells. Results shown in FIGS. 58B-C demonstrate that INV721 is at least as effective as dinutuximab and Hu14.18 in Mel7 (FIG. 58B) and Mel13 (FIG. 58C) cells.

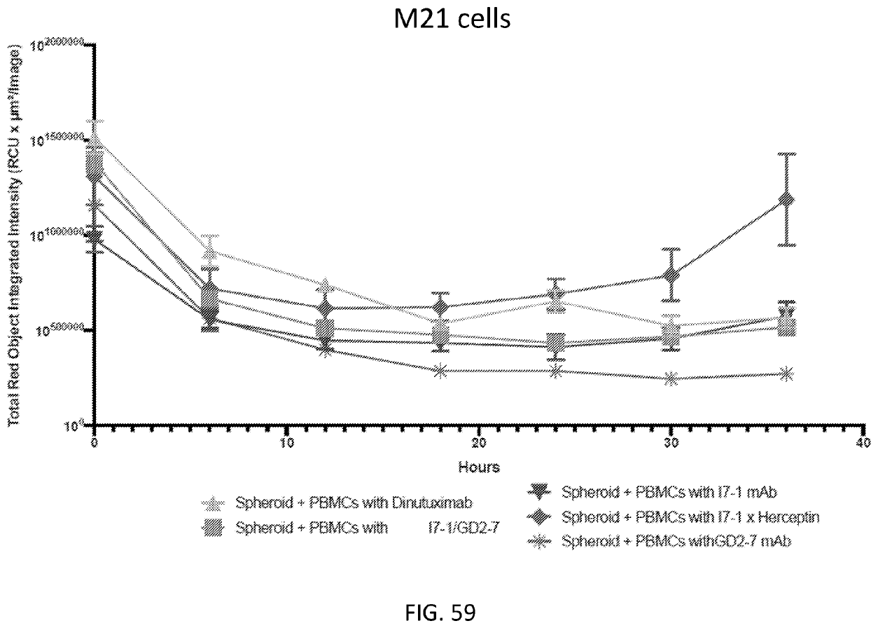

Further ADCC assays were performed to evaluate efficacy of INV721, the anti-GD2 monoclonal antibodies dinutuximab and GD2-7, and the anti-B7H3 antibody I7-01 in M21 cells that express both B7H3 and GD2. Results shown in FIG. 59 demonstrate that INV721 and GD2-7 are more effective at killing than dinutuximab. I7-01 is nearly as effective as INV721.

### 6.5.6. Example 6—In Vivo Assessment of INV721 Binding

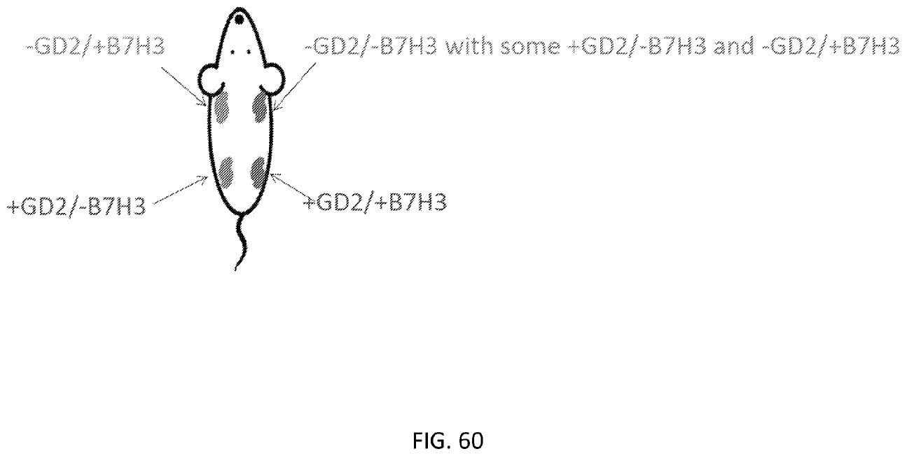

In order to evaluate binding of INV721 in vivo, mice bearing four B78 tumors with differential B7H3 and GD2 expression were treated with zirconium radiolabeled (⁸⁹Zr) dinutuximab, INV721, or a non-specific control antibody (BB-WT) and assessed by positron emission tomography (PET) imaging. As shown in FIG. 60 and with reference to a mouse in the prone position viewed from above with the nose at the top and tail at the bottom: tumors expressing both GD2 and B7H3 were at the bottom right, tumors expressing GD2 but not expressing B7H3 were at the bottom left, tumors expressing B7H3 but not GD2 were at the top left, and tumors that were mostly negative for GD2 and B7H3 expression but had some expression of both antigens were at the top right.

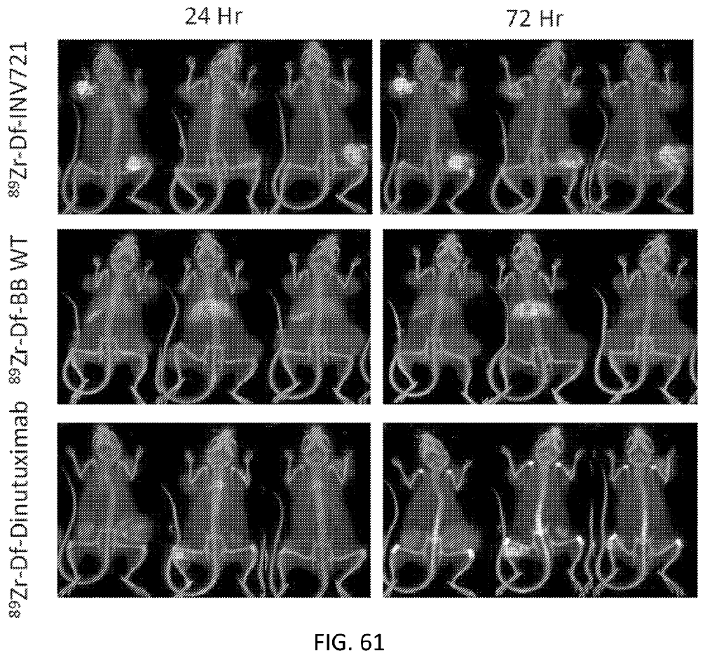

Results in FIG. 61 show that uptake of INV721 was highest in GD2/B7H3 double positive tumors (top panel, bottom right of each mouse) and in tumors that highly express B7H3 but are GD2 negative (top panel, top left of each mouse). Minimal uptake of INV721 is observed in tumors expressing high levels of GD2 but no B7H3 (top panel, bottom left of each mouse). In contrast, high uptake of dinutuximab is seen in tumors that express GD2 but no B7H3 (bottom panel, bottom left of each mouse).

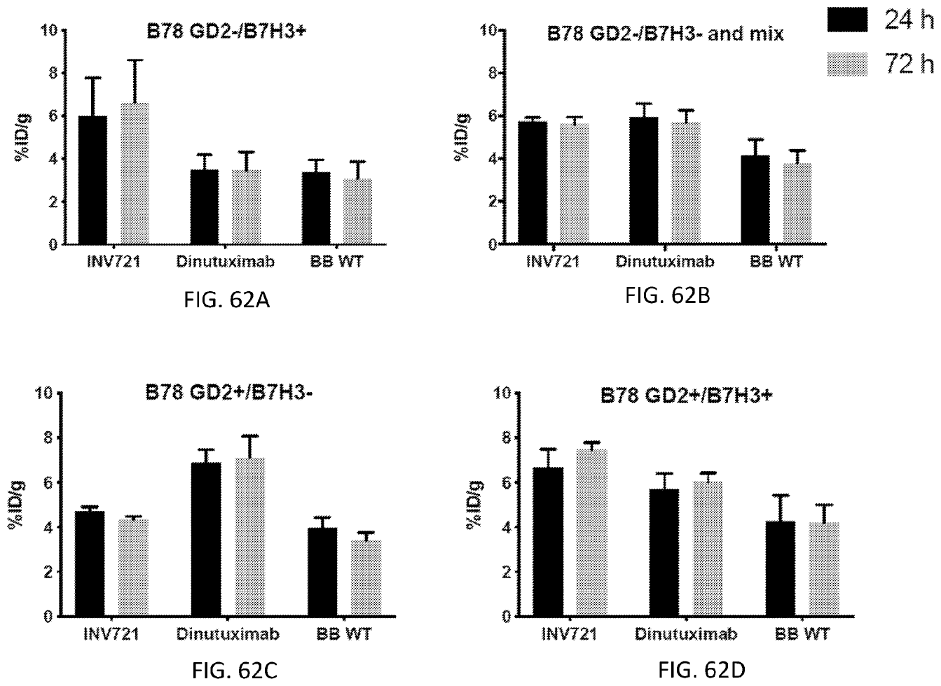

Graphs in FIGS. 62A-62D show the ratio of activity of ⁸⁹Zr-labeled antibodies injected per dose per gram of tumor. INV721 shows increased uptake compared to dinutuximab in tumors expressing both GD2 and B7H3 (FIG. 62D). Uptake of INV721 is also seen in tumors that express B7H3 but do not express GD2 (FIG. 62A). Uptake of dinutuximab but not INV721 is seen in tumors that express GD2 but do not express B7H3 (FIG. 62C).

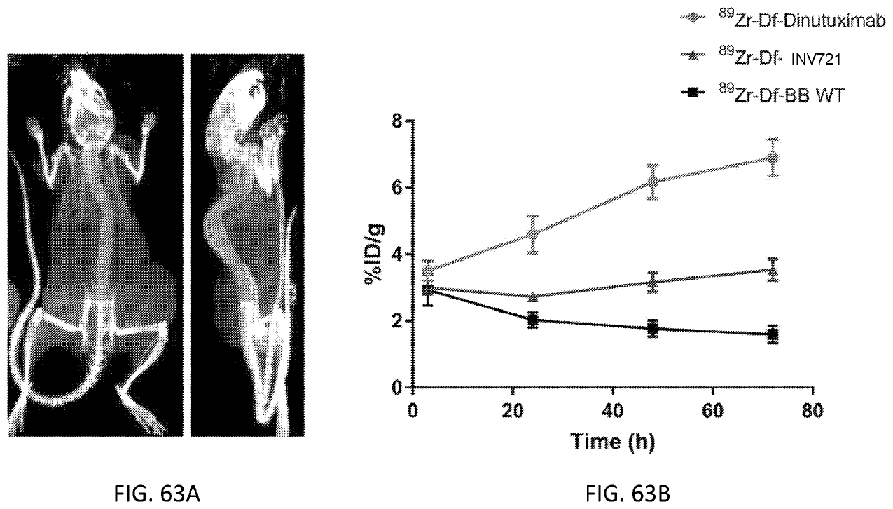

In order to evaluate the amount of antibody that binds to the spine and thus might contribute to pain toxicity, the ratio of activity of ⁸⁹Zr-labeled antibodies injected per dose per gram of tumor was measured for the spine region alone (FIG. 63A) over time for treated animals. Results in FIG. 63B show that significantly less INV721 binds to the spine of treated animals compared to the anti-GD2 monoclonal antibody dinutuximab.

### 6.5.7. Example 7—In Vivo Assessment of INV721 Efficacy

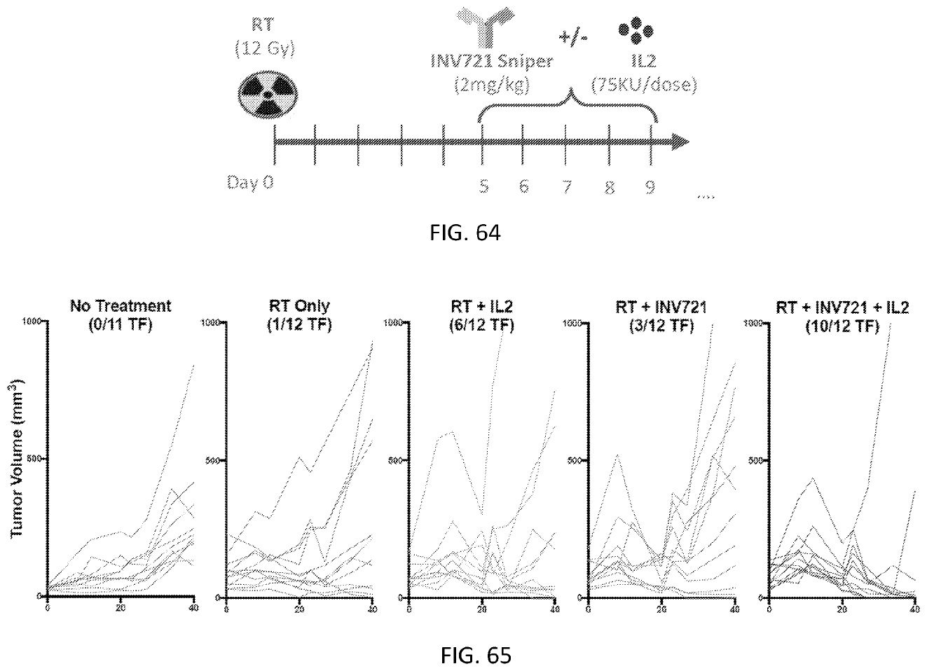

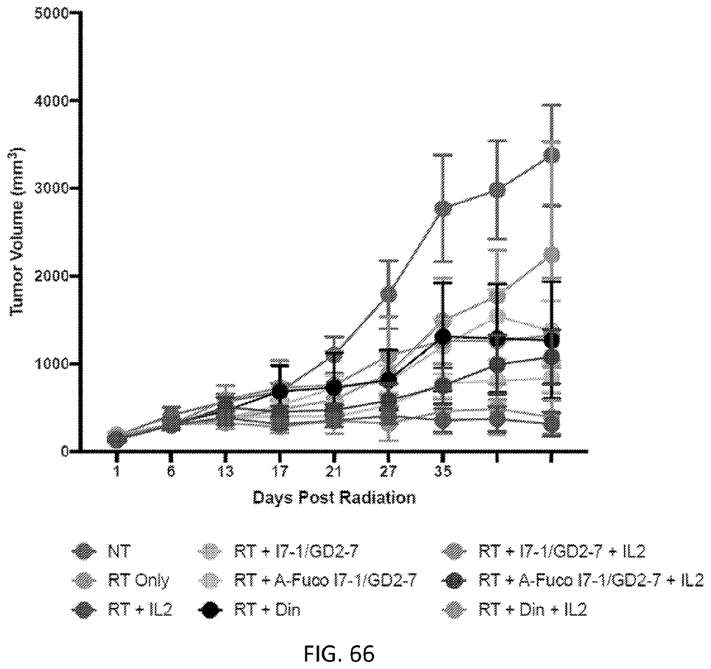

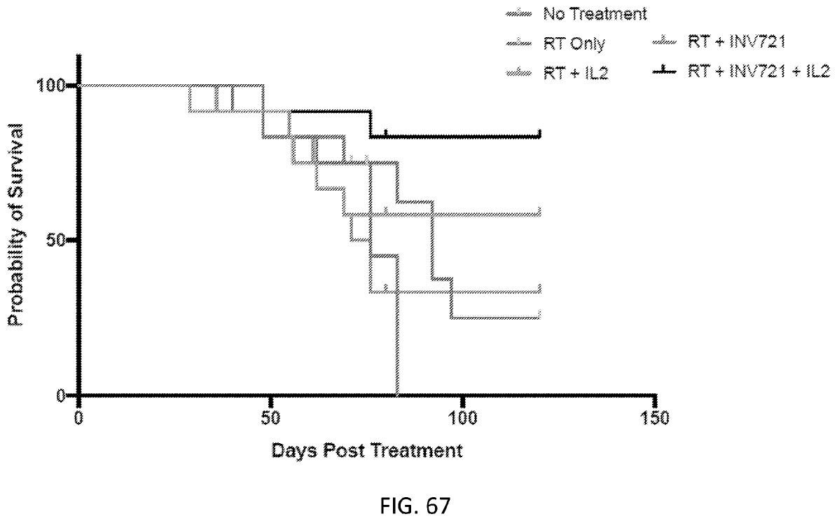

In order to evaluate efficacy of INV721 in vivo, mice bearing B78 melanoma tumors that express GD2 and B7H3 were treated with radiation therapy followed by INV721 and/or IL-2 and were monitored for tumor growth and survival. Briefly, mice bearing 75 mm³ B78 tumors that express B7H3 and GD2 were randomized to receive radiation therapy (12 Gy) at Day 0 followed by INV721 (40 μg/dose) and/or IL-2 (75,000 U/dose) on Days 5-9 (FIG. 64). The results showed that mice treated with radiation therapy, INV721, and IL-2 had improved response as compared to the other treatment groups, with slowed tumor growth and improved tumor free survival (FIG. 65 and FIG. 66) as well as improved overall survival (FIG. 67). Results in FIG. 66 demonstrate improved efficacy of a-fucosylated INV721 compared to fucosylated INV721 (used in all previous studies).

### 7. SEQUENCES

TABLE 1
V region sequences

| SEQ ID | | |
|---|---|---|
| NO | DESCRIPTION | SEQUENCE |
| 1 | 17-01 (anti- | DIQMTQSPSSLSASVGDRVTITCRASQSVSSAVAWYQQKPGK |
| | B7H3) V_{L} | APKLLIYSASSLYSGVPSRFSGSRSGTDFTLTISSLQPEDFATY |
| | domain | YCQQSGRSLYTFGQGTKVEIK |
| 2 | 17-01 (anti- | EVQLVESGGGLVQPGGSLRLSCAASGFTFSTYYIHWVRQAPG |
| | B7H3) V_{H} | KGLEWVAWIHPSGKYTYYADSVKGRFTISADTSKNTAYLQMN |
| | domain | SLRAEDTAVYYCARHYQVGAMDYWGQGTLVTVSS |
| 3 | GD2-05 and | DIQMTQSPSSLSASVGDRVTITCRSSQSLVHRNGNTYLHWYQ |
| | GD2-07 (anti- | QKPGKAPKLLIHKVSNRFSGVPSRFSGSGSGTDFTLTISSLQ |
| | GD2) V_{L} domain | PEDFATYYCSQSTHVPPLTFGQGTKVEIK |
| 4 | GD2-05 and | EVQLVESGGGLVQPGGSLRLSCAASGSSFTGYNMNWVRQAPG |
| | GD2-07 (anti- | KGLEWVAAIDPYYGGTYYADSVKGRFTISADTSKNTAYLQMN |
| | GD2) V_{H} domain | SLRAEDTAVYYCASGMEYWGQGTLVTVSS |

TABLE 2
17-01 VL domain CDRs

| Region | Definition | Sequence Fragment | Residues | SEQ ID NO: |
|---|---|---|---|---|
| CDR-L1 | Chothia | RASQSVSSAVA-- | 24-34 | 5 |
| | AbM | RASQSVSSAVA-- | 24-34 | 6 |
| | Kabat | RASQSVSSAVA-- | 24-34 | 7 |
| | Contact | ------SSAVAWY | 30-36 | 8 |
| | IMGT | ---QSVSSA---- | 27-32 | 9 |
| CDR-L2 | Chothia | ----SASSLYS | 50-56 | 10 |
| | AbM | ----SASSLYS | 50-56 | 11 |
| | Kabat | ----SASSLYS | 50-56 | 12 |
| | Contact | LLIYSASSLY- | 46-55 | 13 |
| | IMGT | ----SA----- | 50-51 | 14 |
| CDR-L3 | Chothia | QQSGRSLYT | 89-97 | 15 |
| | AbM | QQSGRSLYT | 89-97 | 16 |
| | Kabat | QQSGRSLYT | 89-97 | 17 |
| | Contact | QQSGRSLY- | 89-96 | 18 |
| | IMGT | QQSGRSLYT | 89-97 | 19 |

TABLE 3
17-01 VH domain CDRs

| | | | | SEQ ID |
|---|---|---|---|---|
| Region | Definition | Sequence Fragment | Residues | NO: |
| CDR-H1 | Chothia | GFTFSTY--- | 26-32 | 20 |
| | AbM | GFTFSTYYIH | 26-35 | 21 |
| | Kabat | -----TYYIH | 31-35 | 22 |
| | Contact | ----STYYIH | 30-35 | 23 |
| | IMGT | GFTFSTYY-- | 26-33 | 24 |
| CDR-H2 | Chothia | -----HPSGKY--------- | 52-57 | 25 |
| | AbM | ---WIHPSGKYTY------- | 50-59 | 26 |
| | Kabat | ---WIHPSGKYTYYADSVKG | 50-66 | 27 |
| | Contact | WVAWIHPSGKYTY------- | 47-59 | 28 |
| | IMGT | ----IHPSGKYT-------- | 51-58 | 29 |
| CDR-H3 | Chothia | --HYQVGAMDY | 99-107 | 30 |
| | AbM | --HYQVGAMDY | 99-107 | 31 |
| | Kabat | --HYQVGAMDY | 99-107 | 32 |
| | Contact | ARHYQVGAMD- | 97-106 | 33 |
| | IMGT | ARHYQVGAMDY | 97-107 | 34 |
| HFR4 | Chothia | -WGQGTLVTVSS | 108-118 | 35 |
| | AbM | -WGQGTLVTVSS | 108-118 | 36 |
| | Kabat | -WGQGTLVTVSS | 108-118 | 37 |
| | Contact | YWGQGTLVTVSS | 107-118 | 38 |
| | IMGT | -WGQGTLVTVSS | 108-118 | 39 |

TABLE 4
GD2-05 and GD2-07 VL domain CDRs

| Region | Definition | Sequence Fragment | Residues | SEQ ID NO: |
|---|---|---|---|---|
| CDR-L1 | Chothia | RSSQSLVHRNGNTYLH-- | 24-39 | 40 |
| | AbM | RSSQSLVHRNGNTYLH-- | 24-39 | 41 |
| | Kabat | RSSQSLVHRNGNTYLH-- | 24-39 | 42 |
| | Contact | ------VHRNGNTYLHWY | 30-41 | 43 |
| | IMGT | ---QSLVHRNGNTY---- | 27-37 | 44 |
| CDR-L2 | Chothia | ----KVSNRFS | 55-61 | 45 |
| | AbM | ----KVSNRFS | 55-61 | 46 |
| | Kabat | ----KVSNRFS | 55-61 | 47 |
| | Contact | LLIHKVSNRF- | 51-60 | 48 |
| | IMGT | ----KV----- | 55-56 | 49 |
| CDR-L3 | Chothia | SQSTHVPPLT | 94-103 | 50 |
| | AbM | SQSTHVPPLT | 94-103 | 51 |
| | Kabat | SQSTHVPPLT | 94-103 | 52 |
| | Contact | SQSTHVPPL- | 94-102 | 53 |
| | IMGT | SQSTHVPPLT | 94-103 | 54 |

TABLE 5
GD2-05 and GD2-07 VH domain CDRs

| Region | Definition | Sequence Fragment | Residues | SEQ ID NO: |
|---|---|---|---|---|
| CDR-H1 | Chothia | GSSFTGY--- | 26-32 | 55 |
| | AbM | GSSFTGYNMN | 26-35 | 56 |
| | Kabat | -----GYNMN | 31-35 | 57 |
| | Contact | ----TGYNMN | 30-35 | 58 |
| | IMGT | GSSFTGYN-- | 26-33 | 59 |
| CDR-H2 | Chothia | -----DPYYGG--------- | 52-57 | 60 |
| | AbM | ---AIDPYYGGTY------- | 50-59 | 61 |
| | Kabat | ---AIDPYYGGTYYADSVKG | 50-66 | 62 |
| | Contact | WVAAIDPYYGGTY------- | 47-59 | 63 |
| | IMGT | ----IDPYYGGT-------- | 51-58 | 64 |
| CDR-H3 | Chothia | --GMEY | 99-102 | 65 |
| | AbM | --GMEY | 99-102 | 66 |
| | Kabat | --GMEY | 99-102 | 67 |
| | Contact | ASGME- | 97-101 | 68 |
| | IMGT | ASGMEY | 97-102 | 69 |

### 8. EQUIVALENTS AND INCORPORATION BY REFERENCE

All references cited herein are incorporated by reference to the same extent as if each individual publication, database entry (e.g. Genbank sequences or GenelD entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

1. A multi-specific antibody construct, comprising:
a first antigen binding site (ABS) specific for a first tumor cell surface antigen, wherein the first tumor cell antigen is B7 Homolog 3 (B7H3); and
a second antigen binding site (ABS) specific for a second tumor cell surface antigen, wherein the second tumor cell antigen is disialoganglioside (GD2).

2. The multi-specific antibody construct of claim 1, wherein the first ABS binds human B7H3 with a K_{D} that is greater than 10 nM, the second ABS binds human GD2 with a K_{D}that is greater than 10 nM, and wherein the antibody construct binds to a tumor cell expressing B7H3 and GD2 with a K_{D} that is less than 100 nM.

3. The multi-specific antibody construct of claim 2, wherein the antibody construct exhibits lower binding to cells that express GD2 but not B7H3 compared to cells that express both GD2 and B7H3.

4. The multi-specific antibody construct of claim 3, wherein the antibody construct has greater antibody-dependent cellular cytotoxicity (ADCC) activity against a cell that expresses B7H3 and GD2 as compared to ADCC activity against a cell that expresses GD2 but does not express B7H3.

5. The multi-specific antibody construct of claim 1, wherein the antibody constructs bind less to nerve cells as compared to dinutuximab at comparable concentrations.

6. The multi-specific antibody construct of claim 1, wherein the first ABS comprises:
a) a first heavy chain variable region (VH) CDR1,
b) a first VH CDR2,
c) a first VH CDR3,
d) a first light chain variable region (VL) CDR1,
e) a first VL CDR2, and
f) a first VL CDR3.

7. The multi-specific antibody construct of claim 6, wherein the first ABS comprises:
a) a first VH CDR1 with the amino acid sequence of SEQ ID NO:22
b) a first VH CDR2 with the amino acid sequence of SEQ ID NO:27,
c) a first VH CDR3 with the amino acid sequence of SEQ ID NO:32,
d) a first VL CDR1 with the amino acid sequence of SEQ ID NO:7,
e) a first VL CDR2 with the amino acid sequence of SEQ ID NO:12, and
f) a first VL CDR3 with the amino acid sequence of SEQ ID NO:17.

8. The multi-specific antibody construct of claim 1, wherein the first ABS comprises:
a first heavy chain variable region (VH) with the amino acid sequence of SEQ ID NO: 2 and a first light chain variable region (VL) with the amino acid sequence of SEQ ID NO: 1.

9. The multi-specific antibody construct of claim 1, wherein the second ABS comprises:
a) a second heavy chain variable region (VH) CDR1,
b) a second VH CDR2,
c) a second VH CDR3,
d) a second light chain variable region (VL) CDR1,
e) a second VL CDR2, and
f) a second VL CDR3.

10. The multi-specific antibody construct of claim 9, wherein the second ABS comprises:
a) a second VH CDR1 with the amino acid sequence of SEQ ID NO:57,
b) a second VH CDR2 with the amino acid sequence of SEQ ID NO:62,
c) a second VH CDR3 with the amino acid sequence of SEQ ID NO:67,
d) a second VL CDR1 with the amino acid sequence of SEQ ID NO:42,
e) a second VL CDR2 with the amino acid sequence of SEQ ID NO:47, and
f) a second VL CDR3 with the amino acid sequence of SEQ ID NO:52.

11. The multi-specific antibody construct of claim 1, wherein the second ABS comprises:
a second heavy chain variable region (VH) with the amino acid sequence of SEQ ID NO: 4 and a second light chain variable region (VL) with the amino acid sequence of SEQ ID NO: 3.

12. The antibody construct of any of the preceding claims claim 1, wherein the antibody construct comprises a first, second, third, and fourth polypeptide chain, wherein:
a) the first polypeptide chain comprises a domain A, a domain B, a domain D, and a domain E, wherein the domains are arranged, from N-terminus to C-terminus, in a A-B-D-E orientation, wherein domain A comprises a variable region domain amino acid sequence, and wherein domain B, domain D, and domain E each comprise a constant region domain amino acid sequence;
b) the second polypeptide chain comprises a domain F and a domain G, wherein the domains are arranged, from N-terminus to C-terminus, in a F-G orientation, and wherein domain F has a variable region domain amino acid sequence and domain G comprises a constant region domain amino acid sequence;
c) the third polypeptide chain comprises a domain H, a domain I, a domain J, and a domain K, wherein the domains are arranged, from N-terminus to C-terminus, in a H-I-J-K orientation, wherein domain H has a variable region domain amino acid sequence, and wherein domain I, domain J, and domain K each have a constant region amino acid sequence;
d) the fourth polypeptide chain comprises a domain L and a domain M, wherein the domains are arranged, from N-terminus to C-terminus, in a L-M orientation, and wherein domain L has a variable region domain amino acid sequence and domain M comprises a constant region amino acid sequence, or portion thereof;
e) the first and second polypeptides are associated through an interaction between the A and F domains and an interaction between the B and G domains;
f) the third and fourth polypeptides are associated through an interaction between the H and L domains and an interaction between the I and M domains; and
g) the first and third polypeptides are associated through an interaction between the D and J domains and an interaction between the E and K domains.

13. The antibody construct of claim 12, wherein:
domain A is a V_{L} domain;
domain B comprises a CH3 domain;
domain D is a CH2 domain;
domain E is a CH3 domain;
domain F is a V_{H} domain;
domain G comprises a CH3 domain;
domain H is a V_{L} domain;
domain I is a C_{L} domain;
domain J is a CH2 domain;
domain K is a CH3 domain;
domain L is a V_{H} domain; and
domain M is a CH1 domain.

14. The antibody construct of claim 13, wherein:
domains D and J have the amino acid sequence of human IgG1 CH2 domain;
domain I has the amino acid sequence of human C kappa light chain; and
domain M has the amino acid sequence of human IgG1 CH1 region.

15. The antibody construct of claim 14, wherein:
domain B has a CH3 amino acid sequence with a T366K mutation and a C-terminal extension incorporating a KSC tripeptide sequence followed by the DKTHT motif (SEQ ID NO: 74) of an IgG1 hinge region;
domain E has a CH3 amino acid sequence with a S354C and a T366W mutation;
domain G has a CH3 amino acid sequence with a L351D mutation and a C-terminal extension incorporating a GEC amino acid disulfide motif; and
domain K has a CH3 amino acid sequence with a Y349C, a T366S, a L368A, a Y407V mutation, and optionally a D356E and a L358M mutation.

16. The antibody construct of claim 14, wherein:
domain B has a CH3 amino acid sequence and a C-terminal extension incorporating a KSC tripeptide sequence followed by the DKTHT motif (SEQ ID NO: 74) of an IgG1 hinge region;
domain E has a CH3 amino acid sequence with a S354C and a T366W mutation;
domain G has a CH3 amino acid sequence and a C-terminal extension incorporating a GEC amino acid disulfide motif; and
domain K has a CH3 amino acid sequence with a Y349C, a T366S, a L368A, a Y407V mutation, and optionally a D356E and a L358M mutation.

17. The antibody construct of claim 14, wherein:
domain B has a CH3 amino acid sequence with a Y349C mutation and a C-terminal extension incorporating a PGK tripeptide sequence that is followed by the DKTHT motif (SEQ ID NO: 74) of an IgG1 hinge region;
domain E has a CH3 amino acid sequence with a S354C and a T366W mutation;
domain G has a CH3 amino acid sequence with a S354C mutation and a C-terminal extension incorporating a PGK tripeptide sequence; and
domain K has a CH3 amino acid sequence with a Y349C, a T366S, a L368A, a Y407V mutation, and optionally a D356E and a L358M mutation.

18. The antibody construct of claim 14, wherein:
domain B has a CH3 amino acid sequence with a P343V mutation, a Y349C mutation, and a C-terminal extension incorporating a PGK tripeptide sequence that is followed by the DKTHT motif (SEQ ID NO: 74) of an IgG1 hinge region;
domain E has a CH3 amino acid sequence with a S354C and a T366W mutation;
domain G has a CH3 amino acid sequence with a S354C mutation and a C-terminal extension incorporating a PGK tripeptide sequence; and
domain K has a CH3 amino acid sequence with a Y349C, a T366S, a L368A, a Y407V mutation, and optionally a D356E and a L358M mutation.

19. The antibody construct of claim 15, wherein the first ABS is formed by domains A and F and the second ABS is formed by domains H and L.

20. The antibody construct of claim 1, wherein the antibody construct is conjugated to a therapeutic agent.

21. A pharmaceutical composition comprising an effective amount of the multi-specific antibody construct of claim 1 and a pharmaceutically acceptable carrier.

22. A method of treating a proliferative disease in a human subject, comprising administering to the human subject a pharmaceutical composition of claim 21.

23. The method of claim 22, wherein the proliferative disease is cancer.

24. The method of claim 23, wherein the cancer is neuroblastoma, glioblastoma, small cell lung cancer, or sarcoma.

25. The method of claim 22, wherein the administering results in decreased pain compared to treatment with an anti-GD2 monoclonal antibody.

26. The method of claim 25, wherein the anti-GD2 monoclonal antibody is dinutuximab or hu14.18.

27. A method of selectively targeting a tumor cell in a subject, comprising administering to the subject a pharmaceutical composition of claim 21.


SEQUENCE LISTING (78 sequences)

SEQ ID NO: 1 - protein, 107 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSGRSLYTFGQGTKVEIK

SEQ ID NO: 2 - protein, 118 aa, Artificial Sequence
  Description of Artificial Sequence Synthetic polypeptide
EVQLVESGGGLVQPGGSLRLSCAASGFTFSTYYIHWVRQAPGKGLEWVAWIHPSGKYTYYADSVKGRFTISADTSKNTAYLQMNSLRAEDTAVYYCARHYQVGAMDYWGQGTLVTVSS

SEQ ID NO: 3 - protein, 113 aa, Artificial Sequence
  Description of Artificial Sequence Synthetic polypeptide
DIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIK

SEQ ID NO: 4 - protein, 113 aa, Artificial Sequence
  Description of Artificial Sequence Synthetic polypeptide
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCASGMEYWGQGTLVTVSS

SEQ ID NO: 5 - protein, 11 aa, Artificial Sequence
  Description of Artificial Sequence Synthetic peptide
RASQSVSSAVA

SEQ ID NO: 6 - protein, 11 aa, Artificial Sequence
  Description of Artificial Sequence Synthetic peptide
RASQSVSSAVA

SEQ ID NO: 7 - protein, 11 aa, Artificial Sequence
  Description of Artificial Sequence Synthetic peptide
RASQSVSSAVA

SEQ ID NO: 8 - protein, 7 aa, Artificial Sequence
  Description of Artificial Sequence Synthetic peptide
SSAVAWY

SEQ ID NO: 9 - protein, 6 aa, Artificial Sequence
  Description of Artificial Sequence Synthetic peptide
QSVSSA

SEQ ID NO: 10 - protein, 7 aa, Artificial Sequence
  Description of Artificial Sequence Synthetic peptide
SASSLYS

SEQ ID NO: 11 - protein, 7 aa, Artificial Sequence
  Description of Artificial Sequence Synthetic peptide
SASSLYS

SEQ ID NO: 12 - protein, 7 aa, Artificial Sequence
  Description of Artificial Sequence Synthetic peptide
SASSLYS

SEQ ID NO: 13 - protein, 10 aa, Artificial Sequence
  Description of Artificial Sequence Synthetic peptide
LLIYSASSLY

SEQ ID NO: 14 - protein, 2 aa, Artificial Sequence
  Description of Artificial Sequence Synthetic peptide
SA

SEQ ID NO: 15 - protein, 9 aa, Artificial Sequence
  Description of Artificial Sequence Synthetic peptide
QQSGRSLYT

SEQ ID NO: 16 - protein, 9 aa, Artificial Sequence
  Description of Artificial Sequence Synthetic peptide
QQSGRSLYT

SEQ ID NO: 17 - protein, 9 aa, Artificial Sequence
  Description of Artificial Sequence Synthetic peptide
QQSGRSLYT

SEQ ID NO: 18 - protein, 8 aa, Artificial Sequence
  Description of Artificial Sequence Synthetic peptide
QQSGRSLY

SEQ ID NO: 19 - protein, 9 aa, Artificial Sequence
  Description of Artificial Sequence Synthetic peptide
QQSGRSLYT

SEQ ID NO: 20 - protein, 7 aa, Artificial Sequence
  Description of Artificial Sequence Synthetic peptide
GFTFSTY

SEQ ID NO: 21 - protein, 10 aa, Artificial Sequence
  Description of Artificial Sequence Synthetic peptide
GFTFSTYYIH

SEQ ID NO: 22 - protein, 5 aa, Artificial Sequence
  Description of Artificial Sequence Synthetic peptide
TYYIH

SEQ ID NO: 23 - protein, 6 aa, Artificial Sequence
  Description of Artificial Sequence Synthetic peptide
STYYIH

SEQ ID NO: 24 - protein, 8 aa, Artificial Sequence
  Description of Artificial Sequence Synthetic peptide
GFTFSTYY

SEQ ID NO: 25 - protein, 6 aa, Artificial Sequence
  Description of Artificial Sequence Synthetic peptide
HPSGKY

SEQ ID NO: 26 - protein, 10 aa, Artificial Sequence
  Description of Artificial Sequence Synthetic peptide
WIHPSGKYTY

SEQ ID NO: 27 - protein, 17 aa, Artificial Sequence
  Description of Artificial Sequence Synthetic peptide
WIHPSGKYTYYADSVKG

SEQ ID NO: 28 - protein, 13 aa, Artificial Sequence
  Description of Artificial Sequence Synthetic peptide
WVAWIHPSGKYTY

SEQ ID NO: 29 - protein, 8 aa, Artificial Sequence
  Description of Artificial Sequence Synthetic peptide
IHPSGKYT

SEQ ID NO: 30 - protein, 9 aa, Artificial Sequence
  Description of Artificial Sequence Synthetic peptide
HYQVGAMDY

SEQ ID NO: 31 - protein, 9 aa, Artificial Sequence
  Description of Artificial Sequence Synthetic peptide
HYQVGAMDY

SEQ ID NO: 32 - protein, 9 aa, Artificial Sequence
  Description of Artificial Sequence Synthetic peptide
HYQVGAMDY

SEQ ID NO: 33 - protein, 10 aa, Artificial Sequence
  Description of Artificial Sequence Synthetic peptide
ARHYQVGAMD

SEQ ID NO: 34 - protein, 11 aa, Artificial Sequence
  Description of Artificial Sequence Synthetic peptide
ARHYQVGAMDY

SEQ ID NO: 35 - protein, 11 aa, Artificial Sequence
  Description of Artificial Sequence Synthetic peptide
WGQGTLVTVSS

SEQ ID NO: 36 - protein, 11 aa, Artificial Sequence
  Description of Artificial Sequence Synthetic peptide
WGQGTLVTVSS

SEQ ID NO: 37 - protein, 11 aa, Artificial Sequence
  Description of Artificial Sequence Synthetic peptide
WGQGTLVTVSS

SEQ ID NO: 38 - protein, 12 aa, Artificial Sequence
  Description of Artificial Sequence Synthetic peptide
YWGQGTLVTVSS

SEQ ID NO: 39 - protein, 11 aa, Artificial Sequence
  Description of Artificial Sequence Synthetic peptide
WGQGTLVTVSS

SEQ ID NO: 40 - protein, 16 aa, Artificial Sequence
  Description of Artificial Sequence Synthetic peptide
RSSQSLVHRNGNTYLH

SEQ ID NO: 41 - protein, 16 aa, Artificial Sequence
  Description of Artificial Sequence Synthetic peptide
RSSQSLVHRNGNTYLH

SEQ ID NO: 42 - protein, 16 aa, Artificial Sequence
  Description of Artificial Sequence Synthetic peptide
RSSQSLVHRNGNTYLH

SEQ ID NO: 43 - protein, 12 aa, Artificial Sequence
  Description of Artificial Sequence Synthetic peptide
VHRNGNTYLHWY

SEQ ID NO: 44 - protein, 11 aa, Artificial Sequence
  Description of Artificial Sequence Synthetic peptide
QSLVHRNGNTY

SEQ ID NO: 45 - protein, 7 aa, Artificial Sequence
  Description of Artificial Sequence Synthetic peptide
KVSNRFS

SEQ ID NO: 46 - protein, 7 aa, Artificial Sequence
  Description of Artificial Sequence Synthetic peptide
KVSNRFS

SEQ ID NO: 47 - protein, 7 aa, Artificial Sequence
  Description of Artificial Sequence Synthetic peptide
KVSNRFS

SEQ ID NO: 48 - protein, 10 aa, Artificial Sequence
  Description of Artificial Sequence Synthetic peptide
LLIHKVSNRF

SEQ ID NO: 49 - protein, 2 aa, Artificial Sequence
  Description of Artificial Sequence Synthetic peptide
KV

SEQ ID NO: 50 - protein, 10 aa, Artificial Sequence
  Description of Artificial Sequence Synthetic peptide
SQSTHVPPLT

SEQ ID NO: 51 - protein, 10 aa, Artificial Sequence
  Description of Artificial Sequence Synthetic peptide
SQSTHVPPLT

SEQ ID NO: 52 - protein, 10 aa, Artificial Sequence
  Description of Artificial Sequence Synthetic peptide
SQSTHVPPLT

SEQ ID NO: 53 - protein, 9 aa, Artificial Sequence
  Description of Artificial Sequence Synthetic peptide
SQSTHVPPL

SEQ ID NO: 54 - protein, 10 aa, Artificial Sequence
  Description of Artificial Sequence Synthetic peptide
SQSTHVPPLT

SEQ ID NO: 55 - protein, 7 aa, Artificial Sequence
  Description of Artificial Sequence Synthetic peptide
GSSFTGY

SEQ ID NO: 56 - protein, 10 aa, Artificial Sequence
  Description of Artificial Sequence Synthetic peptide
GSSFTGYNMN

SEQ ID NO: 57 - protein, 5 aa, Artificial Sequence
  Description of Artificial Sequence Synthetic peptide
GYNMN

SEQ ID NO: 58 - protein, 6 aa, Artificial Sequence
  Description of Artificial Sequence Synthetic peptide
TGYNMN

SEQ ID NO: 59 - protein, 8 aa, Artificial Sequence
  Description of Artificial Sequence Synthetic peptide
GSSFTGYN

SEQ ID NO: 60 - protein, 6 aa, Artificial Sequence
  Description of Artificial Sequence Synthetic peptide
DPYYGG

SEQ ID NO: 61 - protein, 10 aa, Artificial Sequence
  Description of Artificial Sequence Synthetic peptide
AIDPYYGGTY

SEQ ID NO: 62 - protein, 17 aa, Artificial Sequence
  Description of Artificial Sequence Synthetic peptide
AIDPYYGGTYYADSVKG

SEQ ID NO: 63 - protein, 13 aa, Artificial Sequence
  Description of Artificial Sequence Synthetic peptide
WVAAIDPYYGGTY

SEQ ID NO: 64 - protein, 8 aa, Artificial Sequence
  Description of Artificial Sequence Synthetic peptide
IDPYYGGT

SEQ ID NO: 65 - protein, 4 aa, Artificial Sequence
  Description of Artificial Sequence Synthetic peptide
GMEY

SEQ ID NO: 66 - protein, 4 aa, Artificial Sequence
  Description of Artificial Sequence Synthetic peptide
GMEY

SEQ ID NO: 67 - protein, 4 aa, Artificial Sequence
  Description of Artificial Sequence Synthetic peptide
GMEY

SEQ ID NO: 68 - protein, 5 aa, Artificial Sequence
  Description of Artificial Sequence Synthetic peptide
ASGME

SEQ ID NO: 69 - protein, 6 aa, Artificial Sequence
  Description of Artificial Sequence Synthetic peptide
ASGMEY

SEQ ID NO: 70 - protein, 443 aa, Artificial Sequence
  Description of Artificial Sequence Synthetic polypeptide
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCARGMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 663 aa, Artificial Sequence
  Description of Artificial Sequence Synthetic polypeptide
EVQLVESGGGLVQPGGSLRLSCAASGFSFTGYNMNWVRQAPGKGLEWVAAIDPYYGGTYYADSVKGRFTISADTSKNTAYLQMNSLRAEDTAVYYCARGMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIQMTQSPSSLSASVGDRVTITCRSSQSLVHRNGNTYLHWYQQKPGKAPKLLIHKVSNRFSGVPSRFSGSGSGTDFTLTISSLQPEDFATYYCSQSTHVPPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 583 aa, Artificial Sequence
  Description of Artificial Sequence Synthetic polypeptide
EVQLVESGGGLVQPGGSLRLSCAASGSSFTGYNMNWVRQAPGKGLEWVAAIDPYYGGTYYADSVKGRFTISADTSKNTAYLQMNSLRAEDTAVYYCASGMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSSLQPEDFATYYCSQSTHVPPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 583 aa, Artificial Sequence
  Description of Artificial Sequence Synthetic polypeptide
EVQLVESGGGLVQPGGSLRLSCAASGFSFTGYNMNWVRQAPGKGLEWVAAIDPYYGGTYYADSVKGRFTISADTSKNTAYLQMNSLRAEDTAVYYCASGMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSSLQPEDFATYYCSQSTHVPPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 5 aa, Artificial Sequence
  Description of Artificial Sequence Synthetic peptide
DKTHT

SEQ ID NO: 75 - protein, 4 aa, Artificial Sequence
  Description of Artificial Sequence Synthetic peptide
AGKC

SEQ ID NO: 76 - protein, 4 aa, Artificial Sequence
  Description of Artificial Sequence Synthetic peptide
PGKC

SEQ ID NO: 77 - protein, 5 aa, Artificial Sequence
  Description of Artificial Sequence Synthetic peptide
AGKGC

SEQ ID NO: 78 - protein, 6 aa, Artificial Sequence
  Description of Artificial Sequence Synthetic peptide
AGKGSC